SEQUENCE LISTING (711 sequences)

SEQ ID NO: 1 - DNA, 27 bp, Glycine max
  source 1..27: mol_type = genomic DNA
tttagacttatacagcgattagctcat

SEQ ID NO: 2 - DNA, 27 bp, Glycine max
  source 1..27: mol_type = genomic DNA
tttagaactttaatttacactactggc

SEQ ID NO: 3 - DNA, 27 bp, Glycine max
  source 1..27: mol_type = genomic DNA
tttaatagaagggtcaattggaggcac

SEQ ID NO: 4 - DNA, 27 bp, Glycine max
  source 1..27: mol_type = genomic DNA
tttagattgagaaagagacttcaagca

SEQ ID NO: 5 - DNA, 27 bp, Glycine max
  source 1..27: mol_type = genomic DNA
tttgcatgtataacactatccaaatcc

SEQ ID NO: 6 - DNA, 27 bp, Glycine max
  source 1..27: mol_type = genomic DNA
tttggtgatataatttatcccaaacca

SEQ ID NO: 7 - DNA, 27 bp, Glycine max
  source 1..27: mol_type = genomic DNA
tttggaagctaaacttctattagacac

SEQ ID NO: 8 - DNA, 27 bp, Glycine max
  source 1..27: mol_type = genomic DNA
tttccaaacacggctcatatataacgc

SEQ ID NO: 9 - DNA, 27 bp, Glycine max
  source 1..27: mol_type = genomic DNA
tttggtaaccaccatggggaactttgt

SEQ ID NO: 10 - DNA, 27 bp, Glycine max
  source 1..27: mol_type = genomic DNA
tttagaggaattaattgatacctgaaa

SEQ ID NO: 11 - DNA, 27 bp, Glycine max
  source 1..27: mol_type = genomic DNA
tttggatccagtctatgatataatgcg

SEQ ID NO: 12 - DNA, 27 bp, Glycine max
  source 1..27: mol_type = genomic DNA
tttgggagatcagctggcggaggaggc

SEQ ID NO: 13 - DNA, 27 bp, Glycine max
  source 1..27: mol_type = genomic DNA
tttagatgaataaacctagtctgtttc

SEQ ID NO: 14 - DNA, 27 bp, Glycine max
  source 1..27: mol_type = genomic DNA
tttggtggagagggtgtgatgtgaatc

SEQ ID NO: 15 - DNA, 27 bp, Glycine max
  source 1..27: mol_type = genomic DNA
tttggttacgaagggtgcaatcacacc

SEQ ID NO: 16 - DNA, 27 bp, Glycine max
  source 1..27: mol_type = genomic DNA
tttagacggtaacccacgtgaaggtca

SEQ ID NO: 17 - DNA, 27 bp, Glycine max
  source 1..27: mol_type = genomic DNA
tttaggctgagggaccaatatatatgt

SEQ ID NO: 18 - DNA, 27 bp, Glycine max
  source 1..27: mol_type = genomic DNA
tttagggctttagccaaacccgtctag

SEQ ID NO: 19 - DNA, 27 bp, Glycine max
  source 1..27: mol_type = genomic DNA
tttagcgcatgaagatgtctgcaaata

SEQ ID NO: 20 - DNA, 27 bp, Glycine max
  source 1..27: mol_type = genomic DNA
tttcgcgtgagccagatatgctcgtga

SEQ ID NO: 21 - DNA, 27 bp, Glycine max
  source 1..27: mol_type = genomic DNA
tttgggagaaagagttgcagaacacag

SEQ ID NO: 22 - DNA, 27 bp, Glycine max
  source 1..27: mol_type = genomic DNA
tttaggtgaacagaacagaagctgaaa

SEQ ID NO: 23 - DNA, 27 bp, Glycine max
  source 1..27: mol_type = genomic DNA
tttggtgtctcctcctatataagtagc

SEQ ID NO: 24 - DNA, 27 bp, Glycine max
  source 1..27: mol_type = genomic DNA
tttggttgtacagataacagtgccaat

SEQ ID NO: 25 - DNA, 27 bp, Glycine max
  source 1..27: mol_type = genomic DNA
tttggaaatacatcatgcacctgtaaa

SEQ ID NO: 26 - DNA, 27 bp, Glycine max
  source 1..27: mol_type = genomic DNA
tttggagcataaggtagctcaagtctt

SEQ ID NO: 27 - DNA, 27 bp, Glycine max
  source 1..27: mol_type = genomic DNA
tttagagtaatttgaatccctccatat

SEQ ID NO: 28 - DNA, 27 bp, Glycine max
  source 1..27: mol_type = genomic DNA
tttggcgtttgaagtgatgtcacctgg

SEQ ID NO: 29 - DNA, 27 bp, Glycine max
  source 1..27: mol_type = genomic DNA
tttgggagttgtaatgatcgccacata

SEQ ID NO: 30 - DNA, 27 bp, Glycine max
  source 1..27: mol_type = genomic DNA
tttggtacaacaattgtattacactct

SEQ ID NO: 31 - DNA, 27 bp, Glycine max
  source 1..27: mol_type = genomic DNA
tttagtgtatgtttacaagaagatttc

SEQ ID NO: 32 - DNA, 27 bp, Glycine max
  source 1..27: mol_type = genomic DNA
tttagaaagttgagctgatcatcaaag

SEQ ID NO: 33 - DNA, 27 bp, Glycine max
  source 1..27: mol_type = genomic DNA
tttggaatcttgtgactcaggttacat

SEQ ID NO: 34 - DNA, 27 bp, Glycine max
  source 1..27: mol_type = genomic DNA
tttagagagacagaattaagtgagaaa

SEQ ID NO: 35 - DNA, 27 bp, Glycine max
  source 1..27: mol_type = genomic DNA
tttagataatagatcgagtttacgctc

SEQ ID NO: 36 - DNA, 27 bp, Glycine max
  source 1..27: mol_type = genomic DNA
tttagatcaattcgatagttcgaataa

SEQ ID NO: 37 - DNA, 27 bp, Glycine max
  source 1..27: mol_type = genomic DNA
tttaggcattaagattgagctaacaag

SEQ ID NO: 38 - DNA, 27 bp, Glycine max
  source 1..27: mol_type = genomic DNA
tttaggtgaattaattaaagagggctg

SEQ ID NO: 39 - DNA, 27 bp, Glycine max
  source 1..27: mol_type = genomic DNA
tttcgtccacggtgctcttgaaccatt

SEQ ID NO: 40 - DNA, 27 bp, Glycine max
  source 1..27: mol_type = genomic DNA
tttgcacccaacagtagatgcctaggc

SEQ ID NO: 41 - DNA, 27 bp, Glycine max
  source 1..27: mol_type = genomic DNA
tttagagtctactctctccggtcctat

SEQ ID NO: 42 - DNA, 27 bp, Glycine max
  source 1..27: mol_type = genomic DNA
tttggaccaaaggtagttaatagcttt

SEQ ID NO: 43 - DNA, 27 bp, Glycine max
  source 1..27: mol_type = genomic DNA
tttagttgcacggtaggtttggccacc

SEQ ID NO: 44 - DNA, 27 bp, Glycine max
  source 1..27: mol_type = genomic DNA
tttagtaagaccatgaaataatcggcc

SEQ ID NO: 45 - DNA, 27 bp, Glycine max
  source 1..27: mol_type = genomic DNA
tttaggtgcaccgtgggaaggtacttt

SEQ ID NO: 46 - DNA, 27 bp, Glycine max
  source 1..27: mol_type = genomic DNA
tttggtggaaggacgtacttgacggcg

SEQ ID NO: 47 - DNA, 27 bp, Glycine max
  source 1..27: mol_type = genomic DNA
tttcgaaatggggcgatcaaaggcagc

SEQ ID NO: 48 - DNA, 27 bp, Glycine max
  source 1..27: mol_type = genomic DNA
tttcgggaattgcttgctggggcacca

SEQ ID NO: 49 - DNA, 27 bp, Glycine max
  source 1..27: mol_type = genomic DNA
tttggagagagaaagtacacacgttaa

SEQ ID NO: 50 - DNA, 27 bp, Glycine max
  source 1..27: mol_type = genomic DNA
tttgggcatatacagctttggaaactc

SEQ ID NO: 51 - DNA, 27 bp, Glycine max
  source 1..27: mol_type = genomic DNA
tttagagcgttcgtgcgacaagttaac

SEQ ID NO: 52 - DNA, 27 bp, Glycine max
  source 1..27: mol_type = genomic DNA
tttgccactagggttgccctctgccac

SEQ ID NO: 53 - DNA, 27 bp, Glycine max
  source 1..27: mol_type = genomic DNA
tttggcaagatccgctgcctgcttgag

SEQ ID NO: 54 - DNA, 27 bp, Glycine max
  source 1..27: mol_type = genomic DNA
tttagcatatcaatgaacagccgggtg

SEQ ID NO: 55 - DNA, 27 bp, Glycine max
  source 1..27: mol_type = genomic DNA
tttggtcaatggaagctgtcaatatcg

SEQ ID NO: 56 - DNA, 27 bp, Glycine max
  source 1..27: mol_type = genomic DNA
tttggtgtatacgtgtgtgagacaact

SEQ ID NO: 57 - DNA, 27 bp, Glycine max
  source 1..27: mol_type = genomic DNA
tttagtttcacgtcaacgacgtccata

SEQ ID NO: 58 - DNA, 27 bp, Glycine max
  source 1..27: mol_type = genomic DNA
tttcggcggtaagtacaaccgtagaag

SEQ ID NO: 59 - DNA, 27 bp, Glycine max
  source 1..27: mol_type = genomic DNA
tttgatataatagtaacattggcaacc

SEQ ID NO: 60 - DNA, 27 bp, Glycine max
  source 1..27: mol_type = genomic DNA
tttactcaaacacaaattagagtgggc

SEQ ID NO: 61 - DNA, 27 bp, Glycine max
  source 1..27: mol_type = genomic DNA
tttcgaaccaaacaggttgcattgcat

SEQ ID NO: 62 - DNA, 27 bp, Glycine max
  source 1..27: mol_type = genomic DNA
tttggaattacacactgggtcattgac

SEQ ID NO: 63 - DNA, 27 bp, Glycine max
  source 1..27: mol_type = genomic DNA
tttggtacgcggaagcgaaagccctac

SEQ ID NO: 64 - DNA, 27 bp, Glycine max
  source 1..27: mol_type = genomic DNA
tttcgtccaacatactttcctaacatc

SEQ ID NO: 65 - DNA, 27 bp, Glycine max
  source 1..27: mol_type = genomic DNA
tttagaggaagttcctcccttaccaaa

SEQ ID NO: 66 - DNA, 27 bp, Glycine max
  source 1..27: mol_type = genomic DNA
tttgggagtggtggctgggagcattga

SEQ ID NO: 67 - DNA, 27 bp, Glycine max
  source 1..27: mol_type = genomic DNA
tttactatgaataatgtgacgagacac

SEQ ID NO: 68 - DNA, 27 bp, Glycine max
  source 1..27: mol_type = genomic DNA
tttagggtaacatgaattggccaattt

SEQ ID NO: 69 - DNA, 27 bp, Glycine max
  source 1..27: mol_type = genomic DNA
tttaaatctatctagtccaaatggacc

SEQ ID NO: 70 - DNA, 27 bp, Glycine max
  source 1..27: mol_type = genomic DNA
tttggaggagtaagaatactcagcaca

SEQ ID NO: 71 - DNA, 27 bp, Glycine max
  source 1..27: mol_type = genomic DNA
tttggtaataatcgtctctgatcttta

SEQ ID NO: 72 - DNA, 27 bp, Glycine max
  source 1..27: mol_type = genomic DNA
tttggtctcagaaaggcttggtcaacc

SEQ ID NO: 73 - DNA, 27 bp, Glycine max
  source 1..27: mol_type = genomic DNA
tttagaatcgtgtggatcgtggcctcc

SEQ ID NO: 74 - DNA, 27 bp, Glycine max
  source 1..27: mol_type = genomic DNA
tttagagcaatggcagctgcaccactg

SEQ ID NO: 75 - DNA, 27 bp, Glycine max
  source 1..27: mol_type = genomic DNA
tttggttgcagacgtaaatttccacgc

SEQ ID NO: 76 - DNA, 27 bp, Glycine max
  source 1..27: mol_type = genomic DNA
tttggaggaggccacgatccacacgat

SEQ ID NO: 77 - DNA, 27 bp, Glycine max
  source 1..27: mol_type = genomic DNA
tttggggcaagtggttgaccaagcctt

SEQ ID NO: 78 - DNA, 27 bp, Glycine max
  source 1..27: mol_type = genomic DNA
tttggaatacaagactgacgccgaaac

SEQ ID NO: 79 - DNA, 27 bp, Glycine max
  source 1..27: mol_type = genomic DNA
tttcgcctcctcggcatgattggccac

SEQ ID NO: 80 - DNA, 27 bp, Glycine max
  source 1..27: mol_type = genomic DNA
tttagagctaactcaaattcttgccca

SEQ ID NO: 81 - DNA, 27 bp, Glycine max
  source 1..27: mol_type = genomic DNA
tttagcaagacagttgtgtgctttcaa

SEQ ID NO: 82 - DNA, 27 bp, Glycine max
  source 1..27: mol_type = genomic DNA
tttggtactactttaacgtagacaaag

SEQ ID NO: 83 - DNA, 27 bp, Glycine max
  source 1..27: mol_type = genomic DNA
tttagttactgggttgaaaccaatcct

SEQ ID NO: 84 - DNA, 27 bp, Glycine max
  source 1..27: mol_type = genomic DNA
tttagttcctgtagctatcaaaggtcc

SEQ ID NO: 85 - DNA, 27 bp, Glycine max
  source 1..27: mol_type = genomic DNA
tttggatgttgctatcaaccttccaca

SEQ ID NO: 86 - DNA, 27 bp, Glycine max
  source 1..27: mol_type = genomic DNA
tttagcagttatcaatgcagatcatgg

SEQ ID NO: 87 - DNA, 27 bp, Glycine max
  source 1..27: mol_type = genomic DNA
tttcgcatcaagggataattggctgat

SEQ ID NO: 88 - DNA, 27 bp, Glycine max
  source 1..27: mol_type = genomic DNA
tttagcttgctaggattaggaaccagt

SEQ ID NO: 89 - DNA, 27 bp, Glycine max
  source 1..27: mol_type = genomic DNA
tttggtaatattaatatccatgtatac

SEQ ID NO: 90 - DNA, 27 bp, Glycine max
  source 1..27: mol_type = genomic DNA
tttagtatttaactattgcctcctaat

SEQ ID NO: 91 - DNA, 27 bp, Glycine max
  source 1..27: mol_type = genomic DNA
tttagttcctgtagctatcaaactggt

SEQ ID NO: 92 - DNA, 27 bp, Glycine max
  source 1..27: mol_type = genomic DNA
tttgcataaaggactggcggaataggc

SEQ ID NO: 93 - DNA, 27 bp, Glycine max
  source 1..27: mol_type = genomic DNA
tttagggcgtacactgtacggatatat

SEQ ID NO: 94 - DNA, 27 bp, Glycine max
  source 1..27: mol_type = genomic DNA
tttacatatatccgtacagtgtacgcc

SEQ ID NO: 95 - DNA, 27 bp, Glycine max
  source 1..27: mol_type = genomic DNA
tttagagcaatctaggagtcataatca

SEQ ID NO: 96 - DNA, 27 bp, Glycine max
  source 1..27: mol_type = genomic DNA
tttggatgatggttaatctctgattcc

SEQ ID NO: 97 - DNA, 27 bp, Glycine max
  source 1..27: mol_type = genomic DNA
tttggggtttgtatcagtgtgtgagac

SEQ ID NO: 98 - DNA, 27 bp, Glycine max
  source 1..27: mol_type = genomic DNA
tttggtaaattgagggacactcactca

SEQ ID NO: 99 - DNA, 27 bp, Glycine max
  source 1..27: mol_type = genomic DNA
tttggtcaatgacacgtcttccaactt

SEQ ID NO: 100 - DNA, 27 bp, Glycine max
  source 1..27: mol_type = genomic DNA
tttggaatttccatagagagtaataca

SEQ ID NO: 101 - DNA, 27 bp, Glycine max
  source 1..27: mol_type = genomic DNA
tttagacctctatcacacactcaatga

SEQ ID NO: 102 - DNA, 27 bp, Glycine max
  source 1..27: mol_type = genomic DNA
tttcgcccacaccatatccctagggtg

SEQ ID NO: 103 - DNA, 27 bp, Glycine max
  source 1..27: mol_type = genomic DNA
tttggggatgggttgattatgactcct

SEQ ID NO: 104 - DNA, 27 bp, Glycine max
  source 1..27: mol_type = genomic DNA
tttggtggacagttgttgtcatctagc

SEQ ID NO: 105 - DNA, 27 bp, Glycine max
  source 1..27: mol_type = genomic DNA
tttggtggactgattggtcgtcataat

SEQ ID NO: 106 - DNA, 27 bp, Glycine max
  source 1..27: mol_type = genomic DNA
tttggcacgctccacgctgcatatatg

SEQ ID NO: 107 - DNA, 27 bp, Glycine max
  source 1..27: mol_type = genomic DNA
tttggttgagtggacgagtcatgctac

SEQ ID NO: 108 - DNA, 27 bp, Glycine max
  source 1..27: mol_type = genomic DNA
tttcgaatagccctcactcatacaatc

SEQ ID NO: 109 - DNA, 27 bp, Glycine max
  source 1..27: mol_type = genomic DNA
tttaatctgaataaatattgggccacc

SEQ ID NO: 110 - DNA, 27 bp, Glycine max
  source 1..27: mol_type = genomic DNA
tttgcagccagtttaataatgtttccc

SEQ ID NO: 111 - DNA, 27 bp, Glycine max
  source 1..27: mol_type = genomic DNA
tttcgagaaccaaacagaatgggaagc

SEQ ID NO: 112 - DNA, 27 bp, Glycine max
  source 1..27: mol_type = genomic DNA
tttcgagtagagtttgttcggtcggtc

SEQ ID NO: 113 - DNA, 27 bp, Glycine max
  source 1..27: mol_type = genomic DNA
tttagcaattagcgcaatctcctttgt

SEQ ID NO: 114 - DNA, 27 bp, Glycine max
  source 1..27: mol_type = genomic DNA
tttagcccagtgtaaataatacaataa

SEQ ID NO: 115 - DNA, 27 bp, Glycine max
  source 1..27: mol_type = genomic DNA
tttaggattgtatgagtgagggctatt

SEQ ID NO: 116 - DNA, 27 bp, Glycine max
  source 1..27: mol_type = genomic DNA
tttggggacgtttgctcacaacaatgt

SEQ ID NO: 117 - DNA, 27 bp, Glycine max
  source 1..27: mol_type = genomic DNA
tttggtaacaactgtaaatatttattc

SEQ ID NO: 118 - DNA, 27 bp, Glycine max
  source 1..27: mol_type = genomic DNA
tttggttcctatcgcagtaaattgatc

SEQ ID NO: 119 - DNA, 27 bp, Glycine max
  source 1..27: mol_type = genomic DNA
tttggtataatatggcttcggccaccc

SEQ ID NO: 120 - DNA, 27 bp, Glycine max
  source 1..27: mol_type = genomic DNA
tttggttaaagtccgttccggatcagc

SEQ ID NO: 121 - DNA, 27 bp, Glycine max
  source 1..27: mol_type = genomic DNA
tttacgtctattaggtggcccacttac

SEQ ID NO: 122 - DNA, 27 bp, Glycine max
  source 1..27: mol_type = genomic DNA
tttggtgaccgcatgcgacacgcagca

SEQ ID NO: 123 - DNA, 27 bp, Glycine max
  source 1..27: mol_type = genomic DNA
tttggtgcatgtgggaccaaccagatg

SEQ ID NO: 124 - DNA, 27 bp, Glycine max
  source 1..27: mol_type = genomic DNA
tttggagaaatgaaggaaggggacgaa

SEQ ID NO: 125 - DNA, 27 bp, Glycine max
  source 1..27: mol_type = genomic DNA
tttcgagggcgagacactaaatctgac

SEQ ID NO: 126 - DNA, 27 bp, Glycine max
  source 1..27: mol_type = genomic DNA
tttagcatcactggatttgtctccttt

SEQ ID NO: 127 - DNA, 27 bp, Glycine max
  source 1..27: mol_type = genomic DNA
tttggggctggctatgacccaagaata

SEQ ID NO: 128 - DNA, 27 bp, Glycine max
  source 1..27: mol_type = genomic DNA
tttggtggttgttgaaagcgaaggcca

SEQ ID NO: 129 - DNA, 27 bp, Glycine max
  source 1..27: mol_type = genomic DNA
tttagttaaagagaatgcaaccactat

SEQ ID NO: 130 - DNA, 27 bp, Glycine max
  source 1..27: mol_type = genomic DNA
tttggttgaatgccctcaccattcaca

SEQ ID NO: 131 - DNA, 27 bp, Glycine max
  source 1..27: mol_type = genomic DNA
tttaacaacaaacggaagaaagatcac

SEQ ID NO: 132 - DNA, 27 bp, Glycine max
  source 1..27: mol_type = genomic DNA
tttggacaccggttgcagtagttgaag

SEQ ID NO: 133 - DNA, 27 bp, Glycine max
  source 1..27: mol_type = genomic DNA
tttggattgcgtaggtggaggttctgt

SEQ ID NO: 134 - DNA, 27 bp, Glycine max
  source 1..27: mol_type = genomic DNA
tttagcaccgcaccatgatgattgaca

SEQ ID NO: 135 - DNA, 27 bp, Glycine max
  source 1..27: mol_type = genomic DNA
tttggtgtgttggaatccgaaggaagt

SEQ ID NO: 136 - DNA, 27 bp, Glycine max
  source 1..27: mol_type = genomic DNA
tttacaggctctggcatatacccacat

SEQ ID NO: 137 - DNA, 27 bp, Glycine max
  source 1..27: mol_type = genomic DNA
tttagcaacagactagtctttgactta

SEQ ID NO: 138 - DNA, 27 bp, Glycine max
  source 1..27: mol_type = genomic DNA
tttagctgcaactatctcagtttctga

SEQ ID NO: 139 - DNA, 27 bp, Glycine max
  source 1..27: mol_type = genomic DNA
tttggggaattcaatctcgatggacag

SEQ ID NO: 140 - DNA, 27 bp, Glycine max
  source 1..27: mol_type = genomic DNA
tttggtaggtaaggaattaaggggaag

SEQ ID NO: 141 - DNA, 27 bp, Glycine max
  source 1..27: mol_type = genomic DNA
tttggtcatcaatgacccaataaatca

SEQ ID NO: 142 - DNA, 27 bp, Glycine max
  source 1..27: mol_type = genomic DNA
tttggtttggattgcgtaggtggaggt

SEQ ID NO: 143 - DNA, 27 bp, Glycine max
  source 1..27: mol_type = genomic DNA
tttggtggaggaagctttgttaacaac

SEQ ID NO: 144 - DNA, 27 bp, Glycine max
  source 1..27: mol_type = genomic DNA
tttggagagtgagtaggctatgagaaa

SEQ ID NO: 145 - DNA, 27 bp, Glycine max
  source 1..27: mol_type = genomic DNA
tttggataagcacacgtctctttcatc

SEQ ID NO: 146 - DNA, 27 bp, Glycine max
  source 1..27: mol_type = genomic DNA
tttgggtattaactagctagtacgtac

SEQ ID NO: 147 - DNA, 27 bp, Glycine max
  source 1..27: mol_type = genomic DNA
tttggtaaagagaaagcatgataaact

SEQ ID NO: 148 - DNA, 27 bp, Glycine max
  source 1..27: mol_type = genomic DNA
tttagttataattagttccagtttacc

SEQ ID NO: 149 - DNA, 27 bp, Glycine max
  source 1..27: mol_type = genomic DNA
tttggtcacgcatcgcagtgccattgc

SEQ ID NO: 150 - DNA, 27 bp, Glycine max
  source 1..27: mol_type = genomic DNA
tttagaaactgctggaccggttagttg

SEQ ID NO: 151 - DNA, 27 bp, Glycine max
  source 1..27: mol_type = genomic DNA
tttagacaatgtggagctgatccagtt

SEQ ID NO: 152 - DNA, 27 bp, Glycine max
  source 1..27: mol_type = genomic DNA
tttggacatattgtggaaatagctaat

SEQ ID NO: 153 - DNA, 27 bp, Glycine max
  source 1..27: mol_type = genomic DNA
tttcgagcaacacggaggcatgtttgg

SEQ ID NO: 154 - DNA, 27 bp, Glycine max
  source 1..27: mol_type = genomic DNA
tttcgtatatggaaagaacgaaacaac

SEQ ID NO: 155 - DNA, 27 bp, Glycine max
  source 1..27: mol_type = genomic DNA
tttggtgtgtatcaaatctggaaggca

SEQ ID NO: 156 - DNA, 27 bp, Glycine max
  source 1..27: mol_type = genomic DNA
tttggttagaaacaatcaatcacatgt

SEQ ID NO: 157 - DNA, 27 bp, Glycine max
  source 1..27: mol_type = genomic DNA
tttgtatacactcctcatccgggcagc

SEQ ID NO: 158 - DNA, 27 bp, Glycine max
  source 1..27: mol_type = genomic DNA
tttaacaagagaatataaagcaaggcc

SEQ ID NO: 159 - DNA, 27 bp, Glycine max
  source 1..27: mol_type = genomic DNA
tttaactcaaattctacgatcaacacc

SEQ ID NO: 160 - DNA, 27 bp, Glycine max
  source 1..27: mol_type = genomic DNA
tttggaccggacatcatactcattcaa

SEQ ID NO: 161 - DNA, 27 bp, Glycine max
  source 1..27: mol_type = genomic DNA
tttggactaagatccttatcctcgtca

SEQ ID NO: 162 - DNA, 27 bp, Glycine max
  source 1..27: mol_type = genomic DNA
tttagccacttgacaaactcagatgaa

SEQ ID NO: 163 - DNA, 27 bp, Glycine max
  source 1..27: mol_type = genomic DNA
tttagccctttcaactgaaatataaat

SEQ ID NO: 164 - DNA, 27 bp, Glycine max
  source 1..27: mol_type = genomic DNA
tttagtggaattatgtgtaggagggca

SEQ ID NO: 165 - DNA, 27 bp, Glycine max
  source 1..27: mol_type = genomic DNA
tttgtgggtacaggttcaggtagtagc

SEQ ID NO: 166 - DNA, 27 bp, Glycine max
  source 1..27: mol_type = genomic DNA
tttacccataagaagatataggaaagc

SEQ ID NO: 167 - DNA, 27 bp, Glycine max
  source 1..27: mol_type = genomic DNA
tttggaccaattctaagtctctaacaa

SEQ ID NO: 168 - DNA, 27 bp, Glycine max
  source 1..27: mol_type = genomic DNA
tttgggacgactttatacaatctattt

SEQ ID NO: 169 - DNA, 27 bp, Glycine max
  source 1..27: mol_type = genomic DNA
tttagatcgacaaggtttacatgtaag

SEQ ID NO: 170 - DNA, 27 bp, Glycine max
  source 1..27: mol_type = genomic DNA
tttaggtgcatacactatattgcctaa

SEQ ID NO: 171 - DNA, 27 bp, Glycine max
  source 1..27: mol_type = genomic DNA
tttggaattaggagtgaggttgaagtc

SEQ ID NO: 172 - DNA, 27 bp, Glycine max
  source 1..27: mol_type = genomic DNA
tttgggatgagggcggggtgtggaggt

SEQ ID NO: 173 - DNA, 27 bp, Glycine max
  source 1..27: mol_type = genomic DNA
tttagcgccgtacactactgtcttcat

SEQ ID NO: 174 - DNA, 27 bp, Glycine max
  source 1..27: mol_type = genomic DNA
tttaggccaggtcaggtatctggcatt

SEQ ID NO: 175 - DNA, 27 bp, Glycine max
  source 1..27: mol_type = genomic DNA
tttcgtatcatgacttcagatgtgttc

SEQ ID NO: 176 - DNA, 27 bp, Glycine max
  source 1..27: mol_type = genomic DNA
tttcaacgtacctcactaggttcaccc

SEQ ID NO: 177 - DNA, 27 bp, Glycine max
  source 1..27: mol_type = genomic DNA
tttagcgttaatacttcagtacacaca

SEQ ID NO: 178 - DNA, 27 bp, Glycine max
  source 1..27: mol_type = genomic DNA
tttggtcactgttgtagtaagtgcttc

SEQ ID NO: 179 - DNA, 27 bp, Glycine max
  source 1..27: mol_type = genomic DNA
tttaggatggtgagagaatggatgggt

SEQ ID NO: 180 - DNA, 27 bp, Glycine max
  source 1..27: mol_type = genomic DNA
tttgggacagacacaaattccagaatc

SEQ ID NO: 181 - DNA, 27 bp, Glycine max
  source 1..27: mol_type = genomic DNA
tttggatttaagatgcgtaagcttggt

SEQ ID NO: 182 - DNA, 27 bp, Glycine max
  source 1..27: mol_type = genomic DNA
tttagcatttcttgtcaaatgggtgct

SEQ ID NO: 183 - DNA, 27 bp, Glycine max
  source 1..27: mol_type = genomic DNA
tttggctatcggaataactgaaagaat

SEQ ID NO: 184 - DNA, 27 bp, Glycine max
  source 1..27: mol_type = genomic DNA
tttgggtgaataactgaatattctcaa

SEQ ID NO: 185 - DNA, 27 bp, Glycine max
  source 1..27: mol_type = genomic DNA
tttaggtgaataaagacaaagaacttc

SEQ ID NO: 186 - DNA, 27 bp, Glycine max
  source 1..27: mol_type = genomic DNA
tttggcaatgtccgcatccacattcac

SEQ ID NO: 187 - DNA, 27 bp, Glycine max
  source 1..27: mol_type = genomic DNA
tttagcttctatgcaacgagggtaaag

SEQ ID NO: 188 - DNA, 27 bp, Glycine max
  source 1..27: mol_type = genomic DNA
tttgggtatggtgaatgtggatgcgga

SEQ ID NO: 189 - DNA, 27 bp, Glycine max
  source 1..27: mol_type = genomic DNA
tttagccgaagataagtagtttcatta

SEQ ID NO: 190 - DNA, 27 bp, Glycine max
  source 1..27: mol_type = genomic DNA
tttggatccagtgggttgaatataggc

SEQ ID NO: 191 - DNA, 27 bp, Glycine max
  source 1..27: mol_type = genomic DNA
tttggatgaatatctatcccaaattac

SEQ ID NO: 192 - DNA, 27 bp, Glycine max
  source 1..27: mol_type = genomic DNA
tttggtcaagagcgagcaaagaccacc

SEQ ID NO: 193 - DNA, 27 bp, Glycine max
  source 1..27: mol_type = genomic DNA
tttagtcaaggttccgttccgcaggac

SEQ ID NO: 194 - DNA, 27 bp, Glycine max
  source 1..27: mol_type = genomic DNA
tttagaatcccatgatccttcccttcc

SEQ ID NO: 195 - DNA, 27 bp, Glycine max
  source 1..27: mol_type = genomic DNA
tttggcccaaagaaatgagcgttgagg

SEQ ID NO: 196 - DNA, 27 bp, Glycine max
  source 1..27: mol_type = genomic DNA
tttagctgtatccaggctaactccaaa

SEQ ID NO: 197 - DNA, 27 bp, Glycine max
  source 1..27: mol_type = genomic DNA
tttagtagcgcaaagtagcatgcacgt

SEQ ID NO: 198 - DNA, 27 bp, Glycine max
  source 1..27: mol_type = genomic DNA
tttagtaggccctggcagagccaagga

SEQ ID NO: 199 - DNA, 27 bp, Glycine max
  source 1..27: mol_type = genomic DNA
tttaaatgaaagtgtcgaggtccacac

SEQ ID NO: 200 - DNA, 27 bp, Glycine max
  source 1..27: mol_type = genomic DNA
tttggaactgcagcacgagcagttaag

SEQ ID NO: 201 - DNA, 27 bp, Glycine max
  source 1..27: mol_type = genomic DNA
tttggagttagcctggatacagctaaa

SEQ ID NO: 202 - DNA, 27 bp, Glycine max
  source 1..27: mol_type = genomic DNA
tttcgatctgtggctccgcgcgcagat

SEQ ID NO: 203 - DNA, 27 bp, Glycine max
  source 1..27: mol_type = genomic DNA
tttagcttgtctcctcgctagtatatc

SEQ ID NO: 204 - DNA, 27 bp, Glycine max
  source 1..27: mol_type = genomic DNA
tttaaacggactgaatcatgtataggc

SEQ ID NO: 205 - DNA, 27 bp, Glycine max
  source 1..27: mol_type = genomic DNA
tttaccgagacggataatatcttcttc

SEQ ID NO: 206 - DNA, 27 bp, Glycine max
  source 1..27: mol_type = genomic DNA
tttacgcgacgacgccaaagtcactcc

SEQ ID NO: 207 - DNA, 27 bp, Glycine max
  source 1..27: mol_type = genomic DNA
tttagagtctagagacgtgtcatgcaa

SEQ ID NO: 208 - DNA, 27 bp, Glycine max
  source 1..27: mol_type = genomic DNA
tttggcgtcgtcgcgtaaattggggtg

SEQ ID NO: 209 - DNA, 27 bp, Glycine max
  source 1..27: mol_type = genomic DNA
tttgggcaaggaattagcatgctcatt

SEQ ID NO: 210 - DNA, 27 bp, Glycine max
  source 1..27: mol_type = genomic DNA
tttaggtgatgacatctacaatttcac

SEQ ID NO: 211 - DNA, 27 bp, Glycine max
  source 1..27: mol_type = genomic DNA
tttggtactagaaccgtctgagttgag

SEQ ID NO: 212 - DNA, 27 bp, Glycine max
  source 1..27: mol_type = genomic DNA
tttagttcaactctggtatgttactct

SEQ ID NO: 213 - DNA, 27 bp, Glycine max
  source 1..27: mol_type = genomic DNA
tttagaaattggaattagctcacacaa

SEQ ID NO: 214 - DNA, 27 bp, Glycine max
  source 1..27: mol_type = genomic DNA
tttagaatcattccattccattcagtc

SEQ ID NO: 215 - DNA, 27 bp, Glycine max
  source 1..27: mol_type = genomic DNA
tttggactacatgagtactctgaatca

SEQ ID NO: 216 - DNA, 27 bp, Glycine max
  source 1..27: mol_type = genomic DNA
tttggattcgctattgcgataccgggt

SEQ ID NO: 217 - DNA, 27 bp, Glycine max
  source 1..27: mol_type = genomic DNA
tttagattgagaattgaacgaaatgag

SEQ ID NO: 218 - DNA, 27 bp, Glycine max
  source 1..27: mol_type = genomic DNA
tttggcttcgtaacttcaatggagatc

SEQ ID NO: 219 - DNA, 27 bp, Glycine max
  source 1..27: mol_type = genomic DNA
tttgggaatcctaagtcgaaccgaaca

SEQ ID NO: 220 - DNA, 27 bp, Glycine max
  source 1..27: mol_type = genomic DNA
tttcggagtggctcaactcagacggtt

SEQ ID NO: 221 - DNA, 27 bp, Glycine max
  source 1..27: mol_type = genomic DNA
tttggggcttctttccctagaggggta

SEQ ID NO: 222 - DNA, 27 bp, Glycine max
  source 1..27: mol_type = genomic DNA
tttaggtgataggtaggtcttgctttg

SEQ ID NO: 223 - DNA, 27 bp, Glycine max
  source 1..27: mol_type = genomic DNA
tttagtacgcataaagcttctccctct

SEQ ID NO: 224 - DNA, 27 bp, Glycine max
  source 1..27: mol_type = genomic DNA
tttagtatgtagtttattatgcatcgc

SEQ ID NO: 225 - DNA, 27 bp, Glycine max
  source 1..27: mol_type = genomic DNA
tttagttgaatcagcaaagactttgat

SEQ ID NO: 226 - DNA, 27 bp, Glycine max
  source 1..27: mol_type = genomic DNA
tttatagcaatgaggcttccgtgtcac

SEQ ID NO: 227 - DNA, 27 bp, Glycine max
  source 1..27: mol_type = genomic DNA
tttatgttgataggtacacccagaccc

SEQ ID NO: 228 - DNA, 27 bp, Glycine max
  source 1..27: mol_type = genomic DNA
tttagcaacggccacgacctactcctc

SEQ ID NO: 229 - DNA, 27 bp, Glycine max
  source 1..27: mol_type = genomic DNA
tttgggcaaagaaaggtcttgaattgc

SEQ ID NO: 230 - DNA, 27 bp, Glycine max
  source 1..27: mol_type = genomic DNA
tttgggagaatctttggtggatgttgc

SEQ ID NO: 231 - DNA, 27 bp, Glycine max
  source 1..27: mol_type = genomic DNA
tttgggaagggcgtagggtatggttgc

SEQ ID NO: 232 - DNA, 27 bp, Glycine max
  source 1..27: mol_type = genomic DNA
tttagctgaacaacgtctaggatgtat

SEQ ID NO: 233 - DNA, 27 bp, Glycine max
  source 1..27: mol_type = genomic DNA
tttggtcacatcaagtgcgacaggtat

SEQ ID NO: 234 - DNA, 27 bp, Glycine max
  source 1..27: mol_type = genomic DNA
tttggacacgtgtcagcatccgagaag

SEQ ID NO: 235 - DNA, 27 bp, Glycine max
  source 1..27: mol_type = genomic DNA
tttagaggcagcccatatattcacttg

SEQ ID NO: 236 - DNA, 27 bp, Glycine max
  source 1..27: mol_type = genomic DNA
tttaggcacagaagtttaaggaacttt

SEQ ID NO: 237 - DNA, 27 bp, Glycine max
  source 1..27: mol_type = genomic DNA
tttggggtgttgctctcaggagtgatt

SEQ ID NO: 238 - DNA, 27 bp, Glycine max
  source 1..27: mol_type = genomic DNA
tttggacaccctcacaaacaatacaat

SEQ ID NO: 239 - DNA, 27 bp, Glycine max
  source 1..27: mol_type = genomic DNA
tttcgacagcgacgacaaagacaacgt

SEQ ID NO: 240 - DNA, 27 bp, Glycine max
  source 1..27: mol_type = genomic DNA
tttggatttgacggagaggcaggtgag

SEQ ID NO: 241 - DNA, 27 bp, Glycine max
  source 1..27: mol_type = genomic DNA
tttagcatatccgaatatagctccgca

SEQ ID NO: 242 - DNA, 27 bp, Glycine max
  source 1..27: mol_type = genomic DNA
tttggtgattctctaattgccatccct

SEQ ID NO: 243 - DNA, 27 bp, Glycine max
  source 1..27: mol_type = genomic DNA
tttagtgtcactggctagcaactgatt

SEQ ID NO: 244 - DNA, 27 bp, Glycine max
  source 1..27: mol_type = genomic DNA
tttagaaactctaacaactcggttaaa

SEQ ID NO: 245 - DNA, 27 bp, Glycine max
  source 1..27: mol_type = genomic DNA
tttggtgaatggtggggtacagttata

SEQ ID NO: 246 - DNA, 27 bp, Glycine max
  source 1..27: mol_type = genomic DNA
tttggtggatgttgcatgatgagttgt

SEQ ID NO: 247 - DNA, 27 bp, Glycine max
  source 1..27: mol_type = genomic DNA
tttggtgtgaactaagagtgtagtatt

SEQ ID NO: 248 - DNA, 27 bp, Glycine max
  source 1..27: mol_type = genomic DNA
tttggttgaatatgtttaggggcggaa

SEQ ID NO: 249 - DNA, 27 bp, Glycine max
  source 1..27: mol_type = genomic DNA
tttggagccaataaccgtgatacattc

SEQ ID NO: 250 - DNA, 27 bp, Glycine max
  source 1..27: mol_type = genomic DNA
tttggagaaataagatacaagcaattc

SEQ ID NO: 251 - DNA, 27 bp, Glycine max
  source 1..27: mol_type = genomic DNA
tttagagagaagttaaatggaagcatc

SEQ ID NO: 252 - DNA, 27 bp, Glycine max
  source 1..27: mol_type = genomic DNA
tttagtccatccctaaggaggcaacca

SEQ ID NO: 253 - DNA, 27 bp, Glycine max
  source 1..27: mol_type = genomic DNA
tttggggctccggtccattaaataagc

SEQ ID NO: 254 - DNA, 27 bp, Glycine max
  source 1..27: mol_type = genomic DNA
tttgggaattcctttaacaggtaaggc

SEQ ID NO: 255 - DNA, 27 bp, Glycine max
  source 1..27: mol_type = genomic DNA
tttagtaaatttaacacgcaccgtacc

SEQ ID NO: 256 - DNA, 27 bp, Glycine max
  source 1..27: mol_type = genomic DNA
tttggtgccaggttatgaatggaaagt

SEQ ID NO: 257 - DNA, 27 bp, Glycine max
  source 1..27: mol_type = genomic DNA
tttggaatgggcaaagatccgacatgt

SEQ ID NO: 258 - DNA, 27 bp, Glycine max
  source 1..27: mol_type = genomic DNA
tttagggaaggatagatataacatttc

SEQ ID NO: 259 - DNA, 27 bp, Glycine max
  source 1..27: mol_type = genomic DNA
tttagaattgtgatatatcacctcatc

SEQ ID NO: 260 - DNA, 27 bp, Glycine max
  source 1..27: mol_type = genomic DNA
tttagacaacataataatacgaattgc

SEQ ID NO: 261 - DNA, 27 bp, Glycine max
  source 1..27: mol_type = genomic DNA
tttggcatcctagatttggctccactt

SEQ ID NO: 262 - DNA, 27 bp, Glycine max
  source 1..27: mol_type = genomic DNA
tttaggagccttacctgttaaaggaat

SEQ ID NO: 263 - DNA, 27 bp, Glycine max
  source 1..27: mol_type = genomic DNA
tttggacatatatgctatatcattctc

SEQ ID NO: 264 - DNA, 27 bp, Glycine max
  source 1..27: mol_type = genomic DNA
tttggggtggccaccacatataccaac

SEQ ID NO: 265 - DNA, 27 bp, Glycine max
  source 1..27: mol_type = genomic DNA
tttgggcacctgctctatcatgcagac

SEQ ID NO: 266 - DNA, 27 bp, Glycine max
  source 1..27: mol_type = genomic DNA
tttggctgctgcagacaccgttaagtt

SEQ ID NO: 267 - DNA, 27 bp, Glycine max
  source 1..27: mol_type = genomic DNA
tttggctgggttcctggggaagtcaca

SEQ ID NO: 268 - DNA, 27 bp, Glycine max
  source 1..27: mol_type = genomic DNA
tttggtctaataataatttggggtggc

SEQ ID NO: 269 - DNA, 27 bp, Glycine max
  source 1..27: mol_type = genomic DNA
tttggatgacaactatcagtaacaccg

SEQ ID NO: 270 - DNA, 27 bp, Glycine max
  source 1..27: mol_type = genomic DNA
tttaggcagccatggtgaaggaatatt

SEQ ID NO: 271 - DNA, 27 bp, Glycine max
  source 1..27: mol_type = genomic DNA
tttgatcaaagctaaagcataagcatc

SEQ ID NO: 272 - DNA, 27 bp, Glycine max
  source 1..27: mol_type = genomic DNA
tttagaagttgcatgatgattgtcggt

SEQ ID NO: 273 - DNA, 27 bp, Glycine max
  source 1..27: mol_type = genomic DNA
tttggcaatgttagttggagtgcaact

SEQ ID NO: 274 - DNA, 27 bp, Glycine max
  source 1..27: mol_type = genomic DNA
tttggctccgctacccagataaatgaa

SEQ ID NO: 275 - DNA, 27 bp, Glycine max
  source 1..27: mol_type = genomic DNA
tttacaaggatcacagcaccctcagac

SEQ ID NO: 276 - DNA, 27 bp, Glycine max
  source 1..27: mol_type = genomic DNA
tttaggagctttggggcctcgatttac

SEQ ID NO: 277 - DNA, 27 bp, Glycine max
  source 1..27: mol_type = genomic DNA
tttagcatctatgacaccatgagtatc

SEQ ID NO: 278 - DNA, 27 bp, Glycine max
  source 1..27: mol_type = genomic DNA
tttagcatggctcatcaattgccagct

SEQ ID NO: 279 - DNA, 27 bp, Glycine max
  source 1..27: mol_type = genomic DNA
tttagctatattcagcaaagccctcca

SEQ ID NO: 280 - DNA, 27 bp, Glycine max
  source 1..27: mol_type = genomic DNA
tttgggtcaagaagatgtatgggtgaa

SEQ ID NO: 281 - DNA, 27 bp, Glycine max
  source 1..27: mol_type = genomic DNA
tttggacttagcataaactgaggtgag

SEQ ID NO: 282 - DNA, 27 bp, Glycine max
  source 1..27: mol_type = genomic DNA
tttggagtctagtaagcttctaaagac

SEQ ID NO: 283 - DNA, 27 bp, Glycine max
  source 1..27: mol_type = genomic DNA
tttgggaggtcacgaagatgcttataa

SEQ ID NO: 284 - DNA, 27 bp, Glycine max
  source 1..27: mol_type = genomic DNA
tttagggagctggggtgttgcatttaa

SEQ ID NO: 285 - DNA, 27 bp, Glycine max
  source 1..27: mol_type = genomic DNA
tttaggtatactaatgagtatgctcca

SEQ ID NO: 286 - DNA, 27 bp, Glycine max
  source 1..27: mol_type = genomic DNA
tttcgatgcagatctggacttgggtga

SEQ ID NO: 287 - DNA, 27 bp, Glycine max
  source 1..27: mol_type = genomic DNA
tttgggccagcgaaataaatagttaat

SEQ ID NO: 288 - DNA, 27 bp, Glycine max
  source 1..27: mol_type = genomic DNA
tttgggcacagcaccaaccctagtgac

SEQ ID NO: 289 - DNA, 27 bp, Glycine max
  source 1..27: mol_type = genomic DNA
tttgggtacacccagtagcgtggtcct

SEQ ID NO: 290 - DNA, 27 bp, Glycine max
  source 1..27: mol_type = genomic DNA
tttggctacaaaggcacccagtccaca

SEQ ID NO: 291 - DNA, 27 bp, Glycine max
  source 1..27: mol_type = genomic DNA
tttagtttaatcctctagtacttctcc

SEQ ID NO: 292 - DNA, 27 bp, Glycine max
  source 1..27: mol_type = genomic DNA
tttagctcttctgtcttggaggatacc

SEQ ID NO: 293 - DNA, 27 bp, Glycine max
  source 1..27: mol_type = genomic DNA
tttggagtaagcaatgtgcaggtcaag

SEQ ID NO: 294 - DNA, 27 bp, Glycine max
  source 1..27: mol_type = genomic DNA
tttagatgggctatcagcacagcagct

SEQ ID NO: 295 - DNA, 27 bp, Glycine max
  source 1..27: mol_type = genomic DNA
tttggaaccaagtgtacgtgatctata

SEQ ID NO: 296 - DNA, 27 bp, Glycine max
  source 1..27: mol_type = genomic DNA
tttgggcctaatgggcctcaatttaca

SEQ ID NO: 297 - DNA, 27 bp, Glycine max
  source 1..27: mol_type = genomic DNA
tttggacacttgctatattcgctccct

SEQ ID NO: 298 - DNA, 27 bp, Glycine max
  source 1..27: mol_type = genomic DNA
tttggagttcttgctctcggggaagct

SEQ ID NO: 299 - DNA, 27 bp, Glycine max
  source 1..27: mol_type = genomic DNA
tttagggtagcacaaccttaactcatt

SEQ ID NO: 300 - DNA, 27 bp, Glycine max
  source 1..27: mol_type = genomic DNA
tttggttgacaccataaccctattcgc

SEQ ID NO: 301 - DNA, 27 bp, Glycine max
  source 1..27: mol_type = genomic DNA
tttaacgcaaacgcttagtgatttaac

SEQ ID NO: 302 - DNA, 27 bp, Glycine max
  source 1..27: mol_type = genomic DNA
tttaatgcaaagaataaggcttggcac

SEQ ID NO: 303 - DNA, 27 bp, Glycine max
  source 1..27: mol_type = genomic DNA
tttgcatggatatatagcttctgcagc

SEQ ID NO: 304 - DNA, 27 bp, Glycine max
  source 1..27: mol_type = genomic DNA
tttacggaaagggccgtgtggactggg

SEQ ID NO: 305 - DNA, 27 bp, Glycine max
  source 1..27: mol_type = genomic DNA
tttagatttctgtatgtgggacaattc

SEQ ID NO: 306 - DNA, 27 bp, Glycine max
  source 1..27: mol_type = genomic DNA
tttaggtcaaatttggttgacaccata

SEQ ID NO: 307 - DNA, 27 bp, Glycine max
  source 1..27: mol_type = genomic DNA
tttggaggaacaattgtgcatttagcc

SEQ ID NO: 308 - DNA, 27 bp, Glycine max
  source 1..27: mol_type = genomic DNA
tttgggccgggtggaaacaatggagcc

SEQ ID NO: 309 - DNA, 27 bp, Glycine max
  source 1..27: mol_type = genomic DNA
tttgggaccaagccacctactaggcta

SEQ ID NO: 310 - DNA, 27 bp, Glycine max
  source 1..27: mol_type = genomic DNA
tttggagacaactaaacatgaagcgtg

SEQ ID NO: 311 - DNA, 27 bp, Glycine max
  source 1..27: mol_type = genomic DNA
tttagaaacaagttcgacttccggatg

SEQ ID NO: 312 - DNA, 27 bp, Glycine max
  source 1..27: mol_type = genomic DNA
tttaggatgaggagaactcttaaagtt

SEQ ID NO: 313 - DNA, 27 bp, Glycine max
  source 1..27: mol_type = genomic DNA
tttggttgtggggttgcaaatgtaaac

SEQ ID NO: 314 - DNA, 27 bp, Glycine max
  source 1..27: mol_type = genomic DNA
tttggaacggactacacatgacttaag

SEQ ID NO: 315 - DNA, 27 bp, Glycine max
  source 1..27: mol_type = genomic DNA
tttggcgtttgcaaatactcttctaat

SEQ ID NO: 316 - DNA, 27 bp, Glycine max
  source 1..27: mol_type = genomic DNA
tttcgtccaatttaaacaagccctcag

SEQ ID NO: 317 - DNA, 27 bp, Glycine max
  source 1..27: mol_type = genomic DNA
tttagtgaaataacttgtcgaatatcg

SEQ ID NO: 318 - DNA, 27 bp, Glycine max
  source 1..27: mol_type = genomic DNA
tttaggtacaaagtattaagctgcatc

SEQ ID NO: 319 - DNA, 27 bp, Glycine max
  source 1..27: mol_type = genomic DNA
tttacattgaatgagattggctaggac

SEQ ID NO: 320 - DNA, 27 bp, Glycine max
  source 1..27: mol_type = genomic DNA
tttagggttgttgacgatatgaatcac

SEQ ID NO: 321 - DNA, 27 bp, Glycine max
  source 1..27: mol_type = genomic DNA
tttggtaatagcacaccacaatgtgtt

SEQ ID NO: 322 - DNA, 27 bp, Glycine max
  source 1..27: mol_type = genomic DNA
tttaggctgaataagcagaaaggaatc

SEQ ID NO: 323 - DNA, 27 bp, Glycine max
  source 1..27: mol_type = genomic DNA
tttggaagtatcacatcctgtttattc

SEQ ID NO: 324 - DNA, 27 bp, Glycine max
  source 1..27: mol_type = genomic DNA
tttggcgtggaggcatcatcacatcac

SEQ ID NO: 325 - DNA, 27 bp, Glycine max
  source 1..27: mol_type = genomic DNA
tttagctccttacagcagaggtagccc

SEQ ID NO: 326 - DNA, 27 bp, Glycine max
  source 1..27: mol_type = genomic DNA
tttagggtggtagcctgcactagtccc

SEQ ID NO: 327 - DNA, 27 bp, Glycine max
  source 1..27: mol_type = genomic DNA
tttagatgtaactacattcgtaactgc

SEQ ID NO: 328 - DNA, 27 bp, Glycine max
  source 1..27: mol_type = genomic DNA
tttgggattatccatgtatgatgttgc

SEQ ID NO: 329 - DNA, 27 bp, Glycine max
  source 1..27: mol_type = genomic DNA
tttgggccgaatttgagctgcaatctg

SEQ ID NO: 330 - DNA, 27 bp, Glycine max
  source 1..27: mol_type = genomic DNA
tttggtgggttggggttggggaccgat

SEQ ID NO: 331 - DNA, 27 bp, Glycine max
  source 1..27: mol_type = genomic DNA
tttggtttgacatagtagttgtaaagc

SEQ ID NO: 332 - DNA, 27 bp, Glycine max
  source 1..27: mol_type = genomic DNA
tttggcggagtcgagtctgagtacatg

SEQ ID NO: 333 - DNA, 27 bp, Glycine max
  source 1..27: mol_type = genomic DNA
tttggatccaacaggacagatcaagaa

SEQ ID NO: 334 - DNA, 27 bp, Glycine max
  source 1..27: mol_type = genomic DNA
tttcgtagatctccactcaacgtatac

SEQ ID NO: 335 - DNA, 27 bp, Glycine max
  source 1..27: mol_type = genomic DNA
tttcgttcataacagtgagggaacggc

SEQ ID NO: 336 - DNA, 27 bp, Glycine max
  source 1..27: mol_type = genomic DNA
tttcgttgatggcgtcacaagcaagcc

SEQ ID NO: 337 - DNA, 27 bp, Glycine max
  source 1..27: mol_type = genomic DNA
tttaggaacaagattgggaatgggtca

SEQ ID NO: 338 - DNA, 27 bp, Glycine max
  source 1..27: mol_type = genomic DNA
tttggttgcatttgcatggataaacct

SEQ ID NO: 339 - DNA, 27 bp, Glycine max
  source 1..27: mol_type = genomic DNA
tttaccaagagtttataacaggatgac

SEQ ID NO: 340 - DNA, 27 bp, Glycine max
  source 1..27: mol_type = genomic DNA
tttagaaatagaaggttccctcttgtg

SEQ ID NO: 341 - DNA, 27 bp, Glycine max
  source 1..27: mol_type = genomic DNA
tttagcctatatgatggacacatgtat

SEQ ID NO: 342 - DNA, 27 bp, Glycine max
  source 1..27: mol_type = genomic DNA
tttgggcatacagtatctaataactta

SEQ ID NO: 343 - DNA, 27 bp, Glycine max
  source 1..27: mol_type = genomic DNA
tttagtctcagtaatgaagtttgaact

SEQ ID NO: 344 - DNA, 27 bp, Glycine max
  source 1..27: mol_type = genomic DNA
tttggcttcagctaagaagggacgaga

SEQ ID NO: 345 - DNA, 27 bp, Glycine max
  source 1..27: mol_type = genomic DNA
tttgggcctttgggcctaccataatgg

SEQ ID NO: 346 - DNA, 27 bp, Glycine max
  source 1..27: mol_type = genomic DNA
tttagtgtagataaggtatcaggtcac

SEQ ID NO: 347 - DNA, 27 bp, Glycine max
  source 1..27: mol_type = genomic DNA
tttgcatacacgcgtacaattgaaatc

SEQ ID NO: 348 - DNA, 27 bp, Glycine max
  source 1..27: mol_type = genomic DNA
tttggacaagtactacactgaacatct

SEQ ID NO: 349 - DNA, 27 bp, Glycine max
  source 1..27: mol_type = genomic DNA
tttagatgcgcatagatacagaattgc

SEQ ID NO: 350 - DNA, 27 bp, Glycine max
  source 1..27: mol_type = genomic DNA
tttgggcctaccataatggaaattatt

SEQ ID NO: 351 - DNA, 27 bp, Glycine max
  source 1..27: mol_type = genomic DNA
tttaggctaaatagacatcaacacttg

SEQ ID NO: 352 - DNA, 27 bp, Glycine max
  source 1..27: mol_type = genomic DNA
tttggataaattacatctactgagcat

SEQ ID NO: 353 - DNA, 27 bp, Glycine max
  source 1..27: mol_type = genomic DNA
tttagcatgataaatttgtgagaacta

SEQ ID NO: 354 - DNA, 27 bp, Glycine max
  source 1..27: mol_type = genomic DNA
tttagttgaaattaatgctcagtagat

SEQ ID NO: 355 - DNA, 27 bp, Glycine max
  source 1..27: mol_type = genomic DNA
tttagtgcgcattcgctacgtactcgc

SEQ ID NO: 356 - DNA, 27 bp, Glycine max
  source 1..27: mol_type = genomic DNA
tttggagcatgggctgtgtaagacttg

SEQ ID NO: 357 - DNA, 27 bp, Glycine max
  source 1..27: mol_type = genomic DNA
tttgggtgtatacatgccacacatgat

SEQ ID NO: 358 - DNA, 27 bp, Glycine max
  source 1..27: mol_type = genomic DNA
tttggtacactgaacatgcaatccacc

SEQ ID NO: 359 - DNA, 27 bp, Glycine max
  source 1..27: mol_type = genomic DNA
tttaagagaaggggtcagtataatccc

SEQ ID NO: 360 - DNA, 27 bp, Glycine max
  source 1..27: mol_type = genomic DNA
tttggtgtaccagtggaaggacaagag

SEQ ID NO: 361 - DNA, 27 bp, Glycine max
  source 1..27: mol_type = genomic DNA
tttagtgagatgtcatgtaagataagt

SEQ ID NO: 362 - DNA, 27 bp, Glycine max
  source 1..27: mol_type = genomic DNA
tttacacatatccagtagagatttatc

SEQ ID NO: 363 - DNA, 27 bp, Glycine max
  source 1..27: mol_type = genomic DNA
tttggatttagtgtccaaactaataca

SEQ ID NO: 364 - DNA, 27 bp, Glycine max
  source 1..27: mol_type = genomic DNA
tttgggaatttgatatcccatttagtc

SEQ ID NO: 365 - DNA, 27 bp, Glycine max
  source 1..27: mol_type = genomic DNA
tttgggctgaactcgagagaataaccc

SEQ ID NO: 366 - DNA, 27 bp, Glycine max
  source 1..27: mol_type = genomic DNA
tttagaccaacacgacgtcgtgaatat

SEQ ID NO: 367 - DNA, 27 bp, Glycine max
  source 1..27: mol_type = genomic DNA
tttggcacctaacataaactcagctgt

SEQ ID NO: 368 - DNA, 27 bp, Glycine max
  source 1..27: mol_type = genomic DNA
tttagtggcctgggcagtaaataagca

SEQ ID NO: 369 - DNA, 27 bp, Glycine max
  source 1..27: mol_type = genomic DNA
tttgggtttatatgagggtaattaact

SEQ ID NO: 370 - DNA, 27 bp, Glycine max
  source 1..27: mol_type = genomic DNA
tttcgtgctggtcgtatgacacgagat

SEQ ID NO: 371 - DNA, 27 bp, Glycine max
  source 1..27: mol_type = genomic DNA
tttcagcccatagcaagtttagtggcc

SEQ ID NO: 372 - DNA, 27 bp, Glycine max
  source 1..27: mol_type = genomic DNA
tttagaataaccaacaattagtggtta

SEQ ID NO: 373 - DNA, 27 bp, Glycine max
  source 1..27: mol_type = genomic DNA
tttaggtatttatcctaaagagacatt

SEQ ID NO: 374 - DNA, 27 bp, Glycine max
  source 1..27: mol_type = genomic DNA
tttggtcaattgtacgatatgtctcat

SEQ ID NO: 375 - DNA, 27 bp, Glycine max
  source 1..27: mol_type = genomic DNA
tttggtctctcttctaatatcactaat

SEQ ID NO: 376 - DNA, 27 bp, Glycine max
  source 1..27: mol_type = genomic DNA
tttcgtgttgaacaaacacaggtacac

SEQ ID NO: 377 - DNA, 27 bp, Glycine max
  source 1..27: mol_type = genomic DNA
tttgcactgacgcaaacaagaattctc

SEQ ID NO: 378 - DNA, 27 bp, Glycine max
  source 1..27: mol_type = genomic DNA
tttaggggaactccatttgaatttcct

SEQ ID NO: 379 - DNA, 27 bp, Glycine max
  source 1..27: mol_type = genomic DNA
tttagctagtttggtcttgcggtgtag

SEQ ID NO: 380 - DNA, 27 bp, Glycine max
  source 1..27: mol_type = genomic DNA
tttgggacatcgaacagctaataaatt

SEQ ID NO: 381 - DNA, 27 bp, Glycine max
  source 1..27: mol_type = genomic DNA
tttggtcttatctacggctcttgattc

SEQ ID NO: 382 - DNA, 27 bp, Glycine max
  source 1..27: mol_type = genomic DNA
tttcgcctcaatgaaccaagtaaggaa

SEQ ID NO: 383 - DNA, 27 bp, Glycine max
  source 1..27: mol_type = genomic DNA
tttgggaactccatgtgaatttccatg

SEQ ID NO: 384 - DNA, 27 bp, Glycine max
  source 1..27: mol_type = genomic DNA
tttcgtatgaagacgccaattatcaat

SEQ ID NO: 385 - DNA, 27 bp, Glycine max
  source 1..27: mol_type = genomic DNA
tttggtgacacttaattagtacaagga

SEQ ID NO: 386 - DNA, 27 bp, Glycine max
  source 1..27: mol_type = genomic DNA
tttggatcaatttgatctggaagagac

SEQ ID NO: 387 - DNA, 27 bp, Glycine max
  source 1..27: mol_type = genomic DNA
tttagcgaggcgggttaggatttcaac

SEQ ID NO: 388 - DNA, 27 bp, Glycine max
  source 1..27: mol_type = genomic DNA
tttagcctccaaccatgtcaagacttg

SEQ ID NO: 389 - DNA, 27 bp, Glycine max
  source 1..27: mol_type = genomic DNA
tttagaaagtgagctggtattatttcc

SEQ ID NO: 390 - DNA, 27 bp, Glycine max
  source 1..27: mol_type = genomic DNA
tttagaagcgtgagacttgacgtgatg

SEQ ID NO: 391 - DNA, 27 bp, Glycine max
  source 1..27: mol_type = genomic DNA
tttagatcaagatgagtacagaagaaa

SEQ ID NO: 392 - DNA, 27 bp, Glycine max
  source 1..27: mol_type = genomic DNA
tttggcatatcaataaataacatggct

SEQ ID NO: 393 - DNA, 27 bp, Glycine max
  source 1..27: mol_type = genomic DNA
tttagcccagtccaattaagtcgtagg

SEQ ID NO: 394 - DNA, 27 bp, Glycine max
  source 1..27: mol_type = genomic DNA
tttggaaatattaagggattgaggata

SEQ ID NO: 395 - DNA, 27 bp, Glycine max
  source 1..27: mol_type = genomic DNA
tttggaataactaaagtgatcttcgca

SEQ ID NO: 396 - DNA, 27 bp, Glycine max
  source 1..27: mol_type = genomic DNA
tttggttcccttcactctcacaatgcg

SEQ ID NO: 397 - DNA, 27 bp, Glycine max
  source 1..27: mol_type = genomic DNA
tttagttgataacacactcggaccaga

SEQ ID NO: 398 - DNA, 27 bp, Glycine max
  source 1..27: mol_type = genomic DNA
tttgatgtgaactgcatacaccgccgc

SEQ ID NO: 399 - DNA, 27 bp, Glycine max
  source 1..27: mol_type = genomic DNA
tttggattgtgaagctggtaacccgct

SEQ ID NO: 400 - DNA, 27 bp, Glycine max
  source 1..27: mol_type = genomic DNA
tttaacaccattactagtgtggctgcc

SEQ ID NO: 401 - DNA, 27 bp, Glycine max
  source 1..27: mol_type = genomic DNA
tttggaaattaggagtgcagggtacgt

SEQ ID NO: 402 - DNA, 27 bp, Glycine max
  source 1..27: mol_type = genomic DNA
tttggataaacggtaatgtctctttat

SEQ ID NO: 403 - DNA, 27 bp, Glycine max
  source 1..27: mol_type = genomic DNA
tttaggaccaaatttgtacaaacttag

SEQ ID NO: 404 - DNA, 27 bp, Glycine max
  source 1..27: mol_type = genomic DNA
tttagggaacttgtaacaattagagcc

SEQ ID NO: 405 - DNA, 27 bp, Glycine max
  source 1..27: mol_type = genomic DNA
tttaaggtgagaatacacaaataagtc

SEQ ID NO: 406 - DNA, 27 bp, Glycine max
  source 1..27: mol_type = genomic DNA
tttccaagctgtctccggcgcctaatc

SEQ ID NO: 407 - DNA, 27 bp, Glycine max
  source 1..27: mol_type = genomic DNA
tttgctgtaatttgctagtctcgttgc

SEQ ID NO: 408 - DNA, 27 bp, Glycine max
  source 1..27: mol_type = genomic DNA
tttagataacggggctatagtccattg

SEQ ID NO: 409 - DNA, 27 bp, Glycine max
  source 1..27: mol_type = genomic DNA
tttgggaaatatgagttgaatgccttt

SEQ ID NO: 410 - DNA, 27 bp, Glycine max
  source 1..27: mol_type = genomic DNA
tttcggcctcgatgatgaatatataac

SEQ ID NO: 411 - DNA, 27 bp, Glycine max
  source 1..27: mol_type = genomic DNA
tttggtaaatgggaaatagagatgaga

SEQ ID NO: 412 - DNA, 27 bp, Glycine max
  source 1..27: mol_type = genomic DNA
tttggttgctatacgtgtccgcataaa

SEQ ID NO: 413 - DNA, 27 bp, Glycine max
  source 1..27: mol_type = genomic DNA
tttgtaaccaccttgagcaactcgagc

SEQ ID NO: 414 - DNA, 27 bp, Glycine max
  source 1..27: mol_type = genomic DNA
tttactgctagcagccattctctcaac

SEQ ID NO: 415 - DNA, 27 bp, Glycine max
  source 1..27: mol_type = genomic DNA
tttaggactagccaacatcataccata

SEQ ID NO: 416 - DNA, 27 bp, Glycine max
  source 1..27: mol_type = genomic DNA
tttggcgcgtgtcttcttcacgctcaa

SEQ ID NO: 417 - DNA, 27 bp, Glycine max
  source 1..27: mol_type = genomic DNA
tttgggtttatgctatagtagttatct

SEQ ID NO: 418 - DNA, 27 bp, Glycine max
  source 1..27: mol_type = genomic DNA
tttggtagtttactctgtactattacc

SEQ ID NO: 419 - DNA, 27 bp, Glycine max
  source 1..27: mol_type = genomic DNA
tttggcgcgacttcaatgatgccgctc

SEQ ID NO: 420 - DNA, 27 bp, Glycine max
  source 1..27: mol_type = genomic DNA
tttagctcaagtctttggtgtacgtgc

SEQ ID NO: 421 - DNA, 27 bp, Glycine max
  source 1..27: mol_type = genomic DNA
tttggggtcagcttatcacgtaaagac

SEQ ID NO: 422 - DNA, 27 bp, Glycine max
  source 1..27: mol_type = genomic DNA
tttagggtacgcaccctaatctgtctc

SEQ ID NO: 423 - DNA, 27 bp, Glycine max
  source 1..27: mol_type = genomic DNA
tttcgtagaaataacacgtgccgattc

SEQ ID NO: 424 - DNA, 27 bp, Glycine max
  source 1..27: mol_type = genomic DNA
tttggtatgatattgtaaccacgtcac

SEQ ID NO: 425 - DNA, 27 bp, Glycine max
  source 1..27: mol_type = genomic DNA
tttggacaccctccgcaggatctagcg

SEQ ID NO: 426 - DNA, 27 bp, Glycine max
  source 1..27: mol_type = genomic DNA
tttggacacctttgaagaggatccagc

SEQ ID NO: 427 - DNA, 27 bp, Glycine max
  source 1..27: mol_type = genomic DNA
tttgggaggttgtacaaaggatttccc

SEQ ID NO: 428 - DNA, 27 bp, Glycine max
  source 1..27: mol_type = genomic DNA
tttcgtcataacacagagtagttatgc

SEQ ID NO: 429 - DNA, 27 bp, Glycine max
  source 1..27: mol_type = genomic DNA
tttggtgtacgtgcccatccgaaatag

SEQ ID NO: 430 - DNA, 27 bp, Glycine max
  source 1..27: mol_type = genomic DNA
tttagcaggtttcgctagtcaaacagg

SEQ ID NO: 431 - DNA, 27 bp, Glycine max
  source 1..27: mol_type = genomic DNA
tttcgctagtcaaacaggtctgtcacg

SEQ ID NO: 432 - DNA, 27 bp, Glycine max
  source 1..27: mol_type = genomic DNA
tttaggaatgtgcaatgaatgcgtaac

SEQ ID NO: 433 - DNA, 27 bp, Glycine max
  source 1..27: mol_type = genomic DNA
tttcggatgggcacgtacaccaaagac

SEQ ID NO: 434 - DNA, 27 bp, Glycine max
  source 1..27: mol_type = genomic DNA
tttggtgatataaatgtggactctcat

SEQ ID NO: 435 - DNA, 27 bp, Glycine max
  source 1..27: mol_type = genomic DNA
tttaacattatgatgatcggactcacc

SEQ ID NO: 436 - DNA, 27 bp, Glycine max
  source 1..27: mol_type = genomic DNA
tttagacatagttaccacactacttct

SEQ ID NO: 437 - DNA, 27 bp, Glycine max
  source 1..27: mol_type = genomic DNA
tttagtaatcgggatcaatctcaaact

SEQ ID NO: 438 - DNA, 27 bp, Glycine max
  source 1..27: mol_type = genomic DNA
tttagtctttacgtgataagctgaccc

SEQ ID NO: 439 - DNA, 27 bp, Glycine max
  source 1..27: mol_type = genomic DNA
tttagtgggaattagatgtacagagat

SEQ ID NO: 440 - DNA, 27 bp, Glycine max
  source 1..27: mol_type = genomic DNA
tttagttgctccaacttgtatatgacc

SEQ ID NO: 441 - DNA, 27 bp, Glycine max
  source 1..27: mol_type = genomic DNA
tttaaacatacattgtagggtgagtcc

SEQ ID NO: 442 - DNA, 27 bp, Glycine max
  source 1..27: mol_type = genomic DNA
tttgcaacctcctattgctagccaacc

SEQ ID NO: 443 - DNA, 27 bp, Glycine max
  source 1..27: mol_type = genomic DNA
tttgctgttatggtaggttggaaaggc

SEQ ID NO: 444 - DNA, 27 bp, Glycine max
  source 1..27: mol_type = genomic DNA
tttagagagatggttaagttatcatag

SEQ ID NO: 445 - DNA, 27 bp, Glycine max
  source 1..27: mol_type = genomic DNA
tttggagtttggacacctttgaagagg

SEQ ID NO: 446 - DNA, 27 bp, Glycine max
  source 1..27: mol_type = genomic DNA
tttggataggtgtatgtgtgatccaag

SEQ ID NO: 447 - DNA, 27 bp, Glycine max
  source 1..27: mol_type = genomic DNA
tttagtcactttatgttggactgccct

SEQ ID NO: 448 - DNA, 27 bp, Glycine max
  source 1..27: mol_type = genomic DNA
tttagtcttccatcttcatctatatcc

SEQ ID NO: 449 - DNA, 27 bp, Glycine max
  source 1..27: mol_type = genomic DNA
tttagtgcaatattcgtacttccagga

SEQ ID NO: 450 - DNA, 27 bp, Glycine max
  source 1..27: mol_type = genomic DNA
tttagtgtcagtgtcacaagcggacat

SEQ ID NO: 451 - DNA, 27 bp, Glycine max
  source 1..27: mol_type = genomic DNA
tttagaagaactagctagcctaatccg

SEQ ID NO: 452 - DNA, 27 bp, Glycine max
  source 1..27: mol_type = genomic DNA
tttcggtgtgccgccgacgtcgattcc

SEQ ID NO: 453 - DNA, 27 bp, Glycine max
  source 1..27: mol_type = genomic DNA
tttgggttgcagtgcatgtgcagtggc

SEQ ID NO: 454 - DNA, 27 bp, Glycine max
  source 1..27: mol_type = genomic DNA
tttagagtgtatgattgtgtacctacc

SEQ ID NO: 455 - DNA, 27 bp, Glycine max
  source 1..27: mol_type = genomic DNA
tttagcagttagagggcagtcgtagca

SEQ ID NO: 456 - DNA, 27 bp, Glycine max
  source 1..27: mol_type = genomic DNA
tttagggatatatacgtgagtcagagg

SEQ ID NO: 457 - DNA, 27 bp, Glycine max
  source 1..27: mol_type = genomic DNA
tttggactatgttgtgtttcctcgtac

SEQ ID NO: 458 - DNA, 27 bp, Glycine max
  source 1..27: mol_type = genomic DNA
tttggccacaaatttcgatagttcaaa

SEQ ID NO: 459 - DNA, 27 bp, Glycine max
  source 1..27: mol_type = genomic DNA
tttgggtttattatgctctcttggtcc

SEQ ID NO: 460 - DNA, 27 bp, Glycine max
  source 1..27: mol_type = genomic DNA
tttagtttaatcccttagacgataaat

SEQ ID NO: 461 - DNA, 27 bp, Glycine max
  source 1..27: mol_type = genomic DNA
tttccggcaacaacattatgctttcac

SEQ ID NO: 462 - DNA, 27 bp, Glycine max
  source 1..27: mol_type = genomic DNA
tttagaaactccaacgaattattactg

SEQ ID NO: 463 - DNA, 27 bp, Glycine max
  source 1..27: mol_type = genomic DNA
tttagcaaattaaccggctggctttga

SEQ ID NO: 464 - DNA, 27 bp, Glycine max
  source 1..27: mol_type = genomic DNA
tttagtatcagtatgagtctgagatag

SEQ ID NO: 465 - DNA, 27 bp, Glycine max
  source 1..27: mol_type = genomic DNA
tttagtcatgtccgcttgtgacactga

SEQ ID NO: 466 - DNA, 27 bp, Glycine max
  source 1..27: mol_type = genomic DNA
tttggggtggcaaaggaatggagactc

SEQ ID NO: 467 - DNA, 27 bp, Glycine max
  source 1..27: mol_type = genomic DNA
tttagagagacaagaactgtgcccaaa

SEQ ID NO: 468 - DNA, 27 bp, Glycine max
  source 1..27: mol_type = genomic DNA
tttggtgcggcagaaacagaggaagaa

SEQ ID NO: 469 - DNA, 27 bp, Glycine max
  source 1..27: mol_type = genomic DNA
tttagaggtatctaagtagagaagtca

SEQ ID NO: 470 - DNA, 27 bp, Glycine max
  source 1..27: mol_type = genomic DNA
tttgggcacagttcttgtctctctaaa

SEQ ID NO: 471 - DNA, 27 bp, Glycine max
  source 1..27: mol_type = genomic DNA
tttaatccaaatttgcacaagataatc

SEQ ID NO: 472 - DNA, 27 bp, Glycine max
  source 1..27: mol_type = genomic DNA
tttggattaaactttatgggtgcacgt

SEQ ID NO: 473 - DNA, 27 bp, Glycine max
  source 1..27: mol_type = genomic DNA
tttagcataaattatatacaatttaac

SEQ ID NO: 474 - DNA, 27 bp, Glycine max
  source 1..27: mol_type = genomic DNA
tttgggtctatatatatgacaactgaa

SEQ ID NO: 475 - DNA, 27 bp, Glycine max
  source 1..27: mol_type = genomic DNA
tttggtgctaattagatcagattggat

SEQ ID NO: 476 - DNA, 27 bp, Glycine max
  source 1..27: mol_type = genomic DNA
tttaggcaatggggttccgaagaagat

SEQ ID NO: 477 - DNA, 27 bp, Glycine max
  source 1..27: mol_type = genomic DNA
tttagctagaagaacacccaagatgca

SEQ ID NO: 478 - DNA, 27 bp, Glycine max
  source 1..27: mol_type = genomic DNA
tttgggtcttcgtggcagaaggtgtta

SEQ ID NO: 479 - DNA, 27 bp, Glycine max
  source 1..27: mol_type = genomic DNA
tttaccggtttaggcatgtgtacccac

SEQ ID NO: 480 - DNA, 27 bp, Glycine max
  source 1..27: mol_type = genomic DNA
tttgctgcaaactcctttaatagaaac

SEQ ID NO: 481 - DNA, 27 bp, Glycine max
  source 1..27: mol_type = genomic DNA
tttggaaagtaattatgtagccaaatg

SEQ ID NO: 482 - DNA, 27 bp, Glycine max
  source 1..27: mol_type = genomic DNA
tttcgaatgatgaactgaaaccataaa

SEQ ID NO: 483 - DNA, 27 bp, Glycine max
  source 1..27: mol_type = genomic DNA
tttggtgcaaactaacttgctacagaa

SEQ ID NO: 484 - DNA, 27 bp, Glycine max
  source 1..27: mol_type = genomic DNA
tttagaatattggtgtcaagacccggc

SEQ ID NO: 485 - DNA, 27 bp, Glycine max
  source 1..27: mol_type = genomic DNA
tttgctgtgaacacgaatacgggttac

SEQ ID NO: 486 - DNA, 27 bp, Glycine max
  source 1..27: mol_type = genomic DNA
tttagggataatgttctcgatgacgaa

SEQ ID NO: 487 - DNA, 27 bp, Glycine max
  source 1..27: mol_type = genomic DNA
tttaggtactaacaactcatggagaca

SEQ ID NO: 488 - DNA, 27 bp, Glycine max
  source 1..27: mol_type = genomic DNA
tttaggtgacacgcacgaattggtcaa

SEQ ID NO: 489 - DNA, 27 bp, Glycine max
  source 1..27: mol_type = genomic DNA
tttggtgagtatgaaaggaagaagcag

SEQ ID NO: 490 - DNA, 27 bp, Glycine max
  source 1..27: mol_type = genomic DNA
tttagacctcaattagaggagcttaag

SEQ ID NO: 491 - DNA, 27 bp, Glycine max
  source 1..27: mol_type = genomic DNA
tttagtacctaattatgacggaacaaa

SEQ ID NO: 492 - DNA, 27 bp, Glycine max
  source 1..27: mol_type = genomic DNA
tttggtctctcgaatttaataactcat

SEQ ID NO: 493 - DNA, 27 bp, Glycine max
  source 1..27: mol_type = genomic DNA
tttgcactgaagtgtagctcgcacggc

SEQ ID NO: 494 - DNA, 27 bp, Glycine max
  source 1..27: mol_type = genomic DNA
tttagattgaaggtatagataattggc

SEQ ID NO: 495 - DNA, 27 bp, Glycine max
  source 1..27: mol_type = genomic DNA
tttagggtttgaactcagcgcggtccg

SEQ ID NO: 496 - DNA, 27 bp, Glycine max
  source 1..27: mol_type = genomic DNA
tttgctataaacaccaacaatactatc

SEQ ID NO: 497 - DNA, 27 bp, Glycine max
  source 1..27: mol_type = genomic DNA
tttggaaactcacgaggatcaggatag

SEQ ID NO: 498 - DNA, 27 bp, Glycine max
  source 1..27: mol_type = genomic DNA
tttaggtgtggaagttgtggcagtaga

SEQ ID NO: 499 - DNA, 27 bp, Glycine max
  source 1..27: mol_type = genomic DNA
tttagtactggttatagctgcagccct

SEQ ID NO: 500 - DNA, 27 bp, Glycine max
  source 1..27: mol_type = genomic DNA
tttgaacgcaaatcttatcgtaacaac

SEQ ID NO: 501 - DNA, 27 bp, Glycine max
  source 1..27: mol_type = genomic DNA
tttgcgttcaaattacaagatgtgacc

SEQ ID NO: 502 - DNA, 27 bp, Glycine max
  source 1..27: mol_type = genomic DNA
tttgggatcatgtactgtgtcttataa

SEQ ID NO: 503 - DNA, 27 bp, Glycine max
  source 1..27: mol_type = genomic DNA
tttaggtacactggaggtgcaagacac

SEQ ID NO: 504 - DNA, 27 bp, Glycine max
  source 1..27: mol_type = genomic DNA
tttgggacaataatatgactgagctcc

SEQ ID NO: 505 - DNA, 27 bp, Glycine max
  source 1..27: mol_type = genomic DNA
tttacaatgacagatatgtaggccatc

SEQ ID NO: 506 - DNA, 27 bp, Glycine max
  source 1..27: mol_type = genomic DNA
tttggatccgtagctactccgatctct

SEQ ID NO: 507 - DNA, 27 bp, Glycine max
  source 1..27: mol_type = genomic DNA
tttggcagctaagcaatggagctgctg

SEQ ID NO: 508 - DNA, 27 bp, Glycine max
  source 1..27: mol_type = genomic DNA
tttagtaccacactcctgcataaatat

SEQ ID NO: 509 - DNA, 27 bp, Glycine max
  source 1..27: mol_type = genomic DNA
tttcctcaaacgacgtaccggatttcc

SEQ ID NO: 510 - DNA, 27 bp, Glycine max
  source 1..27: mol_type = genomic DNA
tttagaattctcccgaagttacatcac

SEQ ID NO: 511 - DNA, 27 bp, Glycine max
  source 1..27: mol_type = genomic DNA
tttgagtgtattggccacaaactttcc

SEQ ID NO: 512 - DNA, 27 bp, Glycine max
  source 1..27: mol_type = genomic DNA
tttagaagacagacacatacatggaca

SEQ ID NO: 513 - DNA, 27 bp, Glycine max
  source 1..27: mol_type = genomic DNA
tttggactcatcggaacgtgatcacac

SEQ ID NO: 514 - DNA, 27 bp, Glycine max
  source 1..27: mol_type = genomic DNA
tttcgccgaaatccggtgaaacgggct

SEQ ID NO: 515 - DNA, 27 bp, Glycine max
  source 1..27: mol_type = genomic DNA
tttcggggtggcggagacaggagaacc

SEQ ID NO: 516 - DNA, 27 bp, Glycine max
  source 1..27: mol_type = genomic DNA
tttcgttgtagtagcaatgctattgac

SEQ ID NO: 517 - DNA, 27 bp, Glycine max
  source 1..27: mol_type = genomic DNA
tttagagtcaccacctttcaaattaca

SEQ ID NO: 518 - DNA, 27 bp, Glycine max
  source 1..27: mol_type = genomic DNA
tttagtcttacccatctcattaacact

SEQ ID NO: 519 - DNA, 27 bp, Glycine max
  source 1..27: mol_type = genomic DNA
tttagaagtgataagacaggagataaa

SEQ ID NO: 520 - DNA, 27 bp, Glycine max
  source 1..27: mol_type = genomic DNA
tttcgactcataccatacatcatacta

SEQ ID NO: 521 - DNA, 27 bp, Glycine max
  source 1..27: mol_type = genomic DNA
tttggagcatttggggacgtatttatg

SEQ ID NO: 522 - DNA, 27 bp, Glycine max
  source 1..27: mol_type = genomic DNA
tttgggaaatgtctatggtgggaggtt

SEQ ID NO: 523 - DNA, 27 bp, Glycine max
  source 1..27: mol_type = genomic DNA
tttagtacgtgcatgattgggaaggaa

SEQ ID NO: 524 - DNA, 27 bp, Glycine max
  source 1..27: mol_type = genomic DNA
tttggtgcaatagcgagcagcgatggc

SEQ ID NO: 525 - DNA, 27 bp, Glycine max
  source 1..27: mol_type = genomic DNA
tttagacgcatgatagcattcctcatc

SEQ ID NO: 526 - DNA, 27 bp, Glycine max
  source 1..27: mol_type = genomic DNA
tttcgggccatcgctgctcgctattgc

SEQ ID NO: 527 - DNA, 27 bp, Glycine max
  source 1..27: mol_type = genomic DNA
tttcgggccattcgccaagtacctttc

SEQ ID NO: 528 - DNA, 27 bp, Glycine max
  source 1..27: mol_type = genomic DNA
tttgggtcaataggtggcgtgaatgtg

SEQ ID NO: 529 - DNA, 27 bp, Glycine max
  source 1..27: mol_type = genomic DNA
tttggctccacgacagagtttgaggag

SEQ ID NO: 530 - DNA, 27 bp, Glycine max
  source 1..27: mol_type = genomic DNA
tttactgtcaatctctcgtttggctcc

SEQ ID NO: 531 - DNA, 27 bp, Glycine max
  source 1..27: mol_type = genomic DNA
tttggagaagaaagagggtagggtcca

SEQ ID NO: 532 - DNA, 27 bp, Glycine max
  source 1..27: mol_type = genomic DNA
tttcaaatcacatagaagagggccggc

SEQ ID NO: 533 - DNA, 27 bp, Glycine max
  source 1..27: mol_type = genomic DNA
tttcgaaccaaatgcttaggagccaat

SEQ ID NO: 534 - DNA, 27 bp, Glycine max
  source 1..27: mol_type = genomic DNA
tttggaactctaacatgaggcatatat

SEQ ID NO: 535 - DNA, 27 bp, Glycine max
  source 1..27: mol_type = genomic DNA
tttggcgagagtagtatgtactcagtt

SEQ ID NO: 536 - DNA, 27 bp, Glycine max
  source 1..27: mol_type = genomic DNA
tttagctagggacagattcaggattat

SEQ ID NO: 537 - DNA, 27 bp, Glycine max
  source 1..27: mol_type = genomic DNA
tttaggaaggtgcaacgaatgtcgttg

SEQ ID NO: 538 - DNA, 27 bp, Glycine max
  source 1..27: mol_type = genomic DNA
tttacgtacctcgacgtccgtataatc

SEQ ID NO: 539 - DNA, 27 bp, Glycine max
  source 1..27: mol_type = genomic DNA
tttagccattgatgaagggttgatgag

SEQ ID NO: 540 - DNA, 27 bp, Glycine max
  source 1..27: mol_type = genomic DNA
tttgggaatacagaaagttatgtaaga

SEQ ID NO: 541 - DNA, 27 bp, Glycine max
  source 1..27: mol_type = genomic DNA
tttgggtgcaattaattaggctaacga

SEQ ID NO: 542 - DNA, 27 bp, Glycine max
  source 1..27: mol_type = genomic DNA
tttggaccaaacctaccctttgaattc

SEQ ID NO: 543 - DNA, 27 bp, Glycine max
  source 1..27: mol_type = genomic DNA
tttgggataaattatatcaccaaaggc

SEQ ID NO: 544 - DNA, 27 bp, Glycine max
  source 1..27: mol_type = genomic DNA
tttagtatatcacaaggccacgtcaat

SEQ ID NO: 545 - DNA, 27 bp, Glycine max
  source 1..27: mol_type = genomic DNA
tttgctcttatcatctccacgaaacac

SEQ ID NO: 546 - DNA, 27 bp, Glycine max
  source 1..27: mol_type = genomic DNA
tttaataacagtcgtatggtctaacgc

SEQ ID NO: 547 - DNA, 27 bp, Glycine max
  source 1..27: mol_type = genomic DNA
tttgatactacaaagccacgagatgac

SEQ ID NO: 548 - DNA, 27 bp, Glycine max
  source 1..27: mol_type = genomic DNA
tttagggagaatgttcacatgtaggca

SEQ ID NO: 549 - DNA, 27 bp, Glycine max
  source 1..27: mol_type = genomic DNA
tttcgtgacctttgggtggcagtgaac

SEQ ID NO: 550 - DNA, 27 bp, Glycine max
  source 1..27: mol_type = genomic DNA
tttggactatgtgtttcagttagaaac

SEQ ID NO: 551 - DNA, 27 bp, Glycine max
  source 1..27: mol_type = genomic DNA
tttggagtggtgtcaacgtgaatacga

SEQ ID NO: 552 - DNA, 27 bp, Glycine max
  source 1..27: mol_type = genomic DNA
tttagcatttgttatgaacatacggac

SEQ ID NO: 553 - DNA, 27 bp, Glycine max
  source 1..27: mol_type = genomic DNA
tttggtatgagagtttatgaatattcg

SEQ ID NO: 554 - DNA, 27 bp, Glycine max
  source 1..27: mol_type = genomic DNA
tttggagctagtaagagataaatttcc

SEQ ID NO: 555 - DNA, 27 bp, Glycine max
  source 1..27: mol_type = genomic DNA
tttggaacttacctggcataaatactc

SEQ ID NO: 556 - DNA, 27 bp, Glycine max
  source 1..27: mol_type = genomic DNA
tttagctgtaatcaattgaaataccag

SEQ ID NO: 557 - DNA, 27 bp, Glycine max
  source 1..27: mol_type = genomic DNA
tttggtttcatatctcactcccaattt

SEQ ID NO: 558 - DNA, 27 bp, Glycine max
  source 1..27: mol_type = genomic DNA
tttggaaatttatctcttactagctcc

SEQ ID NO: 559 - DNA, 27 bp, Glycine max
  source 1..27: mol_type = genomic DNA
tttcgatgcaagaaaggaagtagtgtg

SEQ ID NO: 560 - DNA, 27 bp, Glycine max
  source 1..27: mol_type = genomic DNA
tttggggacacatgtctatgtgagttg

SEQ ID NO: 561 - DNA, 27 bp, Glycine max
  source 1..27: mol_type = genomic DNA
tttcgttcatgtggcacgtcggtcatg

SEQ ID NO: 562 - DNA, 27 bp, Glycine max
  source 1..27: mol_type = genomic DNA
tttaagtggattcacggtggagggaac

SEQ ID NO: 563 - DNA, 27 bp, Glycine max
  source 1..27: mol_type = genomic DNA
tttggaacaacttaataggggtatcct

SEQ ID NO: 564 - DNA, 27 bp, Glycine max
  source 1..27: mol_type = genomic DNA
tttagaatttgtgcaagtaatccctgc

SEQ ID NO: 565 - DNA, 27 bp, Glycine max
  source 1..27: mol_type = genomic DNA
tttcgaccagatgcagggattacttgc

SEQ ID NO: 566 - DNA, 27 bp, Glycine max
  source 1..27: mol_type = genomic DNA
tttagtagctgaccaagttacccaaca

SEQ ID NO: 567 - DNA, 27 bp, Glycine max
  source 1..27: mol_type = genomic DNA
tttaataatatgtgtcaatctcatacc

SEQ ID NO: 568 - DNA, 27 bp, Glycine max
  source 1..27: mol_type = genomic DNA
tttagttctcttcctaacctcacgcga

SEQ ID NO: 569 - DNA, 27 bp, Glycine max
  source 1..27: mol_type = genomic DNA
tttggtctgataccactactcagctgc

SEQ ID NO: 570 - DNA, 27 bp, Glycine max
  source 1..27: mol_type = genomic DNA
tttagtaggagggacttgacctcgaac

SEQ ID NO: 571 - DNA, 27 bp, Glycine max
  source 1..27: mol_type = genomic DNA
tttggacctacaacatgcattctactc

SEQ ID NO: 572 - DNA, 27 bp, Glycine max
  source 1..27: mol_type = genomic DNA
tttagaagaggttgtggttgggtggac

SEQ ID NO: 573 - DNA, 27 bp, Glycine max
  source 1..27: mol_type = genomic DNA
tttagaaaggaagttgtcgctgaaacc

SEQ ID NO: 574 - DNA, 27 bp, Glycine max
  source 1..27: mol_type = genomic DNA
tttggacatttggtctagtggcacggg

SEQ ID NO: 575 - DNA, 27 bp, Glycine max
  source 1..27: mol_type = genomic DNA
tttggccataaacatcgatctgtgtcg

SEQ ID NO: 576 - DNA, 27 bp, Glycine max
  source 1..27: mol_type = genomic DNA
tttaggcgtgagtataacccggttggt

SEQ ID NO: 577 - DNA, 27 bp, Glycine max
  source 1..27: mol_type = genomic DNA
tttggtggggttgggacgtgcggtggt

SEQ ID NO: 578 - DNA, 27 bp, Glycine max
  source 1..27: mol_type = genomic DNA
tttacgtttacatctggaggtggaaac

SEQ ID NO: 579 - DNA, 27 bp, Glycine max
  source 1..27: mol_type = genomic DNA
tttagtagcatctcaccttgtggagat

SEQ ID NO: 580 - DNA, 27 bp, Glycine max
  source 1..27: mol_type = genomic DNA
tttggtctagtggcacgggtcttgctt

SEQ ID NO: 581 - DNA, 27 bp, Glycine max
  source 1..27: mol_type = genomic DNA
tttggatgcccatcttccttgtgaggt

SEQ ID NO: 582 - DNA, 27 bp, Glycine max
  source 1..27: mol_type = genomic DNA
tttggcgacattataattagaaagaca

SEQ ID NO: 583 - DNA, 27 bp, Glycine max
  source 1..27: mol_type = genomic DNA
tttgggaatagaacactttagtaaaga

SEQ ID NO: 584 - DNA, 27 bp, Glycine max
  source 1..27: mol_type = genomic DNA
tttggggtaggaaagaaagaacaaggg

SEQ ID NO: 585 - DNA, 27 bp, Glycine max
  source 1..27: mol_type = genomic DNA
tttaggtcattaattgcagagcgaaga

SEQ ID NO: 586 - DNA, 27 bp, Glycine max
  source 1..27: mol_type = genomic DNA
tttagtacgagttaaagatgtatcgat

SEQ ID NO: 587 - DNA, 27 bp, Glycine max
  source 1..27: mol_type = genomic DNA
tttcgtccatcccaccatgctcaataa

SEQ ID NO: 588 - DNA, 27 bp, Glycine max
  source 1..27: mol_type = genomic DNA
tttcgataaagtgaaagaacatcaacc

SEQ ID NO: 589 - DNA, 27 bp, Glycine max
  source 1..27: mol_type = genomic DNA
tttggcacagcttgggattcgacgtct

SEQ ID NO: 590 - DNA, 27 bp, Glycine max
  source 1..27: mol_type = genomic DNA
tttagtgtctcaaagcagaatatgcgt

SEQ ID NO: 591 - DNA, 27 bp, Glycine max
  source 1..27: mol_type = genomic DNA
tttgggattaacatctttcgataaagt

SEQ ID NO: 592 - DNA, 27 bp, Glycine max
  source 1..27: mol_type = genomic DNA
tttgcgcacacgtaccagcaaagaacc

SEQ ID NO: 593 - DNA, 27 bp, Glycine max
  source 1..27: mol_type = genomic DNA
tttgggtcggagagaatagtcacaagc

SEQ ID NO: 594 - DNA, 27 bp, Glycine max
  source 1..27: mol_type = genomic DNA
tttacacaaataacacaaagcaatcac

SEQ ID NO: 595 - DNA, 27 bp, Glycine max
  source 1..27: mol_type = genomic DNA
tttagtgtcattgctgacacgattcat

SEQ ID NO: 596 - DNA, 27 bp, Glycine max
  source 1..27: mol_type = genomic DNA
tttgctggtacgtgtgcgcaaatttac

SEQ ID NO: 597 - DNA, 27 bp, Glycine max
  source 1..27: mol_type = genomic DNA
tttggtggaaactacaagcttagtcac

SEQ ID NO: 598 - DNA, 27 bp, Glycine max
  source 1..27: mol_type = genomic DNA
tttggcagcaagtatgatatgggcgtg

SEQ ID NO: 599 - DNA, 27 bp, Glycine max
  source 1..27: mol_type = genomic DNA
tttcgttccatatggaaaggtgttggc

SEQ ID NO: 600 - DNA, 27 bp, Glycine max
  source 1..27: mol_type = genomic DNA
tttacactgatagtgacaggacttaac

SEQ ID NO: 601 - DNA, 27 bp, Glycine max
  source 1..27: mol_type = genomic DNA
tttagtcagccacagtcaactaatgcg

SEQ ID NO: 602 - DNA, 27 bp, Glycine max
  source 1..27: mol_type = genomic DNA
tttactgcctccgtgccacgtgtgatc

SEQ ID NO: 603 - DNA, 27 bp, Glycine max
  source 1..27: mol_type = genomic DNA
tttagctagatcagtacgcaatttaaa

SEQ ID NO: 604 - DNA, 27 bp, Glycine max
  source 1..27: mol_type = genomic DNA
tttagtaactcatggaggattgaattc

SEQ ID NO: 605 - DNA, 27 bp, Glycine max
  source 1..27: mol_type = genomic DNA
tttagtcttcatcatatgtccgcattc

SEQ ID NO: 606 - DNA, 27 bp, Glycine max
  source 1..27: mol_type = genomic DNA
tttggtgcatcgcaagaaatgtatata

SEQ ID NO: 607 - DNA, 27 bp, Glycine max
  source 1..27: mol_type = genomic DNA
tttagttatatacgcatcagcatctat

SEQ ID NO: 608 - DNA, 27 bp, Glycine max
  source 1..27: mol_type = genomic DNA
tttcgaacaatgttgccagagcgtgat

SEQ ID NO: 609 - DNA, 27 bp, Glycine max
  source 1..27: mol_type = genomic DNA
tttcgattatgcatgcactgacaaggc

SEQ ID NO: 610 - DNA, 27 bp, Glycine max
  source 1..27: mol_type = genomic DNA
tttggcaaattggcatagttggggtag

SEQ ID NO: 611 - DNA, 27 bp, Glycine max
  source 1..27: mol_type = genomic DNA
tttcgaagaagataaagtgatgaactc

SEQ ID NO: 612 - DNA, 27 bp, Glycine max
  source 1..27: mol_type = genomic DNA
tttcgcaactgccatcgttggccattc

SEQ ID NO: 613 - DNA, 27 bp, Glycine max
  source 1..27: mol_type = genomic DNA
tttgacacgaccaaacacaatccaaac

SEQ ID NO: 614 - DNA, 27 bp, Glycine max
  source 1..27: mol_type = genomic DNA
tttgtcctaaccatgaaatgggctccc

SEQ ID NO: 615 - DNA, 27 bp, Glycine max
  source 1..27: mol_type = genomic DNA
tttagacgcttgattcagaaacaggat

SEQ ID NO: 616 - DNA, 27 bp, Glycine max
  source 1..27: mol_type = genomic DNA
tttggtgctagaatagaaagcattaga

SEQ ID NO: 617 - DNA, 27 bp, Glycine max
  source 1..27: mol_type = genomic DNA
tttgttctcagtgctgccaacctaagc

SEQ ID NO: 618 - DNA, 27 bp, Glycine max
  source 1..27: mol_type = genomic DNA
tttggaccaatcagagtggaacttatc

SEQ ID NO: 619 - DNA, 27 bp, Glycine max
  source 1..27: mol_type = genomic DNA
tttcgtcaaatccgattcaaactaaac

SEQ ID NO: 620 - DNA, 27 bp, Glycine max
  source 1..27: mol_type = genomic DNA
tttggacggttaacggcaatataatcc

SEQ ID NO: 621 - DNA, 27 bp, Glycine max
  source 1..27: mol_type = genomic DNA
tttggagctacgttggtcagctctttg

SEQ ID NO: 622 - DNA, 27 bp, Glycine max
  source 1..27: mol_type = genomic DNA
tttaggaattgctatgggcacgcacgt

SEQ ID NO: 623 - DNA, 27 bp, Glycine max
  source 1..27: mol_type = genomic DNA
tttggcagtgttggtcgtttgagacgt

SEQ ID NO: 624 - DNA, 27 bp, Glycine max
  source 1..27: mol_type = genomic DNA
tttagtacaagtattgtgttgttggcc

SEQ ID NO: 625 - DNA, 27 bp, Glycine max
  source 1..27: mol_type = genomic DNA
tttgagtataaatcatgggaatgtccc

SEQ ID NO: 626 - DNA, 27 bp, Glycine max
  source 1..27: mol_type = genomic DNA
tttgccagtaggatccttacatcttgc

SEQ ID NO: 627 - DNA, 27 bp, Glycine max
  source 1..27: mol_type = genomic DNA
tttggactgaccatattatataaggag

SEQ ID NO: 628 - DNA, 27 bp, Glycine max
  source 1..27: mol_type = genomic DNA
tttagctagtataagcaactatcaagg

SEQ ID NO: 629 - DNA, 27 bp, Glycine max
  source 1..27: mol_type = genomic DNA
tttaggcatattatgaatcaccgatga

SEQ ID NO: 630 - DNA, 27 bp, Glycine max
  source 1..27: mol_type = genomic DNA
tttggtctctgttacctaggtcctaag

SEQ ID NO: 631 - DNA, 27 bp, Glycine max
  source 1..27: mol_type = genomic DNA
tttcgtgctagttccttgcttcacatc

SEQ ID NO: 632 - DNA, 27 bp, Glycine max
  source 1..27: mol_type = genomic DNA
tttagtggcatgttgatagttgcagag

SEQ ID NO: 633 - DNA, 27 bp, Glycine max
  source 1..27: mol_type = genomic DNA
tttagaaatactcaatctcatatagcc

SEQ ID NO: 634 - DNA, 27 bp, Glycine max
  source 1..27: mol_type = genomic DNA
tttagacagatattaacctaaacatac

SEQ ID NO: 635 - DNA, 27 bp, Glycine max
  source 1..27: mol_type = genomic DNA
tttagcaccaccatccaagattaagtc

SEQ ID NO: 636 - DNA, 27 bp, Glycine max
  source 1..27: mol_type = genomic DNA
tttagcaccatgtattgagagagagtc

SEQ ID NO: 637 - DNA, 27 bp, Glycine max
  source 1..27: mol_type = genomic DNA
tttggaagaaacacttatgtcagtcac

SEQ ID NO: 638 - DNA, 27 bp, Glycine max
  source 1..27: mol_type = genomic DNA
tttagcgagttggaacgacccatgtcc

SEQ ID NO: 639 - DNA, 27 bp, Glycine max
  source 1..27: mol_type = genomic DNA
tttggtgcttaagacaactgggcttgc

SEQ ID NO: 640 - DNA, 27 bp, Glycine max
  source 1..27: mol_type = genomic DNA
tttggaaggtctaaactcctccaagac

SEQ ID NO: 641 - DNA, 27 bp, Glycine max
  source 1..27: mol_type = genomic DNA
tttggactatgtgagcaaggaatgatc

SEQ ID NO: 642 - DNA, 27 bp, Glycine max
  source 1..27: mol_type = genomic DNA
tttggcacgatatggcgatatgaacta

SEQ ID NO: 643 - DNA, 27 bp, Glycine max
  source 1..27: mol_type = genomic DNA
tttagcctgtggtttagaggagaacca

SEQ ID NO: 644 - DNA, 27 bp, Glycine max
  source 1..27: mol_type = genomic DNA
tttccgggtaagatcgagggttcaaac

SEQ ID NO: 645 - DNA, 27 bp, Glycine max
  source 1..27: mol_type = genomic DNA
tttggctttattcagaaccagaagcct

SEQ ID NO: 646 - DNA, 27 bp, Glycine max
  source 1..27: mol_type = genomic DNA
tttagggtaagcacgaacaggaaagtt

SEQ ID NO: 647 - DNA, 27 bp, Glycine max
  source 1..27: mol_type = genomic DNA
tttaaagagaggtagaatcgtagaagc

SEQ ID NO: 648 - DNA, 27 bp, Glycine max
  source 1..27: mol_type = genomic DNA
tttacaaacataccataaatagttccc

SEQ ID NO: 649 - DNA, 27 bp, Glycine max
  source 1..27: mol_type = genomic DNA
tttagaataacatcacactgcaagaag

SEQ ID NO: 650 - DNA, 27 bp, Glycine max
  source 1..27: mol_type = genomic DNA
tttagaatccaccaacaccaagactgt

SEQ ID NO: 651 - DNA, 27 bp, Glycine max
  source 1..27: mol_type = genomic DNA
tttggacctttctgggtacgtattggg

SEQ ID NO: 652 - DNA, 27 bp, Glycine max
  source 1..27: mol_type = genomic DNA
tttggatataaagtcctctctttcaat

SEQ ID NO: 653 - DNA, 27 bp, Glycine max
  source 1..27: mol_type = genomic DNA
tttcgcatggagtgaaaggcttcttgc

SEQ ID NO: 654 - DNA, 27 bp, Glycine max
  source 1..27: mol_type = genomic DNA
tttagctttaaagagaggtagaatcgt

SEQ ID NO: 655 - DNA, 27 bp, Glycine max
  source 1..27: mol_type = genomic DNA
tttagggagatggatacattcagagag

SEQ ID NO: 656 - DNA, 27 bp, Glycine max
  source 1..27: mol_type = genomic DNA
tttagtgtaacgtggattactgaactt

SEQ ID NO: 657 - DNA, 27 bp, Glycine max
  source 1..27: mol_type = genomic DNA
tttggttagtccagtactcctgttgac

SEQ ID NO: 658 - DNA, 27 bp, Glycine max
  source 1..27: mol_type = genomic DNA
tttagaaatattgaagtcattgcttgc

SEQ ID NO: 659 - DNA, 27 bp, Glycine max
  source 1..27: mol_type = genomic DNA
tttagaccttccaaagggatttgtaag

SEQ ID NO: 660 - DNA, 27 bp, Glycine max
  source 1..27: mol_type = genomic DNA
tttggagggttgatgctaaacatggtg

SEQ ID NO: 661 - DNA, 27 bp, Glycine max
  source 1..27: mol_type = genomic DNA
tttagatgatgagatgacaggttgggt

SEQ ID NO: 662 - DNA, 27 bp, Glycine max
  source 1..27: mol_type = genomic DNA
tttggatgtttagctcaaacgaacttt

SEQ ID NO: 663 - DNA, 27 bp, Glycine max
  source 1..27: mol_type = genomic DNA
tttcgctcaacaccacgacatagttat

SEQ ID NO: 664 - DNA, 27 bp, Glycine max
  source 1..27: mol_type = genomic DNA
tttgggagtgctttgactcgaagacat

SEQ ID NO: 665 - DNA, 27 bp, Glycine max
  source 1..27: mol_type = genomic DNA
tttaggtacatattcggaacttggaaa

SEQ ID NO: 666 - DNA, 27 bp, Glycine max
  source 1..27: mol_type = genomic DNA
tttagtagatggggtggagtaaattgt

SEQ ID NO: 667 - DNA, 27 bp, Glycine max
  source 1..27: mol_type = genomic DNA
tttggtttgtagaccgaagtcaccgaa

SEQ ID NO: 668 - DNA, 27 bp, Glycine max
  source 1..27: mol_type = genomic DNA
tttgtcaacaggagtactggactaacc

SEQ ID NO: 669 - DNA, 27 bp, Glycine max
  source 1..27: mol_type = genomic DNA
tttagtacgattgtacatgtaattaac

SEQ ID NO: 670 - DNA, 27 bp, Glycine max
  source 1..27: mol_type = genomic DNA
tttagattggcccagcgaaggaggcat

SEQ ID NO: 671 - DNA, 27 bp, Glycine max
  source 1..27: mol_type = genomic DNA
tttggcgaatgctatgagtgtgacttc

SEQ ID NO: 672 - DNA, 27 bp, Glycine max
  source 1..27: mol_type = genomic DNA
tttaggtaagaccggtgcacatgaacg

SEQ ID NO: 673 - DNA, 27 bp, Glycine max
  source 1..27: mol_type = genomic DNA
tttgagataattaggatcaagatctcc

SEQ ID NO: 674 - DNA, 27 bp, Glycine max
  source 1..27: mol_type = genomic DNA
tttacgtctatttcaagattaatcaac

SEQ ID NO: 675 - DNA, 27 bp, Glycine max
  source 1..27: mol_type = genomic DNA
tttggcgtatatgctgtttgtgttggt

SEQ ID NO: 676 - DNA, 27 bp, Glycine max
  source 1..27: mol_type = genomic DNA
tttggctaacggtaaagacaagaatgt

SEQ ID NO: 677 - DNA, 27 bp, Glycine max
  source 1..27: mol_type = genomic DNA
tttagctaactccatcatagtgctgtc

SEQ ID NO: 678 - DNA, 27 bp, Glycine max
  source 1..27: mol_type = genomic DNA
tttggatgtttgcgcaccatgcatatg

SEQ ID NO: 679 - DNA, 27 bp, Glycine max
  source 1..27: mol_type = genomic DNA
tttagattgtgttatcaagggtcaact

SEQ ID NO: 680 - DNA, 27 bp, Glycine max
  source 1..27: mol_type = genomic DNA
tttggcatcagacataagcaacaagcc

SEQ ID NO: 681 - DNA, 27 bp, Glycine max
  source 1..27: mol_type = genomic DNA
tttagatatgctgctcacgtgggcagc

SEQ ID NO: 682 - DNA, 27 bp, Glycine max
  source 1..27: mol_type = genomic DNA
tttagcatggtctacgcgcggaacttc

SEQ ID NO: 683 - DNA, 27 bp, Glycine max
  source 1..27: mol_type = genomic DNA
tttagtttaatgatatagaaccactac

SEQ ID NO: 684 - DNA, 27 bp, Glycine max
  source 1..27: mol_type = genomic DNA
tttggaggccaacataggtagctacct

SEQ ID NO: 685 - DNA, 27 bp, Glycine max
  source 1..27: mol_type = genomic DNA
tttaggacctaggaccacggtacttaa

SEQ ID NO: 686 - DNA, 27 bp, Glycine max
  source 1..27: mol_type = genomic DNA
tttggcacacgtgtgctgaatgtgacg

SEQ ID NO: 687 - DNA, 27 bp, Glycine max
  source 1..27: mol_type = genomic DNA
tttggtctcaggacaaggttgctttgg

SEQ ID NO: 688 - DNA, 27 bp, Glycine max
  source 1..27: mol_type = genomic DNA
tttagaatatggtaaatggttcaattc

SEQ ID NO: 689 - DNA, 27 bp, Glycine max
  source 1..27: mol_type = genomic DNA
tttgggtctgccaatgtcatcctgatg

SEQ ID NO: 690 - DNA, 27 bp, Glycine max
  source 1..27: mol_type = genomic DNA
tttggtcgatatagaccgcattatatc

SEQ ID NO: 691 - DNA, 27 bp, Glycine max
  source 1..27: mol_type = genomic DNA
tttggtgcatctgaatgatgagcagag

SEQ ID NO: 692 - DNA, 27 bp, Glycine max
  source 1..27: mol_type = genomic DNA
tttggcaggtttactgtgttctcactg

SEQ ID NO: 693 - DNA, 27 bp, Glycine max
  source 1..27: mol_type = genomic DNA
tttaggtgaagtttataagccttgaat

SEQ ID NO: 694 - DNA, 27 bp, Glycine max
  source 1..27: mol_type = genomic DNA
tttagtgggacgggtcaaacccattcc

SEQ ID NO: 695 - DNA, 27 bp, Glycine max
  source 1..27: mol_type = genomic DNA
tttagtccgaatggtgaacgggactct

SEQ ID NO: 696 - DNA, 27 bp, Glycine max
  source 1..27: mol_type = genomic DNA
tttaggggattgcagggtgtctaccgg

SEQ ID NO: 697 - DNA, 27 bp, Glycine max
  source 1..27: mol_type = genomic DNA
tttagtttcaccagcctctcgttattc

SEQ ID NO: 698 - DNA, 27 bp, Glycine max
  source 1..27: mol_type = genomic DNA
tttagaaacaacggtaaagcatattaa

SEQ ID NO: 699 - DNA, 27 bp, Glycine max
  source 1..27: mol_type = genomic DNA
tttaacagaatatttcagccggtagac

SEQ ID NO: 700 - DNA, 27 bp, Glycine max
  source 1..27: mol_type = genomic DNA
tttagtacatctacaacaaagcaaagg

SEQ ID NO: 701 - DNA, 27 bp, Glycine max
  source 1..27: mol_type = genomic DNA
tttggtcggagcctgttaccggaacag

SEQ ID NO: 702 - DNA, 27 bp, Glycine max
  source 1..27: mol_type = genomic DNA
tttagtcataagtaacaatggtagcat

SEQ ID NO: 703 - DNA, 27 bp, Glycine max
  source 1..27: mol_type = genomic DNA
tttgatcatacaagcgtttataaaccc

SEQ ID NO: 704 - DNA, 27 bp, Glycine max
  source 1..27: mol_type = genomic DNA
tttggttaccatggaagccatgtatca

SEQ ID NO: 705 - DNA, 27 bp, Glycine max
  source 1..27: mol_type = genomic DNA
tttcgctcgctatatatatgtgtcaca

SEQ ID NO: 706 - DNA, 27 bp, Glycine max
  source 1..27: mol_type = genomic DNA
tttgcaaacatcacatggccggataag

SEQ ID NO: 707 - DNA, 27 bp, Glycine max
  source 1..27: mol_type = genomic DNA
tttacatgctgtcatcatctgttcact

SEQ ID NO: 708 - DNA, 27 bp, Glycine max
  source 1..27: mol_type = genomic DNA
tttaccttgatttggattaacccgcca

SEQ ID NO: 709 - DNA, 27 bp, Glycine max
  source 1..27: mol_type = genomic DNA
tttacttcctgaggaaatttggtttca

SEQ ID NO: 710 - DNA, 20 bp, unidentified
  source 1..20: mol_type = unassigned DNA; note = Lachnospiraceae bacterium ND2006
aatttctactaagtgtagat

SEQ ID NO: 711 - DNA, 21 bp, unidentified
  source 1..21: mol_type = unassigned DNA; note = Lachnospiraceae bacterium ND2006
taatttctactaagtgtagat

(19) United States
(12) Patent Application Publication
(10) Pub. No.: US 2026/0218225 A1
(43) Pub. Date: Jul. 30, 2026

(54) COMPOSITIONS AND METHODS FOR SITE-DIRECTED INTEGRATION

(71) Applicant: Monsanto Technology LLC, St. Louis, MO (US)

(72) Inventors: Robert Gaeta, Eureka, MO (US); Zane Goodwin, St. Louis, MO (US); Linda Rymarquis, High Ridge, MO (US)

(21) Appl. No.: 19/134,560

(22) PCT Filed: Dec. 7, 2023
(86) PCT No.: PCT/US2023/082952

§ 371 Date: May 30, 2025

Related U.S. Application Data
(60) Provisional application No. 63/387,871, filed on Dec. 16, 2022.

(51) Int. Cl. C12N 15/82; C12N 9/22; C12N 15/11

(52) U.S. Cl. CPC C12N 15/8274; C12N 9/226; C12N 15/11; C12N 15/8213; C12N 2310/20

(57) ABSTRACT

Target sites for site-directed integration of a DNA sequence of interest near the soybean HT4 event (soybean event GM_CSM63714) insertion site are provided. Soy plants, plant seeds, plant parts, plant cells and progeny plants comprising the target sites are also provided. Methods of generating recombinant soy plant cells comprising the target sites and a DNA sequence of interest are further provided.

### CROSS-REFERENCE TO RELATED APPLICATIONS

This application claims the benefit of U.S. Provisional Application No. 63/387,871, filed Dec. 16, 2022, which is incorporated herein by reference in its entirety.

### INCORPORATION OF SEQUENCE LISTING

The sequence listing that is contained in the file named “MONS549WO_ST26.xml,” which is 615 kilobytes as measured in Microsoft Windows operating system and was created on Oct. 19, 2023, is filed electronically herewith and incorporated herein by reference.

### FIELD OF THE INVENTION

The present disclosure relates to the field of agricultural biotechnology, and more specifically to methods and compositions for selecting target sites for site-specific genome modification in plant genomes.

### BACKGROUND OF THE INVENTION

Site-specific genome modification in plant genomes provides a means to develop plants with specific traits and to facilitate plant breeding programs. For the development of new agronomic traits, site-specific genome modification enzymes are used for site-specific genome editing, and for site-specific targeted integration of a DNA of interest. Site-specific transgene integration in a plant genome provides significant improvement over random integration of a transgene in the development of new traits.

There is a need to identify suitable target sites for site-specific genome modification in a plant genome.

### SUMMARY

The present disclosure describes genomic regions that are suitable as target sites of site-specific genome modification enzymes, and the target sites so identified. Site-specific integration of DNA of interest will reduce development costs and increase optimal agronomic trait development in the site-specific modified plant genome.

The present disclosure provides a recombinant DNA molecule comprising a DNA sequence having at least 85% sequence identity, at least 90% sequence identity, or at least 95% sequence identity to a nucleic acid molecule selected from the group consisting of nucleotides 5-27 of SEQ ID NOs:1-709. In certain embodiments, the recombinant DNA molecule comprises a nucleic acid molecule selected from the group consisting of nucleotides 5-27 of SEQ ID NOs:1-709. In some embodiments, the DNA sequence is operably linked to a heterologous promoter sequence. In other embodiments the recombinant DNA molecule further comprises SEQ ID NO:710 or SEQ ID NO:711.

The present disclosure also provides a recombinant RNA molecule comprising an RNA sequence that is at least 85% complementary, at least 90% complementary, or at least 95% complementary to a nucleic acid molecule selected from the group consisting of nucleotides 5-27 of SEQ ID NOs:1-709. In particular embodiments, the RNA sequence is 100% complementary to a nucleic acid molecule selected from the group consisting of nucleotides 5-27 of SEQ ID NOs:1-709.

The present disclosure further provides a soy plant, plant seed, plant part, plant cell or progeny plant comprising a recombinant nucleic acid molecule, said recombinant nucleic acid molecule comprising a target soy genomic nucleic acid sequence having at least 85% sequence identity, at least 90% sequence identity, or at least 95% sequence identity to a nucleic acid molecule selected from the group consisting of SEQ ID NOs:1-709; and a DNA sequence of interest, wherein the DNA sequence of interest is inserted into said target soy genomic nucleic acid sequence. In certain embodiments, the soy plant, seed, plant part, plant cell or progeny plant comprises a recombinant nucleic acid molecule, said recombinant nucleic acid molecule comprising a target soy genomic nucleic acid sequence having a sequence selected from the group consisting of SEQ ID NOs:1-709. In some embodiments, the DNA sequence of interest comprises a gene of agronomic interest. In other embodiments the gene of agronomic interest confers herbicide tolerance in plants. In certain embodiments, the target soy genomic nucleic acid molecule is at least 1 kb, 10 kb, 50 kb, 100 kb, 250 kb, 500 kb or 1000 kb from a soy event Gm_CSM63714 insertion site. In further embodiments, the target soy genomic nucleic acid molecule is at least 1 cM from a soy event Gm_CSM63714 insertion site. In yet other embodiments, the target soy genomic nucleic acid sequence maps to within 1 cM, 2 cM, 3 cM, 4 cM or 5 cM of the soy event Gm_CSM63714 insertion site.

The present disclosure additionally provides a method of generating a recombinant soy plant cell comprising the following steps: a) obtaining a soy plant, seed, or cell, wherein said plant, seed, or cell comprises a target soy genomic nucleic acid molecule having at least 85% sequence identity, at least 90% sequence identity, or at least 95% sequence identity to a nucleic acid molecule selected from the group consisting of SEQ ID NOs:1-709; b) introducing into the soy plant, seed, or cell a site-specific nuclease that can specifically bind to and cleave the target soy genomic nucleic acid molecule; c) introducing a DNA sequence of interest into the soy plant, seed, or cell; d) inserting the DNA sequence of interest into the target soy genomic nucleic acid molecule; and e) selecting recombinant soy plants, seeds or cells comprising the DNA sequence of interest inserted in the target soy genomic nucleic acid molecule. In various embodiments, site-specific nuclease is selected from the group consisting of an RNA-guided nuclease, a zinc finger nuclease and a TALEN. In certain embodiments, the RNA-guided nuclease is Cas12a. In additional embodiments, the method further comprising introducing into the soy plant, seed, or cell a guide polynucleotide comprising a nucleic acid sequence that is substantially complementary to the target soy genomic nucleic acid, wherein the guide polynucleotide and the RNA-guided nuclease form a complex that can bind to and cleave the soy genomic nucleic acid molecule. In some embodiments, the guide polynucleotide comprises a nucleotide sequence having at least at least 85% sequence identity, at least 90% sequence identity, or at least 95% sequence identity to a nucleic acid molecule selected from the group consisting of nucleotides 5-27 of SEQ ID NOs:1-709. In other embodiments, the guide polynucleotide further comprises SEQ ID NO:710 or SEQ ID NO:711. In yet other embodiments, the target soy genomic nucleic acid molecule is at least 1 kb, 10 kb, 50 kb, 100 kb, 250 kb, 500 kb or 1000 kb from a soy event Gm_CSM63714 insertion site. In further embodiments, the target soy genomic nucleic acid molecule is at least 1 cM from a soy event Gm_CSM63714 insertion site. In still other embodiments the target soy genomic nucleic acid sequence maps to within 1 cM, 2 cM, 3 cM, 4 cM or 5 cM of the soy event Gm_CSM63714 insertion site, either proximal or distal relative to the centromere of the chromosome in which the Gm_CSM63714 insertion site is located.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a schematic illustration of an enlarged region of chromosome 13 on the soybean (Glycine max) genome showing the location of the soybean event GM_CSM63714 (designated as “HT-4”) integration site and the region that maps to within 1 to 5 cM of the GM_CSM63714 event integration site.

FIG. 2 shows a general work-flow diagram illustrating one embodiment of steps in a method of site selection for targeted integration.

### BRIEF DESCRIPTION OF THE SEQUENCES

SEQ ID NOs:1-709 are target sites that map to within 1 to 5 cM of the GM_CSM63714 event integration site in the soybean genome.

SEQ ID NOs:710 and 711 are crRNA scaffold sequences utilized by LbCas12a gRNAs.

### DETAILED DESCRIPTION

Site-specific genome modification allows for the introduction of DNA sequences of interest into a plant cell, and is useful for the development of plants expressing traits of agronomic interest. Site-specific transgene integration in a plant genome provides significant improvement over random integration of a transgene in the development of new traits.

However, in order to direct site-specific genome modification, target sequences within a plant genome must be identified to optimize the beneficial effect of the modification to be made. The present disclose therefore provides novel target sequences for site-specific modification of plant genomes.

### I. Genome Editing

The present disclosure provides, in certain embodiments, target sites for site-directed integration or site-specific integration of a DNA sequence of interest into a specific location in the genome of a plant, and plants, plant parts, plant cells, and seeds produced through genome modification using site-directed or site-specific integration, or in certain embodiments genome editing. As used herein, the term “target site,” “genomic target site,” “target genomic nucleic acid,” or “target soy genomic nucleic acid,” refers to a polynucleotide sequence that is sufficiently unique in the soy genome to allow targeted genome modification by a site-specific nuclease. In one aspect, the sequence of the target site is changed from the wild-type sequence, namely the target site is edited. In another aspect, the target site is the site of insertion of a DNA sequence of interest.

In certain embodiments, the target site can comprise one or more of the criteria selected from the group consisting of: (i) the target site selected is unique in the soy A3555 genome; (ii) the target site is not located in a gene or coding sequence; (iii) the target site selected is more than 1 kb from a gene, (iv) the target site selected is more than 1 kb from a repressive chromatin mark (e.g., H3K27me3 peak), (v) the target site selected is more than 200 nucleotides (nt) from a small RNA hotspot, (vi) the target site selected is more than 1 kb from a long repeat region, (vii) the target site selected has low DNA methylation (less than or equal to 10% of genome-wide population average) (viii) the target site selected has low redundancy score (less than or equal to 30%), (ix) the target site selected does not have potential off-targets, wherein the off-targets are defined as sites matching the gRNA hybridization site within the target site with three or fewer mismatches and containing a TTTN PAM or NTTN PAM, where N is A, T, G or C; including any combination of two or more thereof.

The target site comprises a sequence that is recognized by a site-specific nuclease. In some embodiments, the target site comprises a sequence that is recognized by a site-specific nuclease resulting in precise or targeted cleavage within the target site. For example, the site-specific nuclease can be selected from the group consisting of: an RNA-guided nuclease, a zinc Finger nuclease, and a TALEN. In some embodiments, the target site comprises a PAM (Protospacer Adjacent Motif) sequence that is recognized by an RNA-guided nuclease (e.g., a CRISPR nuclease system). For example, the target site can comprise a PAM motif that is recognized by a Cas12a/Cpf1 CRISPR nuclease system. The target site can further comprise a sequence that is recognized by and hybridizes to a CRISPR guide RNA. In some embodiments, the target site comprises a sequence that is recognized by and hybridizes to a Cas12a/Cpf1 CRISPR guide RNA.

A DNA sequence of interest can be inserted at a target site using a site-specific nuclease. As used herein, the term “DNA sequence of interest” or “donor sequence” or “donor DNA” refers to a nucleic acid/DNA sequence that has been selected for targeted insertion into a soybean genomic sequence. In one aspect, the soybean genomic sequence is a genomic target site described herein. A DNA sequence on interest can be of any length, for example between 2 and 50,000 nucleotides in length (or any integer value therebetween). In some embodiments, the DNA sequence is between about 1,000 and 5,000 nucleotides in length (or any integer value therebetween). In some embodiments, the DNA sequence is between about 5,000 and 10,000 nucleotides in length (or any integer value therebetween). In some embodiments, the DNA sequence is between about 10,000 and 15,000 nucleotides in length (or any integer value therebetween). In some embodiments, the DNA sequence is between about 15,000 and 20,000 nucleotides in length (or any integer value therebetween). In some embodiments, the DNA sequence is between about 20,000 and 25,000 nucleotides in length (or any integer value therebetween). In some embodiments, the DNA sequence is between about 25,000 and 30,000 nucleotides in length (or any integer value therebetween). In some embodiments, the DNA sequence is between about 30,000 and 35,000 nucleotides in length (or any integer value therebetween). In some embodiments, the DNA sequence is between about 35,000 and 40,000 nucleotides in length (or any integer value therebetween). In some embodiments, the DNA sequence is between about 40,000 and 45,000 nucleotides in length (or any integer value therebetween). In some embodiments, the DNA sequence is between about 45,000 and 50,000 nucleotides in length (or any integer value therebetween).

A DNA sequence may comprise one or more gene expression cassettes that further comprise actively transcribed and/or translated gene sequences. For example, the DNA sequence of interest can comprise a gene expression cassette comprising a sequence selected from: an herbicide tolerance gene, an insecticidal resistance gene, a nitrogen use efficiency gene, a water use efficiency gene, a nutritional quality gene, a DNA binding gene, a selectable marker gene, a target site for a site-specific nuclease, and any combination thereof. Alternatively, the DNA sequence of interest may comprise a polynucleotide sequence which does not comprise a functional gene expression cassette or an entire gene (e.g., may comprise regulatory sequences such as a promoters, enhancers, etc.), or may not contain any identifiable gene expression elements or any actively transcribed gene sequence. In some embodiments, the DNA of interest will have at least one homology arm DNA sequence. The term “homology arm DNA sequence” refers to a polynucleotide sequence that has at least 80%, at least 85%, at least 90%, at least 91%, at least 92%, at least 93%, at least 94%, at least 95%, at least 96%, at least 97%, at least 98%, at least 99% or 100% sequence identity to a target sequence in a plant or plant cell. Further, the DNA sequence can be linear or circular, and can be single-stranded or double-stranded. It can be delivered to the cell as naked nucleic acid, as a complex with one or more delivery agents (e.g., liposomes, poloxamers, T-strand encapsulated with proteins, etc.) or contained in a bacterial or viral delivery vehicle, such as, for example, Agrobacterium tumefaciens or a Gemini Virus, or a nanovirus, respectively.

Once a specific target site is identified, a site-specific nuclease targeting the selected target site can be designed and introduced into the plant, seed, or plant cell. For example, a CRISPR-associated nuclease (e.g., Cas12a/Cpf1) and at least one RNA guide molecule that can hybridize to the target site can be designed and cloned into a plant expression vector and delivered to the plant, seed, or plant cell. If the genome modification is designed to induce a double-strand break (DSB) (i.e., induce a cleavage) with non-homologous end joining (NHEJ) repair for introduction of insertions and deletions (indels), then just the engineered CRISPR nuclease and at least one RNA guide molecule are delivered to the plant, seed or cell. If a DNA sequence of interest is to be incorporated at the target site, then the engineered CRISPR nuclease, at least one RNA guide molecule, and the DNA of interest are co-delivered to the plant, seed, or cell. The DNA of interest may integrate into the target site by NHEJ (Non-homologous End Joining) or by homology-dependent repair (HR). In the latter case, the DNA of interest will have at least one homology arm DNA sequence. An alternative to delivery of the engineered CRISPR nuclease as a DNA expression construct is the delivery of a Ribonucleo-protein (RNP) complex of the CRISPR-associated nuclease protein in complex with the guide RNA.

Following delivery of the site-specific nuclease to a plant cell, the cells or plants regenerated from the cells are sampled to confirm the presence of the intended site-specific genome modification including insertion of the DNA sequence of interest at or proximal to the target site. Methods of detecting the genome modification are known to one skilled in the art, and include PCR, TaqMan® PCR, droplet digital PCR (ddPCR™, Bio-Rad Laboratories, Hercules, Calif.), sequencing, Sanger sequencing, ABI 3730 DNA fragment analysis (Applied Biosystems, Grand Island, N.Y.), Southern analysis, Northern analysis, phenotypic analysis, or any other technique known to one in the art to detect genome modification.

As further described in Example 1 hereinbelow, 709 target sites within 5 centimorgan (cM) upstream and downstream of the soybean GM_CSM63714 (also having the designation HT-4) were identified. The sequences of these target sites are provided herein as SEQ ID NOs:1-709. Guide RNA spacer sequences corresponding to each of the target site sequences are provided as nucleotides 5-27 of SEQ ID NOs:1-709.

Soybean plants, plant seeds, plant parts, plant cells, and progeny plants are provided. The plant, seed, plant part, plant cell, or progeny plant comprises a recombinant nucleic acid molecule. The recombinant nucleic acid molecule comprises a target soy genomic nucleic acid sequence having at least 85% sequence identity, at least 90% sequence identity, at least 95% sequence identity, or up to 100% sequence identity to a nucleic acid molecule selected from the group consisting of SEQ ID NOs:1-709. The recombinant nucleic acid molecule further comprises a DNA sequence of interest. The DNA sequence of interest is inserted into said target soy genomic nucleic acid sequence. The DNA sequence of interest can comprise a gene of agronomic interest. For example, the gene of agronomic interest can confer herbicide tolerance in plants.

A method of generating a recombinant soybean plant cell is provided, the method comprising the following steps: (a) obtaining a soybean plant, seed, or cell, wherein said plant, seed, or cell comprises a target soybean genomic nucleic acid molecule having at least 85% sequence identity, at least 90% sequence identity, at least 95% sequence identity, or up to 100% sequence identity to a nucleic acid molecule selected from the group consisting of SEQ ID NOs:1-709; (b) introducing into the soybean plant, seed, or cell a site-specific nuclease that can specifically bind to and cleave the target soybean genomic nucleic acid molecule; (c) introducing a DNA sequence of interest into the soybean plant, seed, or cell; (d) inserting the DNA sequence of interest into the target soybean genomic nucleic acid molecule; and (e) selecting recombinant soybean plants, seeds or cells comprising the DNA sequence of interest inserted in the target soybean genomic nucleic acid molecule. The site-specific nuclease can be selected from the group consisting of an RNA-guided nuclease, a zinc finger nuclease and a TALEN. For example, the RNA-guided nuclease can be Cas12a. The method can further comprise introducing into the soybean plant, seed, or cell a guide polynucleotide comprising a nucleic acid sequence that is substantially complementary to the target soy genomic nucleic acid, wherein the guide polynucleotide and the RNA-guided nuclease form a complex that can bind to and cleave the soybean genomic nucleic acid molecule. The guide polynucleotide can comprise a nucleotide sequence having at least at least 85% sequence identity, at least 90% sequence identity, at least 95% sequence identity, or up to 100% sequence identity to a nucleic acid molecule selected from the group consisting of nucleotides 5-27 of SEQ ID NOs:1-709.

A recombinant DNA molecule is provided. The recombinant DNA molecule comprises a DNA sequence having at least 85% sequence identity, at least 90% sequence identity, at least 95% sequence identity, or up to 100% sequence identity to a nucleic acid molecule selected from the group consisting of nucleotides 5-27 of SEQ ID NOs:1-709. The DNA sequence can be operably linked to a heterologous promoter sequence. The recombinant DNA molecule can further comprise SEQ ID NOs:710 or 711.

A recombinant RNA molecule is provided. The recombinant RNA molecule comprises an RNA sequence that is at least 85% complementary, at least 90% complementary, at least 95% complementary, or up to 100% complementary to a nucleic acid molecule selected from the group consisting of nucleotides 5-27 of SEQ ID NOs:1-709.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as an RNA-guided endonuclease (e.g., the CRISPR/Cas12a system), a TALE (transcription activator-like effector)-endonuclease (TALEN), a zinc-finger nuclease (ZFN), a meganuclease, a recombinase, or a transposase. As used herein, “editing” or “genome editing” may encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted insertion, whereas “edits” or “genomic edits” refers to two or more targeted insertion(s), with each “edit” being introduced via a targeted genome editing technique.

According to some embodiments, a site-specific nuclease may be co-delivered with a donor template molecule to serve as a template for making a desired insertion into the genome at the desired target site through repair of the double strand break (DSB) or nick created by the site-specific nuclease. In some embodiments, said double-strand break(s) or said nick(s) leads to the formation of one or more insertion or deletion mutations (INDELs) in the target site DNA sequences. In some embodiments, said double-strand break(s) or said nick(s) leads to the insertion of donor template molecule into the genome in the target site DNA sequences. According to some embodiments, a site-specific nuclease may be co-delivered with a DNA molecule comprising a selectable or screenable marker gene.

A site-specific nuclease provided herein may be selected from the group consisting of an RNA-guided endonuclease, a zinc-finger nuclease (ZFN), a TALE-endonuclease (TALEN), a meganuclease, a recombinase, a transposase, or any combination thereof. See, e.g., Khandagale et al. (Plant Biotechnol Rep 10:327-343, 2016); and Gaj et al. (Trends Biotechnol. 31(7):397-405, 2013.

A site-specific nuclease may be an RNA-guided nuclease. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions of any thereof, as well as Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 enzyme. The RNA-guided nuclease may be delivered as a protein with or without a guide RNA, or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the plant via base-pairing or hybridization to cause a DSB or nick at or near the target site. The guide RNA may be transformed or introduced into a plant cell or tissue as a gRNA molecule, or as a recombinant DNA molecule, construct or vector comprising a transcribable DNA sequence encoding the guide RNA operably linked to a promoter. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR-associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). A “single-chain guide RNA” (or “sgRNA”) is an RNA molecule comprising a crRNA covalently linked a tracrRNA by a linker sequence, which may be expressed as a single RNA transcript or molecule. The guide RNA comprises a guide or targeting sequence (also referred to herein as a “spacer sequence”) that is identical or complementary to a target site within the plant genome. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 80%, at least 85%, at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

Zinc finger nucleases (ZFN) are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site but may typically be composed of 3-4 (or more) zinc-fingers. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., U.S. Patent Application Publication Nos. 2005/0064474, 2009/0117617, and 2012/0142062. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

TALENs are artificial restriction enzymes generated by fusing the TALE DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al., PLoS One 8:e82539, 2013). MutH is capable of introducing strand-specific nicks in DNA (see Gabsalilow et al., Nucleic Acids Research. 41:e83, 2013). TevI introduces double-stranded breaks in DNA at targeted sites (see Beurdeley et al., Nature Communications 4:1762, 2013).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al. (Nucleic Acids Research 40:W117-122, 2012); Cermak et al. (Nucleic Acids Research 39:e82, 2011); and tale-nt.cac.cornell.edu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

As used herein, with respective to a given sequence, a “complement”, a “complementary sequence” and a “reverse complement” are used interchangeably. All three terms refer to the inversely complementary sequence of a nucleotide sequence, i.e., to a sequence complementary to a given sequence in reverse order of the nucleotides.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

Depending on the CRISPR nuclease-gRNA system, a protospacer-adjacent motif (PAM) may be present in the genome immediately adjacent and upstream to the 5′ end or immediately adjacent and downstream to the 3′ end of the guide RNA hybridization sequence (also known as protospacer), i.e., immediately downstream (3′) to the sense (+) strand of the protospacer as known in the art. See, e.g., Wu et al. (Quant Biol. 2(2):59-70, 2014). The genomic target site comprises the reverse complement of the PAM+protospacer or protospacer+PAM. Length of the protospacer and sequence of the PAM is determined by the species of CRISPR enzyme used. For example, for Cas12a the PAM sequence 5′-TTTV-3′ is present immediately adjacent and 5′ to the guide RNA protospacer. For Cas9, the PAM sequence 5′-NGG-3′ (where N is ‘A’, ‘T’, ‘C’ or ‘G’) is present immediately downstream and 3′ to the guide RNA protospacer.

In some embodiments, a site-specific nuclease is a recombinase. Non-limiting examples of recombinases that may be used include a serine recombinase attached to a DNA recognition motif, a tyrosine recombinase attached to a DNA recognition motif, or any recombinase enzyme known in the art attached to a DNA recognition motif. In certain embodiments, the site-specific nuclease is a recombinase or transposase, which may be a DNA transposase or recombinase attached or fused to a DNA binding domain. Non-limiting examples of recombinases include a tyrosine recombinase selected from the group consisting of a Cre recombinase, a Gin recombinase, a Flp recombinase, and a Tnp1 recombinase attached to a DNA recognition motif provided herein. In one aspect of the present disclosure, a Cre recombinase or a Gin recombinase provided herein is tethered to a zinc-finger DNA-binding domain, a TALE DNA-binding domain, or a Cas12a nuclease. In another aspect, a serine recombinase selected from the group consisting of a PhiC31 integrase, an R4 integrase, and a TP-901 integrase may be attached to a DNA recognition motif provided herein. In yet another aspect, a DNA transposase selected from the group consisting of a TALE-piggyBac and TALE-Mutator may be attached to a DNA binding domain provided herein.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such a genomic locus. The DSB or nick created by the non-RNA-guided site specific nuclease may lead to insertion of a sequence at the site of the DSB or nick through cellular repair mechanisms. Such cellular repair mechanism may be guided by a donor template molecule.

As used herein, a “donor molecule,” “donor template,” or “donor template molecule,” (collectively a “donor template”) which may be a recombinant polynucleotide, DNA, or RNA donor template or sequence, is defined as a nucleic acid molecule having a homologous nucleic acid template or sequence (e.g., homology sequence) and/or an insertion sequence for site-directed, targeted insertion or recombination into the genome of a plant cell via repair of a nick or DSB in the genome of a plant cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct into a target site within the genome of a plant. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes or transcribable DNA sequences.

Without being limiting, a gene/transgene or transcribable DNA sequence of a donor template may include, for example, an insecticidal resistance gene, an herbicide tolerance gene, a nitrogen use efficiency gene, a water use efficiency gene, a yield enhancing gene, a nutritional quality gene, a DNA binding gene, a selectable marker gene, an RNAi or suppression construct, a site-specific genome modification enzyme gene, a single guide RNA of a CRISPR/Cas12a system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter operably linked to a coding sequence, gene, or transcribable DNA sequence, such as a constitutive promoter, a tissue-specific or tissue-preferred promoter, a developmental stage promoter, or an inducible promoter. A donor template may comprise a leader, enhancer, promoter, transcriptional start site, 5′-UTR, one or more exon(s), one or more intron(s), transcriptional termination site, region or sequence, 3′-UTR, and/or polyadenylation signal, which may each be operably linked to a coding sequence, gene (or transgene) or transcribable DNA sequence encoding a non-coding RNA, a guide RNA, an mRNA and/or protein. A donor template may be a single-stranded or double-stranded DNA or RNA molecule or plasmid.

An “insertion sequence” of a donor template or donor sequence is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template. Each homology arm may be at least 70%, at least 75%, at least 80%, at least 85%, at least 90%, at least 95%, at least 96%, at least 97%, at least 98/%, at least 99%, or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant.

Any method known in the art for site-directed integration may be used with the present disclosure. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

### II. Constructs for Genome Editing

Recombinant DNA constructs and vectors are provided comprising a polynucleotide sequence encoding a site-specific nuclease, such as an RNA-guided endonuclease, a zinc-finger nuclease (ZFN), a meganuclease, a TALE-endonuclease (TALEN), a recombinase, or a transposase, wherein the coding sequence is operably linked to a plant expressible promoter. For RNA-guided endonucleases, recombinant DNA constructs and vectors are further provided comprising a polynucleotide sequence encoding a guide RNA, wherein the guide RNA comprises a guide sequence of sufficient length having a percent identity or complementarity to a target site within the genome of a plant, such as at or near a targeted soybean event GM_CSM63714 insertion site. A polynucleotide sequence of a recombinant DNA construct and vector that encodes a site-specific nuclease or a guide RNA may be operably linked to a plant expressible promoter, such as an inducible promoter, a constitutive promoter, a tissue-specific promoter, etc.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, an “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant or edited allele for a gene may have a reduced or eliminated activity or expression level for the gene relative to the wild-type allele. For diploid organisms such as soybean, a first allele can occur on one chromosome, and a second allele can occur at the same locus on a second homologous chromosome. If one allele at a locus on one chromosome of a plant is a mutant or edited allele and the other corresponding allele on the homologous chromosome of the plant is wild-type, then the plant is described as being heterozygous for the mutant or edited allele. However, if both alleles at a locus are mutant or edited alleles, then the plant is described as being homozygous for the mutant or edited alleles. A plant homozygous for mutant or edited alleles at a locus may comprise the same mutant or edited allele or different mutant or edited alleles if heteroallelic or biallelic.

As used herein, a “wild-type gene” or “wild-type allele” refers to a gene or allele having a sequence or genotype that is most common in a particular plant species, or another sequence or genotype having only natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele relative to the most common sequence or genotype. As used herein, a “native copy” of a gene refers to a gene that originates from within a given organism, cell, tissue, genome, or chromosome that was not previously modified by human action. Similarly, a “native protein” refers to a protein encoded by a native gene.

In general, the term “variant” refers to molecules with some differences, generated synthetically or naturally, in their nucleotide or amino acid sequences as compared to a reference (native) polynucleotides or polypeptides, respectively. These differences include substitutions, insertions, deletions or any desired combinations of such changes in a native polynucleotide or amino acid sequence.

As used herein, the term “expression” refers to the biosynthesis of a gene product, and typically the transcription and/or translation of a nucleotide sequence, such as an endogenous gene, a heterologous gene, a transgene or an RNA and/or protein coding sequence, in a cell, tissue, organ, or organism, such as a plant, plant part or plant cell, tissue or organ.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., can also refer to a polynucleotide or protein molecule or sequence that has been genetically engineered and/or constructed outside of a cell. For example, a recombinant DNA molecule can comprise any engineered or man-made plasmid, vector, etc., and can include a linear or circular DNA molecule. Such plasmids, vectors, etc., can contain various maintenance elements including a prokaryotic origin of replication and selectable marker, as well as one or more transgenes or expression cassettes perhaps in addition to a plant selectable marker gene, etc. The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

Reference in this application to an “isolated DNA molecule” or an “isolated polynucleotide”, or an equivalent term or phrase, is intended to mean that the DNA molecule or polynucleotide is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding a protein or any naturally occurring variant of that protein would be an isolated nucleotide sequence so long as the nucleotide sequence was not within the DNA of the organism in which the sequence encoding the protein is naturally found. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, or present in an extrachromosomal vector, would be considered to be an isolated nucleotide sequence whether it is present within the plasmid or similar structure used to transform the cells, within the genome of the plant, or present in detectable amounts in tissues, progeny, biological samples or commodity products derived from the plant.

As commonly understood in the art, the term “promoter” can generally refer to a DNA sequence that contains an RNA polymerase binding site, transcription start site, and/or TATA box and assists or promotes the transcription and expression of an associated transcribable polynucleotide sequence and/or gene (or transgene). The promoter sequence is located upstream or 5′ to a transcribable sequence and is involved in recognition and binding of RNA polymerase I, II, or III and other proteins (trans-acting transcription factors) to initiate transcription. A promoter can be synthetically produced, varied or derived from a known or naturally occurring promoter sequence or other promoter sequence. A promoter can also include a chimeric promoter comprising a combination of two or more heterologous sequences. A promoter of the present disclosure can thus include variants or fragments of promoter sequences that are similar in composition, but not identical to, other promoter sequence(s) known or provided herein. In some embodiments, the promoter is recognized by an RNA Polymerase III enzyme which transcribes small nuclear RNAs (snRNAs). For example, native polymerase III promoters, such as the U6 snRNA promoters can be used to drive expression of gRNAs (see U.S. Pat. No. 11,186,843). Other examples of RNA polymerase III promoters include U3, U2, U5 and 7SL promoters. A promoter provided herein, or variant or fragment thereof, may also comprise a “minimal promoter” which provides a basal level of transcription and is comprised of a TATA box or equivalent DNA sequence for recognition and binding of the RNA polymerase II complex for initiation of transcription. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc.

As used herein, a “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature without human introduction—e.g., the promoter or regulatory sequence has a different origin relative to the associated polynucleotide sequence and/or the promoter or regulatory sequence is not naturally occurring in a plant species to be transformed with the promoter or regulatory sequence.

As used herein, an “endogenous gene” or an “endogenous locus” refers to a gene or locus at its natural and original chromosomal location.

As used herein, in the context of a protein-coding gene, an “exon” refers to a segment of a DNA or RNA molecule containing information coding for a protein or polypeptide sequence.

As used herein, an “intron” of a gene refers to a segment of a DNA or RNA molecule, which does not contain information coding for a protein or polypeptide, and which is first transcribed into an RNA sequence but then spliced out from a mature RNA molecule.

As used herein, an “untranslated region (UTR)” of a gene refers to a segment of an RNA molecule or sequence (e.g., a mRNA molecule) expressed from a gene (or transgene), but excluding the exon and intron sequences of the RNA molecule. An “untranslated region (UTR)” also refers a DNA segment or sequence encoding such a UTR segment of an RNA molecule. An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream (5′) or downstream (3′) of the exon and intron sequences, respectively).

As used herein, a “transcription termination sequence” refers to a nucleic acid sequence containing a signal that triggers the release of a newly synthesized transcript RNA molecule from an RNA polymerase complex and marks the end of transcription of a gene or locus.

As used herein, a “homolog” or “homologues” means a protein in a group of proteins that perform the same biological function, for example, proteins that belong to the same protein family and that provide a common enhanced trait in modified plants of this disclosure. Homologs are expressed by homologous genes. With reference to homologous genes, homologs include orthologs, for example, genes expressed in different species that evolved from common ancestral genes by speciation and encode proteins retain the same function, but do not include paralogs, i.e., genes that are related by duplication but have evolved to encode proteins with different functions. Homologous genes include naturally occurring alleles and artificially-created variants.

Homologs are inferred from sequence similarity, by comparison of protein sequences, for example, manually or by use of a computer-based tool. For optimal alignment of sequences to calculate their percent identity, various pair-wise or multiple sequence alignment algorithms and programs are known in the art, such as ClustalW or Basic Local Alignment Search Tool® (BLAST), etc., that can be used to compare the sequence identity or similarity between two or more nucleotide or protein sequences. BLAST, can also be used, for example to search query protein sequences of a base organism against a database of protein sequences of various organisms, to find similar sequences. The generated summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process orthologs are further differentiated from paralogs among all the homologs, which allows for the inference of functional equivalence of genes.

The terms “percent identity,” “% identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.” Sequences having a percent identity to a base sequence may exhibit the activity of the base sequence.

Degeneracy of the genetic code provides the possibility to substitute at least one base of the protein encoding sequence of a gene with a different base without causing the amino acid sequence of the polypeptide produced from the gene to be changed. When optimally aligned, homolog proteins, or their corresponding nucleotide sequences, have typically at least about 70%, at least about 75%, at least about 80%, at least about 85%, at least about 90%, at least about 92%, at least about 93%, at least about 94%, at least about 95%, at least about 96%, at least about 97%, at least about 98%, at least about 99%, or even at least about 99.5% identity over the full length of a protein or its corresponding nucleotide sequence.

Homologs of the proteins described herein are also identifiable by the presence of a conserved functional domain(s). The term “domain” refers to a set of amino acids conserved at specific positions along an alignment of sequences of evolutionarily related proteins. While amino acids at other positions can vary between homologs, amino acids that are highly conserved at specific positions indicate amino acids that are essential in the structure, stability, or activity of a protein. Identified by their high degree of conservation in aligned sequences of a family of protein homologs, they can be used as identifiers to determine if any polypeptide in question belongs to a previously identified polypeptide family (in this case, the proteins useful in the methods of the invention and nucleic acids encoding the same as defined herein).

Specialist databases also exist for the identification of domains, for example, SMART (Schultz et al., Proc. Natl. Acad. Sci. USA 95:5857-5864, 1998; Letunic et al., Nucleic Acids Res. 30: 242-244, 2002), InterPro (Mulder et al., Nucleic Acids Res. 31:315-318, 2002), PROSITE (Bairoch and Bucher, Nucleic Acids Res., 22:3583-3589, 1994; Hofmann et al. Nucleic Acids Res., 27:215-219, 1999), or Pfam (Bateman et al., Nucleic Acids Res. 30(1):276-280, 2002). A set of tools for in silico analysis of protein sequences is available on the ExPASY proteomics server (hosted by the Swiss Institute of Bioinformatics (Gasteiger et al, Nucleic Acids Res. 31:3784-3788, 2003).

The terms “percent complementarity” or “percent complementary,” as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and an RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, a “fragment” of a polynucleotide refers to a sequence comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule or protein as disclosed herein. Fragments of a DNA molecule or protein may exhibit the activity of the DNA molecule or protein from which they are derived.

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (proA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. Plant transformation may also be carried out in the absence of selection during one or more steps or stages of culturing, developing or regenerating transformed explants, tissues, plants and/or plant parts.

### III. Transformation Methods

Methods and compositions are provided for transforming a plant cell, tissue or explant with a recombinant DNA molecule or construct encoding one or more molecules required for targeted genome editing (e.g., guide RNA(s) and/or site-directed nuclease(s)). Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are bacterially-mediated transformation, such as Agrobacterium-mediated or Rhizobium-mediated transformation, and microprojectile or particle bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616. Other methods for plant transformation, such as microinjection, electroporation, vacuum infiltration, pressure, sonication, silicon carbide fiber agitation, PEG-mediated transformation, etc., are also known in the art.

Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants. Any suitable method or technique for transformation of a plant cell known in the art may be used according to present methods. In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes.

As used herein, the terms “regeneration” and “regenerating” refer to a process of growing or developing a plant from one or more plant cells through one or more culturing steps. Transformed or edited cells, tissues or explants containing a DNA sequence insertion or edit may be grown, developed or regenerated into transgenic plants in culture, plugs, or soil according to methods known in the art. Certain embodiments of the disclosure therefore relate to methods and constructs for regenerating a plant from a cell with modified genomic DNA resulting from genome editing. The regenerated plant can then be used to propagate additional plants.

According to an aspect of the present disclosure, regenerated plants or a progeny plant, plant part or seed thereof can be screened or selected based on a marker, trait, or phenotype produced by the edit or mutation, or by the site-directed integration of an insertion sequence, transgene, etc., in the developed or regenerated plant, or a progeny plant, plant part or seed thereof. If a given mutation, edit, trait or phenotype is recessive, one or more generations or crosses (e.g., selfing) from the initial R₀ plant may be necessary to produce a plant homozygous for the edit or mutation so the trait or phenotype can be observed. Progeny plants, such as plants grown from R₁ seed or in subsequent generations, can be tested for zygosity using any known zygosity assay, such as by using a single nucleotide polymorphism (SNP) assay, DNA sequencing, thermal amplification, or polymerase chain reaction (PCR), and/or Southern blotting that allows for the distinction between heterozygote, homozygote and wild-type plants.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the use, manipulation, or analysis of a nucleic acid, a protein, or a lipid. Without being limiting, molecular techniques useful for detecting the presence of a modified sequence in a genome include phenotypic screening; molecular marker technologies such as SNP analysis by TaqMan® or Illumina/Infinium technology; Southern blot; PCR; enzyme-linked immunosorbent assay (ELISA); and sequencing (e.g., Sanger, Illumina®, 454, Pac-Bio, Ion Torrent™). In one aspect, a method of detection provided herein comprises phenotypic screening. In another aspect, a method of detection provided herein comprises SNP analysis. In a further aspect, a method of detection provided herein comprises a Southern blot. In a further aspect, a method of detection provided herein comprises PCR. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Nucleic acids can be isolated using techniques routine in the art. For example, nucleic acids can be isolated using any method including, without limitation, recombinant nucleic acid technology, and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide, northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Polypeptides can be detected using antibodies. Techniques for detecting polypeptides using antibodies include enzyme linked immunosorbent assays (ELISAs), western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

### IV. Genome Modified Plants

As used herein, “modified” in the context of a plant, plant seed, plant part, plant cell, and/or plant genome, refers to a plant, plant seed, plant part, plant cell, and/or plant genome comprising an engineered change in the expression level and/or endogenous sequence of one or more genes of interest relative to a wild-type or control plant, plant seed, plant part, plant cell, and/or plant genome. In an aspect, a modified plant, plant seed, plant part, plant cell, and/or plant genome can comprise one or more transgenes.

Modified plants, plant parts, seeds, etc., may have been subjected to mutagenesis, genome editing or site-directed integration, genetic transformation, or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity). A modified seed provided herein may give rise to a modified plant provided herein. A modified plant, plant seed, plant part, plant cell, or plant genome provided herein may comprise a recombinant DNA construct or vector or genome edit as provided herein. A “modified plant product” may be any product made from a modified plant, plant part, plant cell, or plant chromosome provided herein, or any portion or component thereof.

Modified plants may be further crossed to themselves or other plants to produce modified plant seeds and progeny. A modified plant may also be prepared by crossing a first plant comprising a DNA sequence or construct or an edit (e.g., a genomic deletion) with a second plant lacking the DNA sequence or construct or edit. For example, a DNA sequence may be introduced into a first plant line that is amenable to transformation or editing, which may then be crossed with a second plant line to introgress the DNA sequence into the second plant line. Progeny of these crosses can be further backcrossed into the desirable line multiple times, such as through 6 to 8 generations or back crosses, to produce a progeny plant with substantially the same genotype as the original parental line, but for the introduction of the DNA sequence. A modified plant, plant cell, or seed provided herein may be a hybrid plant, plant cell, or seed. As used herein, a “hybrid” is created by crossing two plants from different varieties, lines, inbreds, or species, such that the progeny comprises genetic material from each parent. Skilled artisans recognize that higher order hybrids can be generated as well.

A modified plant, plant part, plant cell, or seed provided herein may be of an elite variety or an elite line. An “elite variety” or an “elite line” refers to a variety that has resulted from breeding and selection for superior agronomic performance.

As used herein, the term “control plant” (or likewise a “control” plant seed, plant part, plant cell, and/or plant genome) refers to a plant (or plant seed, plant part, plant cell, and/or plant genome) that is used for comparison to a modified plant (or modified plant seed, plant part, plant cell, and/or plant genome) and has the same or similar genetic background (e.g., same parental lines, hybrid cross, inbred line, testers, etc.) as the modified plant (or plant seed, plant part, plant cell, and/or plant genome), except for genome edit(s). For example, a control plant may be an inbred line that is the same as the inbred line used to make the modified plant, or a control plant may be the product of the same hybrid cross of inbred parental lines as the modified plant, except for the absence in the control plant of any transgenic events or genome edit(s). Similarly, an “unmodified control plant” refers to a plant that shares a substantially similar or essentially identical genetic background as a modified plant, but without the one or more engineered changes to the genome (e.g., targeted insertion) of the modified plant. For purposes of comparison to a modified plant, plant seed, plant part, plant cell, and/or plant genome, a “wild-type plant” (or likewise a “wild-type” plant seed, plant part, plant cell, and/or plant genome) refers to a non-transgenic and non-genome edited control plant, plant seed, plant part, plant cell, and/or plant genome. As used herein, a “control” plant, plant seed, plant part, plant cell, and/or plant genome may also be a plant, plant seed, plant part, plant cell, and/or plant genome having a similar (but not the same or identical) genetic background to a modified plant, plant seed, plant part, plant cell, and/or plant genome, if deemed sufficiently similar for comparison of the characteristics or traits to be analyzed.

Modified plants comprising or derived from plant cells that are transformed with a recombinant DNA of this disclosure can be further enhanced with stacked traits, for example, a modified crop plant having an enhanced trait resulting from expression of DNA disclosed herein in combination with one or more genes of agronomic interest that provide a beneficial agronomic trait (such as herbicide and/or pest resistance traits) to crop plants. For example, the traits conferred by the recombinant DNA constructs of the current disclosure can be stacked with other traits of agronomic interest, such as a trait providing insect resistance such as using a gene from Bacillus thuringensis to provide resistance against lepidopteran, coleopteran, homopteran, hemiopteran, and other insects, or improved quality traits such as improved nutritional value. Molecules and methods for imparting insect/nematode/virus resistance are disclosed in U.S. Pat. Nos. 5,250,515; 5,880,275; 6,506,599; 5,986,175; and U.S. Patent Application Publication No. 2003/0150017 A1.

Herbicides for which transgenic plant tolerance has been demonstrated and the methods and compositions of the present disclosure can be applied include, but are not limited to, glyphosate, dicamba, glufosinate, sulfonylurea, bromoxynil, norflurazon, 2,4-D (2,4-dichlorophenoxy) acetic acid, aryloxyphenoxy propionates, and p-hydroxyphenyl pyruvate dioxygenase inhibitors (HPPD). Polynucleotide molecules encoding proteins involved in herbicide tolerance known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Patent No. Application Publication No. 2003/0083480 A1 also for imparting glyphosate tolerance; dicamba monooxygenase disclosed in U.S. Patent Application Publication No. 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al. (Plant J. 4:833-840, 1993) and in Misawa et al. (Plant J. 6:481-489, 1994) for norflurazon tolerance; a polynucleotide molecule encoding acetohydroxyacid synthase (AHAS, aka ALS) described in Sathasivan et al. (Nucl. Acids Res. 18:2188-2193, 1990) for imparting tolerance to sulfonylurea herbicides; polynucleotide molecules known as bar genes disclosed in DeBlock et al. (EMBO J. 6:2513-2519, 1987) for imparting glufosinate and bialaphos tolerance; polynucleotide molecules disclosed in U.S. Patent Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and U.S. Patent Application Publication 2002/0112260.

Genetic elements, methods, and transgenes that confer fungal disease resistance may also be used with the present disclosure (U.S. Pat. Nos. 6,653,280; 6,573,361; 6,506,962; 6,316,407; 6,215,048; 5,516,671; 5,773,696; 6,121,436; 6,316,407; 6,506,962). Soybean diseases caused by fungi include, but are not limited to, Phakopsora pachyrhizi, Phakopsora meibomiae (Asian Soybean Rust), Colletotrichum truncatum, Colletotrichum dematium var. truncatum, Glomerella glycines (Soybean Anthracnose), Phytophthora sojae (Phytophthora root and stem rot), Sclerotinia sclerotiorum (Sclerotinia stem rot), Fusarium solani f. sp. glycines (sudden death syndrome), Fusarium spp. (Fusarium root rot), Macrophomina phaseolina (charcoal rot), Septoria glycines, (Brown Spot), Pythium aphanidermatum, Pythium debaryanum, Pythium irregulare, Pythium ultimum, Pythium myriotylum, Pythium torulosum (Pythium seed decay), Diaporthe phaseolorum var. sojae (Pod blight), Phomopsis longicola (Stem blight), Phomopsis spp. (Phomopsis seed decay), Peronospora manshurica (Downy Mildew), Rhizoctonia solani (Rhizoctonia root and stem rot, Rhizoctonia aerial blight), Phialophora gregata (Brown Stem Rot), Diaporthe phaseolorum var. caulivora (Stem Canker), Cercospora kikuchii (Purple Seed Stain), Alternaria sp. (Target Spot), Cercospora sojina (Frogeye Leafspot), Sclerotium rolfsii (Southern blight), Arkoola nigra (Black leaf blight), Thielaviopsis basicola, (Black root rot), Choanephora infundibulifera, Choanephora trispora (Choanephora leaf blight), Leptosphaerulina trifolii (Leptosphaerulina leaf spot), Mycoleptodiscus terrestris (Mycoleptodiscus root rot), Neocosmospora vasinfecta (Neocosmospora stem rot), Phyllosticta sojicola (Phyllosticta leaf spot), Pyrenochaeta glycines (Pyrenochaeta leaf spot), Cylindrocladium crotalariae (Red crown rot), Dactuliochaeta glycines (Red leaf blotch), Spaceloma glycines (Scab), Stemphylium botryosum (Stemphylium leaf blight), Corynespora cassiicola (Target spot), Nematospora coryli (Yeast spot), and Phymatotrichum omnivorum (Cotton Root Rot).

### V. Definitions

The following definitions are provided to define and clarify the meaning of these terms in reference to the relevant embodiments of the present disclosure as used herein and to guide those of ordinary skill in the art in understanding the present disclosure. Unless otherwise noted, terms are to be understood according to their conventional meaning and usage in the relevant art, particularly in the field of molecular biology and plant transformation.

When introducing elements of the present disclosure or the embodiment(s) thereof, the articles “a”, “an”, “the”, and “said” are intended to mean that there are one or more of the elements.

The term “and/or”, when used in a list of two or more items, means any one of the items, any combination of the items, or all of the items with which this term is associated.

The terms “comprising”, “including”, and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

The term “about” indicates that a value includes the inherent variation of error for the method being employed to determine a value, or the variation that exists among experiments.

As used herein, a “plant” includes a whole plant, explant, plant part, seedling, or plantlet at any stage of regeneration or development. The term “plant” also includes any progeny plant. A progeny plant can be from any filial generation, e.g., F1, F2, F3, F4, F5, F6, F7, etc. The term “plant parts” include any part(s) of a plant, including, for example and without limitation: seed (including mature seed and immature seed); a plant cutting; a plant cell; a plant cell culture; a plant protoplast; a plant organ (e.g., pollen, embryos, flowers, fruits, shoots, leaves, roots, stems, and explants). A plant tissue or plant organ may be a seed, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the donor plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. A “plant part” can refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed, embryo, endosperm, seed coat, fruit, the mature ovary, propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

A plant cell is the structural and physiological unit of the plant. Plant cells, as used herein, includes protoplasts and protoplasts with a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant.

An “embryo” is a part of a plant seed, consisting of precursor tissues (e.g., meristematic tissue) that can develop into all or part of an adult plant. An “embryo” may further include a portion of a plant embryo.

A “meristem” or “meristematic tissue” comprises undifferentiated cells or meristematic cells, which are able to differentiate to produce one or more types of plant parts, tissues or structures, such as all or part of a shoot, stem, root, leaf, seed, etc.

As used herein, the “vegetative phase” of plant development is the period of growth between germination and flowering. The stages in the vegetative phase of soybean are as follows: VE (emergence), VC (cotyledon stage), V1 (first trifoliolate leaf), V2 (second trifoliolate leaf), V3 (third trifoliolate leaf), V(n) (nth trifoliolate leaf), and V6 (flowering will soon start). As used herein, the “reproductive phase” of plant development is the period between flowering and the end of harvest. The stages in the reproductive phase of soybean are as follows R1 (beginning bloom, first flower); R2 (full bloom, flower in top 2 nodes); R3 (beginning pod, 3/16″ pod in top 4 nodes); R4 (full pod, ¾″ pod in top 4 nodes); R5 (⅛″ seed in top 4 nodes); R6 (full size seed in top 4 nodes); R7 (beginning maturity, one mature pod); and, R8 (full maturity, 95% of pods on the plant have reached mature color). Soybean vegetative and reproductive stages are well known to those of skill in the art and numerous publications describing these stages can be found on the world wide web and elsewhere, such as North Dakota State University publication A-1174, June 1999, Reviewed and Reprinted August 2004.

As used herein, the term “soy” or “soybean” refers to Glycine max and includes all plant varieties that can be bred with soybean, including wild soybean species.

As used herein, the term “polynucleotide” refers to a nucleic acid molecule containing multiple nucleotides and generally comprises at least 2, at least 5, at least 10, at least 20, at least 30, at least 40, at least 50, at least 100, at least 250, at least 500, at least 1000, at least 1500, at least 2000, at least 2500, at least 3000, at least 5000, or at least 10,000 nucleotide bases. As an example, a polynucleotide provided herein can be a coding sequence or a plasmid. Shorter polynucleotides of about 18-50 nucleotides in length may be referred to as an “oligonucleotide.” Nucleic acid molecules provided herein include deoxyribonucleic acids (DNA) and ribonucleic acids (RNA) and functional analogues thereof, such as complementary DNA (cDNA). Nucleic acid molecules provided herein can be single stranded or double stranded. Nucleic acid molecules comprise the nucleotide bases adenine (A), guanine (G), thymine (T), cytosine (C). Uracil (U) replaces thymine in RNA molecules. The symbol “N” can be used to represent any nucleotide base (e.g., A, G, C, T, or U).

The term “gene expression cassette” refers to a polynucleotide sequence comprising at least a first polynucleotide sequence capable of initiating transcription of an operably linked second polynucleotide sequence and optionally a transcription termination sequence operably linked to the second polynucleotide sequence. In some aspects, the gene expression cassette may comprise a flanking left homology arm, a right homology arm, or both a left homology arm and a right homology arm.

All methods described herein can be performed in any suitable order unless otherwise indicated herein or clearly contradicted by context. The use of any and all examples, or exemplary language (e.g., “such as”) provided with respect to certain embodiments herein is intended merely to illuminate the present disclosure and does not pose a limitation on the scope of the present disclosure otherwise claimed.

Having described the present disclosure in detail, it will be apparent that modifications, variations, and equivalent embodiments are possible without departing from the spirit and scope of the present disclosure as further defined in the appended claims. Furthermore, it should be appreciated that all examples in the present disclosure including the following are provided as non-limiting examples.

### Examples

### Example 1. Selection of Soybean Genomic Target Sites and Guide RNAs

This example describes the bioinformatic analysis and identification of soybean genomic target sites for site directed integration (SDI) of transgenes; screening of LbCas12a (also known as LbCpf1) guide RNAs targeting the genomic sites and identification of unique, high-quality gRNAs to facilitate SDI of transgenes.

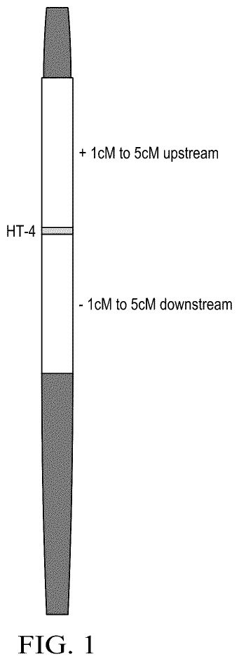

In order to identify potential soybean genomic sites for site directed integration of a selected transgene cassette at a site close to the location of the soybean GM_CSM63714 event, and generate a transgene stack to reduce transgenic loci to facilitate trait introgression and breeding, the soybean Gm_CSM63714 event insertion site was first mapped onto the Soy A3555 reference genome assembly. The genome sequence 5 centimorgan (cM) up and downstream of the event insertion site (referred to as the 10 cM window) was identified (FIG. 1) and the genetic coordinates were linked to physical positions on the Soy A3555 chromosome. The 10 cM window was subsequently interrogated for Cas12a gRNA target sites.

gRNAs and gRNA target sites for LbCas12a in the region +/−5 cM from the transgenic event were identified as follows. LbCAs12a recognizes the PAM sequence TTTV (where V is A, G, or C). Therefore, all TTTV sequences corresponding to the PAM sequences were identified and the downstream 23 nucleotide (nt) sequence corresponding to the gRNA hybridization site were identified. Those within 1 cM of the original event were removed, due to the difficulty of getting recombination between locations that are closely linked. After matching the 23 nt sequences to the genome, only those that were unique to the genome (i.e., matched the genome only a single time) were evaluated further. Next, gRNAs with potential off-targets, defined as having target sites matching the gRNA hybridization site with three or fewer mismatches and containing a TTTN PAM or NTTN PAM, where N is A, T, G or C, were removed. Target sites with corresponding gRNAs having a score of <1, as defined in PCT Patent Application Publication No. PCT/US2022/074538 (incorporated herein by referenced in its entirety) were removed from further consideration. The remaining target sites were matched to soy cDNAs and any that matched perfectly were also removed. This ensured that target sites present in coding sequence of genes were removed from consideration. The remaining 709 target sites are listed in Table 1, below.

TABLE 1
Soybean Genomic Target Sites.

| | | | | Start..End |
|---|---|---|---|---|
| | | | Start..End | of gRNA |
| Target | | | of PAM | Hybridization |
| site | Target site sequence | SEQ ID | in target | site in target |
| name | (PAM + gRNA hybridization site) | NO | site SEQ | site SEQ |
| Gm1 | TTTAGACTTATACAGCGATTAGCTCAT | 1 | 1..4 | 5..27 |
| Gm2 | TTTAGAACTTTAATTTACACTACTGGC | 2 | 1..4 | 5..27 |
| Gm3 | TTTAATAGAAGGGTCAATTGGAGGCAC | 3 | 1..4 | 5..27 |
| Gm4 | TTTAGATTGAGAAAGAGACTTCAAGCA | 4 | 1..4 | 5..27 |
| Gm5 | TTTGCATGTATAACACTATCCAAATCC | 5 | 1..4 | 5..27 |
| Gm6 | TTTGGTGATATAATTTATCCCAAACCA | 6 | 1..4 | 5..27 |
| Gm7 | TTTGGAAGCTAAACTTCTATTAGACAC | 7 | 1..4 | 5..27 |
| Gm8 | TTTCCAAACACGGCTCATATATAACGC | 8 | 1..4 | 5..27 |
| Gm9 | TTTGGTAACCACCATGGGGAACTTTGT | 9 | 1..4 | 5..27 |
| Gm10 | TTTAGAGGAATTAATTGATACCTGAAA | 10 | 1..4 | 5..27 |
| Gm11 | TTTGGATCCAGTCTATGATATAATGCG | 11 | 1..4 | 5..27 |
| Gm12 | TTTGGGAGATCAGCTGGCGGAGGAGGC | 12 | 1..4 | 5..27 |
| Gm13 | TTTAGATGAATAAACCTAGTCTGTTTC | 13 | 1..4 | 5..27 |
| Gm14 | TTTGGTGGAGAGGGTGTGATGTGAATC | 14 | 1..4 | 5..27 |
| Gm15 | TTTGGTTACGAAGGGTGCAATCACACC | 15 | 1..4 | 5..27 |
| Gm16 | TTTAGACGGTAACCCACGTGAAGGTCA | 16 | 1..4 | 5..27 |
| Gm17 | TTTAGGCTGAGGGACCAATATATATGT | 17 | 1..4 | 5..27 |
| Gm18 | TTTAGGGCTTTAGCCAAACCCGTCTAG | 18 | 1..4 | 5..27 |
| Gm19 | TTTAGCGCATGAAGATGTCTGCAAATA | 19 | 1..4 | 5..27 |
| Gm20 | TTTCGCGTGAGCCAGATATGCTCGTGA | 20 | 1..4 | 5..27 |
| Gm21 | TTTGGGAGAAAGAGTTGCAGAACACAG | 21 | 1..4 | 5..27 |
| Gm22 | TTTAGGTGAACAGAACAGAAGCTGAAA | 22 | 1..4 | 5..27 |
| Gm23 | TTTGGTGTCTCCTCCTATATAAGTAGC | 23 | 1..4 | 5..27 |
| Gm24 | TTTGGTTGTACAGATAACAGTGCCAAT | 24 | 1..4 | 5..27 |
| Gm25 | TTTGGAAATACATCATGCACCTGTAAA | 25 | 1..4 | 5..27 |
| Gm26 | TTTGGAGCATAAGGTAGCTCAAGTCTT | 26 | 1..4 | 5..27 |
| Gm27 | TTTAGAGTAATTTGAATCCCTCCATAT | 27 | 1..4 | 5..27 |
| Gm28 | TTTGGCGTTTGAAGTGATGTCACCTGG | 28 | 1..4 | 5..27 |
| Gm29 | TTTGGGAGTTGTAATGATCGCCACATA | 29 | 1..4 | 5..27 |
| Gm30 | TTTGGTACAACAATTGTATTACACTCT | 30 | 1..4 | 5..27 |
| Gm31 | TTTAGTGTATGTTTACAAGAAGATTTC | 31 | 1..4 | 5..27 |
| Gm32 | TTTAGAAAGTTGAGCTGATCATCAAAG | 32 | 1..4 | 5..27 |
| Gm33 | TTTGGAATCTTGTGACTCAGGTTACAT | 33 | 1..4 | 5..27 |
| Gm34 | TTTAGAGAGACAGAATTAAGTGAGAAA | 34 | 1..4 | 5..27 |
| Gm35 | TTTAGATAATAGATCGAGTTTACGCTC | 35 | 1..4 | 5..27 |
| Gm36 | TTTAGATCAATTCGATAGTTCGAATAA | 36 | 1..4 | 5..27 |
| Gm37 | TTTAGGCATTAAGATTGAGCTAACAAG | 37 | 1..4 | 5..27 |
| Gm38 | TTTAGGTGAATTAATTAAAGAGGGCTG | 38 | 1..4 | 5..27 |
| Gm39 | TTTCGTCCACGGTGCTCTTGAACCATT | 39 | 1..4 | 5..27 |
| Gm40 | TTTGCACCCAACAGTAGATGCCTAGGC | 40 | 1..4 | 5..27 |
| Gm41 | TTTAGAGTCTACTCTCTCCGGTCCTAT | 41 | 1..4 | 5..27 |
| Gm42 | TTTGGACCAAAGGTAGTTAATAGCTTT | 42 | 1..4 | 5..27 |
| Gm43 | TTTAGTTGCACGGTAGGTTTGGCCACC | 43 | 1..4 | 5..27 |
| Gm44 | TTTAGTAAGACCATGAAATAATCGGCC | 44 | 1..4 | 5..27 |
| Gm45 | TTTAGGTGCACCGTGGGAAGGTACTTT | 45 | 1..4 | 5..27 |
| Gm46 | TTTGGTGGAAGGACGTACTTGACGGCG | 46 | 1..4 | 5..27 |
| Gm47 | TTTCGAAATGGGGCGATCAAAGGCAGC | 47 | 1..4 | 5..27 |
| Gm48 | TTTCGGGAATTGCTTGCTGGGGCACCA | 48 | 1..4 | 5..27 |
| Gm49 | TTTGGAGAGAGAAAGTACACACGTTAA | 49 | 1..4 | 5..27 |
| Gm50 | TTTGGGCATATACAGCTTTGGAAACTC | 50 | 1..4 | 5..27 |
| Gm51 | TTTAGAGCGTTCGTGCGACAAGTTAAC | 51 | 1..4 | 5..27 |
| Gm52 | TTTGCCACTAGGGTTGCCCTCTGCCAC | 52 | 1..4 | 5..27 |
| Gm53 | TTTGGCAAGATCCGCTGCCTGCTTGAG | 53 | 1..4 | 5..27 |
| Gm54 | TTTAGCATATCAATGAACAGCCGGGTG | 54 | 1..4 | 5..27 |
| Gm55 | TTTGGTCAATGGAAGCTGTCAATATCG | 55 | 1..4 | 5..27 |
| Gm56 | TTTGGTGTATACGTGTGTGAGACAACT | 56 | 1..4 | 5..27 |
| Gm57 | TTTAGTTTCACGTCAACGACGTCCATA | 57 | 1..4 | 5..27 |
| Gm58 | TTTCGGCGGTAAGTACAACCGTAGAAG | 58 | 1..4 | 5..27 |
| Gm59 | TTTGATATAATAGTAACATTGGCAACC | 59 | 1..4 | 5..27 |
| Gm60 | TTTACTCAAACACAAATTAGAGTGGGC | 60 | 1..4 | 5..27 |
| Gm61 | TTTCGAACCAAACAGGTTGCATTGCAT | 61 | 1..4 | 5..27 |
| Gm62 | TTTGGAATTACACACTGGGTCATTGAC | 62 | 1..4 | 5..27 |
| Gm63 | TTTGGTACGCGGAAGCGAAAGCCCTAC | 63 | 1..4 | 5..27 |
| Gm64 | TTTCGTCCAACATACTTTCCTAACATC | 64 | 1..4 | 5..27 |
| Gm65 | TTTAGAGGAAGTTCCTCCCTTACCAAA | 65 | 1..4 | 5..27 |
| Gm66 | TTTGGGAGTGGTGGCTGGGAGCATTGA | 66 | 1..4 | 5..27 |
| Gm67 | TTTACTATGAATAATGTGACGAGACAC | 67 | 1..4 | 5..27 |
| Gm68 | TTTAGGGTAACATGAATTGGCCAATTT | 68 | 1..4 | 5..27 |
| Gm69 | TTTAAATCTATCTAGTCCAAATGGACC | 69 | 1..4 | 5..27 |
| Gm70 | TTTGGAGGAGTAAGAATACTCAGCACA | 70 | 1..4 | 5..27 |
| Gm71 | TTTGGTAATAATCGTCTCTGATCTTTA | 71 | 1..4 | 5..27 |
| Gm72 | TTTGGTCTCAGAAAGGCTTGGTCAACC | 72 | 1 .. | 5..27 |
| Gm73 | TTTAGAATCGTGTGGATCGTGGCCTCC | 73 | 1..4 | 5..27 |
| Gm74 | TTTAGAGCAATGGCAGCTGCACCACTG | 74 | 1..4 | 5..27 |
| Gm75 | TTTGGTTGCAGACGTAAATTTCCACGC | 75 | 1..4 | 5..27 |
| Gm76 | TTTGGAGGAGGCCACGATCCACACGAT | 76 | 1..4 | 5..27 |
| Gm77 | TTTGGGGCAAGTGGTTGACCAAGCCTT | 77 | 1..4 | 5..27 |
| Gm78 | TTTGGAATACAAGACTGACGCCGAAAC | 78 | 1..4 | 5..27 |
| Gm79 | TTTCGCCTCCTCGGCATGATTGGCCAC | 79 | 1..4 | 5..27 |
| Gm80 | TTTAGAGCTAACTCAAATTCTTGCCCA | 80 | 1..4 | 5..27 |
| Gm81 | TTTAGCAAGACAGTTGTGTGCTTTCAA | 81 | 1..4 | 5..27 |
| Gm82 | TTTGGTACTACTTTAACGTAGACAAAG | 82 | 1..4 | 5..27 |
| Gm83 | TTTAGTTACTGGGTTGAAACCAATCCT | 83 | 1..4 | 5..27 |
| Gm84 | TTTAGTTCCTGTAGCTATCAAAGGTCC | 84 | 1..4 | 5..27 |
| Gm85 | TTTGGATGTTGCTATCAACCTTCCACA | 85 | 1..4 | 5..27 |
| Gm86 | TTTAGCAGTTATCAATGCAGATCATGG | 86 | 1..4 | 5..27 |
| Gm87 | TTTCGCATCAAGGGATAATTGGCTGAT | 87 | 1..4 | 5..27 |
| Gm88 | TTTAGCTTGCTAGGATTAGGAACCAGT | 88 | 1..4 | 5..27 |
| Gm89 | TTTGGTAATATTAATATCCATGTATAC | 89 | 1..4 | 5..27 |
| Gm90 | TTTAGTATTTAACTATTGCCTCCTAAT | 90 | 1..4 | 5..27 |
| Gm91 | TTTAGTTCCTGTAGCTATCAAACTGGT | 91 | 1..4 | 5..27 |
| Gm92 | TTTGCATAAAGGACTGGCGGAATAGGC | 92 | 1..4 | 5..27 |
| Gm93 | TTTAGGGCGTACACTGTACGGATATAT | 93 | 1..4 | 5..27 |
| Gm94 | TTTACATATATCCGTACAGTGTACGCC | 94 | 1..4 | 5..27 |
| Gm95 | TTTAGAGCAATCTAGGAGTCATAATCA | 95 | 1..4 | 5..27 |
| Gm96 | TTTGGATGATGGTTAATCTCTGATTCC | 96 | 1..4 | 5..27 |
| Gm97 | TTTGGGGTTTGTATCAGTGTGTGAGAC | 97 | 1..4 | 5..27 |
| Gm98 | TTTGGTAAATTGAGGGACACTCACTCA | 98 | 1..4 | 5..27 |
| Gm99 | TTTGGTCAATGACACGTCTTCCAACTT | 99 | 1..4 | 5..27 |
| Gm100 | TTTGGAATTTCCATAGAGAGTAATACA | 100 | 1..4 | 5..27 |
| Gm101 | TTTAGACCTCTATCACACACTCAATGA | 101 | 1..4 | 5..27 |
| Gm102 | TTTCGCCCACACCATATCCCTAGGGTG | 102 | 1..4 | 5..27 |
| Gm103 | TTTGGGGATGGGTTGATTATGACTCCT | 103 | 1..4 | 5..27 |
| Gm104 | TTTGGTGGACAGTTGTTGTCATCTAGC | 104 | 1..4 | 5..27 |
| Gm105 | TTTGGTGGACTGATTGGTCGTCATAAT | 105 | 1..4 | 5..27 |
| Gm106 | TTTGGCACGCTCCACGCTGCATATATG | 106 | 1..4 | 5..27 |
| Gm107 | TTTGGTTGAGTGGACGAGTCATGCTAC | 107 | 1..4 | 5..27 |
| Gm108 | TTTCGAATAGCCCTCACTCATACAATC | 108 | 1..4 | 5..27 |
| Gm109 | TTTAATCTGAATAAATATTGGGCCACC | 109 | 1..4 | 5..27 |
| Gm110 | TTTGCAGCCAGTTTAATAATGTTTCCC | 110 | 1..4 | 5..27 |
| Gm111 | TTTCGAGAACCAAACAGAATGGGAAGC | 111 | 1..4 | 5..27 |
| Gm112 | TTTCGAGTAGAGTTTGTTCGGTCGGTC | 112 | 1..4 | 5..27 |
| Gm113 | TTTAGCAATTAGCGCAATCTCCTTTGT | 113 | 1..4 | 5..27 |
| Gm114 | TTTAGCCCAGTGTAAATAATACAATAA | 114 | 1..4 | 5..27 |
| Gm115 | TTTAGGATTGTATGAGTGAGGGCTATT | 115 | 1..4 | 5..27 |
| Gm116 | TTTGGGGACGTTTGCTCACAACAATGT | 116 | 1..4 | 5..27 |
| Gm117 | TTTGGTAACAACTGTAAATATTTATTC | 117 | 1..4 | 5..27 |
| Gm118 | TTTGGTTCCTATCGCAGTAAATTGATC | 118 | 1..4 | 5..27 |
| Gm119 | TTTGGTATAATATGGCTTCGGCCACCC | 119 | 1..4 | 5..27 |
| Gm120 | TTTGGTTAAAGTCCGTTCCGGATCAGC | 120 | 1..4 | 5..27 |
| Gm121 | TTTACGTCTATTAGGTGGCCCACTTAC | 121 | 1..4 | 5..27 |
| Gm122 | TTTGGTGACCGCATGCGACACGCAGCA | 122 | 1..4 | 5..27 |
| Gm123 | TTTGGTGCATGTGGGACCAACCAGATG | 123 | 1..4 | 5..27 |
| Gm124 | TTTGGAGAAATGAAGGAAGGGGACGAA | 124 | 1..4 | 5..27 |
| Gm125 | TTTCGAGGGCGAGACACTAAATCTGAC | 125 | 1..4 | 5..27 |
| Gm126 | TTTAGCATCACTGGATTTGTCTCCTTT | 126 | 1..4 | 5..27 |
| Gm127 | TTTGGGGCTGGCTATGACCCAAGAATA | 127 | 1..4 | 5..27 |
| Gm128 | TTTGGTGGTTGTTGAAAGCGAAGGCCA | 128 | 1..4 | 5..27 |
| Gm129 | TTTAGTTAAAGAGAATGCAACCACTAT | 129 | 1..4 | 5..27 |
| Gm130 | TTTGGTTGAATGCCCTCACCATTCACA | 130 | 1..4 | 5..27 |
| Gm131 | TTTAACAACAAACGGAAGAAAGATCAC | 131 | 1..4 | 5..27 |
| Gm132 | TTTGGACACCGGTTGCAGTAGTTGAAG | 132 | 1..4 | 5..27 |
| Gm133 | TTTGGATTGCGTAGGTGGAGGTTCTGT | 133 | 1..4 | 5..27 |
| Gm134 | TTTAGCACCGCACCATGATGATTGACA | 134 | 1..4 | 5..27 |
| Gm135 | TTTGGTGTGTTGGAATCCGAAGGAAGT | 135 | 1..4 | 5..27 |
| Gm136 | TTTACAGGCTCTGGCATATACCCACAT | 136 | 1..4 | 5..27 |
| Gm137 | TTTAGCAACAGACTAGTCTTTGACTTA | 137 | 1..4 | 5..27 |
| Gm138 | TTTAGCTGCAACTATCTCAGTTTCTGA | 138 | 1..4 | 5..27 |
| Gm139 | TTTGGGGAATTCAATCTCGATGGACAG | 139 | 1..4 | 5..27 |
| Gm140 | TTTGGTAGGTAAGGAATTAAGGGGAAG | 140 | 1..4 | 5..27 |
| Gm141 | TTTGGTCATCAATGACCCAATAAATCA | 141 | 1..4 | 5..27 |
| Gm142 | TTTGGTTTGGATTGCGTAGGTGGAGGT | 142 | 1..4 | 5..27 |
| Gm143 | TTTGGTGGAGGAAGCTTTGTTAACAAC | 143 | 1..4 | 5..27 |
| Gm144 | TTTGGAGAGTGAGTAGGCTATGAGAAA | 144 | 1..4 | 5..27 |
| Gm145 | TTTGGATAAGCACACGTCTCTTTCATC | 145 | 1..4 | 5..27 |
| Gm146 | TTTGGGTATTAACTAGCTAGTACGTAC | 146 | 1..4 | 5..27 |
| Gm147 | TTTGGTAAAGAGAAAGCATGATAAACT | 147 | 1..4 | 5..27 |
| Gm148 | TTTAGTTATAATTAGTTCCAGTTTACC | 148 | 1..4 | 5..27 |
| Gm149 | TTTGGTCACGCATCGCAGTGCCATTGC | 149 | 1..4 | 5..27 |
| Gm150 | TTTAGAAACTGCTGGACCGGTTAGTTG | 150 | 1..4 | 5..27 |
| Gm151 | TTTAGACAATGTGGAGCTGATCCAGTT | 151 | 1..4 | 5..27 |
| Gm152 | TTTGGACATATTGTGGAAATAGCTAAT | 152 | 1..4 | 5..27 |
| Gm153 | TTTCGAGCAACACGGAGGCATGTTTGG | 153 | 1..4 | 5..27 |
| Gm154 | TTTCGTATATGGAAAGAACGAAACAAC | 154 | 1..4 | 5..27 |
| Gm155 | TTTGGTGTGTATCAAATCTGGAAGGCA | 155 | 1..4 | 5..27 |
| Gm156 | TTTGGTTAGAAACAATCAATCACATGT | 156 | 1..4 | 5..27 |
| Gm157 | TTTGTATACACTCCTCATCCGGGCAGC | 157 | 1..4 | 5..27 |
| Gm158 | TTTAACAAGAGAATATAAAGCAAGGCC | 158 | 1..4 | 5..27 |
| Gm159 | TTTAACTCAAATTCTACGATCAACACC | 159 | 1..4 | 5..27 |
| Gm160 | TTTGGACCGGACATCATACTCATTCAA | 160 | 1..4 | 5..27 |
| Gm161 | TTTGGACTAAGATCCTTATCCTCGTCA | 161 | 1..4 | 5..27 |
| Gm162 | TTTAGCCACTTGACAAACTCAGATGAA | 162 | 1..4 | 5..27 |
| Gm163 | TTTAGCCCTTTCAACTGAAATATAAAT | 163 | 1..4 | 5..27 |
| Gm164 | TTTAGTGGAATTATGTGTAGGAGGGCA | 164 | 1..4 | 5..27 |
| Gm165 | TTTGTGGGTACAGGTTCAGGTAGTAGC | 165 | 1..4 | 5..27 |
| Gm166 | TTTACCCATAAGAAGATATAGGAAAGC | 166 | 1..4 | 5..27 |
| Gm167 | TTTGGACCAATTCTAAGTCTCTAACAA | 167 | 1..4 | 5..27 |
| Gm168 | TTTGGGACGACTTTATACAATCTATTT | 168 | 1..4 | 5..27 |
| Gm169 | TTTAGATCGACAAGGTTTACATGTAAG | 169 | 1..4 | 5..27 |
| Gm170 | TTTAGGTGCATACACTATATTGCCTAA | 170 | 1..4 | 5..27 |
| Gm171 | TTTGGAATTAGGAGTGAGGTTGAAGTC | 171 | 1..4 | 5..27 |
| Gm172 | TTTGGGATGAGGGCGGGGTGTGGAGGT | 172 | 1..4 | 5..27 |
| Gm173 | TTTAGCGCCGTACACTACTGTCTTCAT | 173 | 1..4 | 5..27 |
| Gm174 | TTTAGGCCAGGTCAGGTATCTGGCATT | 174 | 1..4 | 5..27 |
| Gm175 | TTTCGTATCATGACTTCAGATGTGTTC | 175 | 1..4 | 5..27 |
| Gm176 | TTTCAACGTACCTCACTAGGTTCACCC | 176 | 1..4 | 5..27 |
| Gm177 | TTTAGCGTTAATACTTCAGTACACACA | 177 | 1..4 | 5..27 |
| Gm178 | TTTGGTCACTGTTGTAGTAAGTGCTTC | 178 | 1..4 | 5..27 |
| Gm179 | TTTAGGATGGTGAGAGAATGGATGGGT | 179 | 1..4 | 5..27 |
| Gm180 | TTTGGGACAGACACAAATTCCAGAATC | 180 | 1..4 | 5..27 |
| Gm181 | TTTGGATTTAAGATGCGTAAGCTTGGT | 181 | 1..4 | 5..27 |
| Gm182 | TTTAGCATTTCTTGTCAAATGGGTGCT | 182 | 1..4 | 5..27 |
| Gm183 | TTTGGCTATCGGAATAACTGAAAGAAT | 183 | 1..4 | 5..27 |
| Gm184 | TTTGGGTGAATAACTGAATATTCTCAA | 184 | 1..4 | 5..27 |
| Gm185 | TTTAGGTGAATAAAGACAAAGAACTTC | 185 | 1..4 | 5..27 |
| Gm186 | TTTGGCAATGTCCGCATCCACATTCAC | 186 | 1..4 | 5..27 |
| Gm187 | TTTAGCTTCTATGCAACGAGGGTAAAG | 187 | 1..4 | 5..27 |
| Gm188 | TTTGGGTATGGTGAATGTGGATGCGGA | 188 | 1..4 | 5..27 |
| Gm189 | TTTAGCCGAAGATAAGTAGTTTCATTA | 189 | 1..4 | 5..27 |
| Gm190 | TTTGGATCCAGTGGGTTGAATATAGGC | 190 | 1..4 | 5..27 |
| Gm191 | TTTGGATGAATATCTATCCCAAATTAC | 191 | 1..4 | 5..27 |
| Gm192 | TTTGGTCAAGAGCGAGCAAAGACCACC | 192 | 1..4 | 5..27 |
| Gm193 | TTTAGTCAAGGTTCCGTTCCGCAGGAC | 193 | 1..4 | 5..27 |
| Gm194 | TTTAGAATCCCATGATCCTTCCCTTCC | 194 | 1..4 | 5..27 |
| Gm195 | TTTGGCCCAAAGAAATGAGCGTTGAGG | 195 | 1..4 | 5..27 |
| Gm196 | TTTAGCTGTATCCAGGCTAACTCCAAA | 196 | 1..4 | 5..27 |
| Gm197 | TTTAGTAGCGCAAAGTAGCATGCACGT | 197 | 1..4 | 5..27 |
| Gm198 | TTTAGTAGGCCCTGGCAGAGCCAAGGA | 198 | 1..4 | 5..27 |
| Gm199 | TTTAAATGAAAGTGTCGAGGTCCACAC | 199 | 1..4 | 5..27 |
| Gm200 | TTTGGAACTGCAGCACGAGCAGTTAAG | 200 | 1..4 | 5..27 |
| Gm201 | TTTGGAGTTAGCCTGGATACAGCTAAA | 201 | 1..4 | 5..27 |
| Gm202 | TTTCGATCTGTGGCTCCGCGCGCAGAT | 202 | 1..4 | 5..27 |
| Gm203 | TTTAGCTTGTCTCCTCGCTAGTATATC | 203 | 1..4 | 5..27 |
| Gm204 | TTTAAACGGACTGAATCATGTATAGGC | 204 | 1..4 | 5..27 |
| Gm205 | TTTACCGAGACGGATAATATCTTCTTC | 205 | 1..4 | 5..27 |
| Gm206 | TTTACGCGACGACGCCAAAGTCACTCC | 206 | 1..4 | 5..27 |
| Gm207 | TTTAGAGTCTAGAGACGTGTCATGCAA | 207 | 1..4 | 5..27 |
| Gm208 | TTTGGCGTCGTCGCGTAAATTGGGGTG | 208 | 1..4 | 5..27 |
| Gm209 | TTTGGGCAAGGAATTAGCATGCTCATT | 209 | 1..4 | 5..27 |
| Gm210 | TTTAGGTGATGACATCTACAATTTCAC | 210 | 1..4 | 5..27 |
| Gm211 | TTTGGTACTAGAACCGTCTGAGTTGAG | 211 | 1..4 | 5..27 |
| Gm212 | TTTAGTTCAACTCTGGTATGTTACTCT | 212 | 1..4 | 5..27 |
| Gm213 | TTTAGAAATTGGAATTAGCTCACACAA | 213 | 1..4 | 5..27 |
| Gm214 | TTTAGAATCATTCCATTCCATTCAGTC | 214 | 1..4 | 5..27 |
| Gm215 | TTTGGACTACATGAGTACTCTGAATCA | 215 | 1..4 | 5..27 |
| Gm216 | TTTGGATTCGCTATTGCGATACCGGGT | 216 | 1..4 | 5..27 |
| Gm217 | TTTAGATTGAGAATTGAACGAAATGAG | 217 | 1..4 | 5..27 |
| Gm218 | TTTGGCTTCGTAACTTCAATGGAGATC | 218 | 1..4 | 5..27 |
| Gm219 | TTTGGGAATCCTAAGTCGAACCGAACA | 219 | 1..4 | 5..27 |
| Gm220 | TTTCGGAGTGGCTCAACTCAGACGGTT | 220 | 1..4 | 5..27 |
| Gm221 | TTTGGGGCTTCTTTCCCTAGAGGGGTA | 221 | 1..4 | 5..27 |
| Gm222 | TTTAGGTGATAGGTAGGTCTTGCTTTG | 222 | 1..4 | 5..27 |
| Gm223 | TTTAGTACGCATAAAGCTTCTCCCTCT | 223 | 1..4 | 5..27 |
| Gm224 | TTTAGTATGTAGTTTATTATGCATCGC | 224 | 1..4 | 5..27 |
| Gm225 | TTTAGTTGAATCAGCAAAGACTTTGAT | 225 | 1..4 | 5..27 |
| Gm226 | TTTATAGCAATGAGGCTTCCGTGTCAC | 226 | 1..4 | 5..27 |
| Gm227 | TTTATGTTGATAGGTACACCCAGACCC | 227 | 1..4 | 5..27 |
| Gm228 | TTTAGCAACGGCCACGACCTACTCCTC | 228 | 1..4 | 5..27 |
| Gm229 | TTTGGGCAAAGAAAGGTCTTGAATTGC | 229 | 1..4 | 5..27 |
| Gm230 | TTTGGGAGAATCTTTGGTGGATGTTGC | 230 | 1..4 | 5..27 |
| Gm231 | TTTGGGAAGGGCGTAGGGTATGGTTGC | 231 | 1..4 | 5..27 |
| Gm232 | TTTAGCTGAACAACGTCTAGGATGTAT | 232 | 1..4 | 5..27 |
| Gm233 | TTTGGTCACATCAAGTGCGACAGGTAT | 233 | 1..4 | 5..27 |
| Gm234 | TTTGGACACGTGTCAGCATCCGAGAAG | 234 | 1..4 | 5..27 |
| Gm235 | TTTAGAGGCAGCCCATATATTCACTTG | 235 | 1..4 | 5..27 |
| Gm236 | TTTAGGCACAGAAGTTTAAGGAACTTT | 236 | 1..4 | 5..27 |
| Gm237 | TTTGGGGTGTTGCTCTCAGGAGTGATT | 237 | 1..4 | 5..27 |
| Gm238 | TTTGGACACCCTCACAAACAATACAAT | 238 | 1..4 | 5..27 |
| Gm239 | TTTCGACAGCGACGACAAAGACAACGT | 239 | 1..4 | 5..27 |
| Gm240 | TTTGGATTTGACGGAGAGGCAGGTGAG | 240 | 1..4 | 5..27 |
| Gm241 | TTTAGCATATCCGAATATAGCTCCGCA | 241 | 1..4 | 5..27 |
| Gm242 | TTTGGTGATTCTCTAATTGCCATCCCT | 242 | 1..4 | 5..27 |
| Gm243 | TTTAGTGTCACTGGCTAGCAACTGATT | 243 | 1..4 | 5..27 |
| Gm244 | TTTAGAAACTCTAACAACTCGGTTAAA | 244 | 1..4 | 5..27 |
| Gm245 | TTTGGTGAATGGTGGGGTACAGTTATA | 245 | 1..4 | 5..27 |
| Gm246 | TTTGGTGGATGTTGCATGATGAGTTGT | 246 | 1..4 | 5..27 |
| Gm247 | TTTGGTGTGAACTAAGAGTGTAGTATT | 247 | 1..4 | 5..27 |
| Gm248 | TTTGGTTGAATATGTTTAGGGGCGGAA | 248 | 1..4 | 5..27 |
| Gm249 | TTTGGAGCCAATAACCGTGATACATTC | 249 | 1..4 | 5..27 |
| Gm250 | TTTGGAGAAATAAGATACAAGCAATTC | 250 | 1..4 | 5..27 |
| Gm251 | TTTAGAGAGAAGTTAAATGGAAGCATC | 251 | 1..4 | 5..27 |
| Gm252 | TTTAGTCCATCCCTAAGGAGGCAACCA | 252 | 1..4 | 5..27 |
| Gm253 | TTTGGGGCTCCGGTCCATTAAATAAGC | 253 | 1..4 | 5..27 |
| Gm254 | TTTGGGAATTCCTTTAACAGGTAAGGC | 254 | 1..4 | 5..27 |
| Gm255 | TTTAGTAAATTTAACACGCACCGTACC | 255 | 1..4 | 5..27 |
| Gm256 | TTTGGTGCCAGGTTATGAATGGAAAGT | 256 | 1..4 | 5..27 |
| Gm257 | TTTGGAATGGGCAAAGATCCGACATGT | 257 | 1..4 | 5..27 |
| Gm258 | TTTAGGGAAGGATAGATATAACATTTC | 258 | 1..4 | 5..27 |
| Gm259 | TTTAGAATTGTGATATATCACCTCATC | 259 | 1..4 | 5..27 |
| Gm260 | TTTAGACAACATAATAATACGAATTGC | 260 | 1..4 | 5..27 |
| Gm261 | TTTGGCATCCTAGATTTGGCTCCACTT | 261 | 1..4 | 5..27 |
| Gm262 | TTTAGGAGCCTTACCTGTTAAAGGAAT | 262 | 1..4 | 5..27 |
| Gm263 | TTTGGACATATATGCTATATCATTCTC | 263 | 1..4 | 5..27 |
| Gm264 | TTTGGGGTGGCCACCACATATACCAAC | 264 | 1..4 | 5..27 |
| Gm265 | TTTGGGCACCTGCTCTATCATGCAGAC | 265 | 1..4 | 5..27 |
| Gm266 | TTTGGCTGCTGCAGACACCGTTAAGTT | 266 | 1..4 | 5..27 |
| Gm267 | TTTGGCTGGGTTCCTGGGGAAGTCACA | 267 | 1..4 | 5..27 |
| Gm268 | TTTGGTCTAATAATAATTTGGGGTGGC | 268 | 1..4 | 5..27 |
| Gm269 | TTTGGATGACAACTATCAGTAACACCG | 269 | 1..4 | 5..27 |
| Gm270 | TTTAGGCAGCCATGGTGAAGGAATATT | 270 | 1..4 | 5..27 |
| Gm271 | TTTGATCAAAGCTAAAGCATAAGCATC | 271 | 1..4 | 5..27 |
| Gm272 | TTTAGAAGTTGCATGATGATTGTCGGT | 272 | 1..4 | 5..27 |
| Gm273 | TTTGGCAATGTTAGTTGGAGTGCAACT | 273 | 1..4 | 5..27 |
| Gm274 | TTTGGCTCCGCTACCCAGATAAATGAA | 274 | 1..4 | 5..27 |
| Gm275 | TTTACAAGGATCACAGCACCCTCAGAC | 275 | 1..4 | 5..27 |
| Gm276 | TTTAGGAGCTTTGGGGCCTCGATTTAC | 276 | 1..4 | 5..27 |
| Gm277 | TTTAGCATCTATGACACCATGAGTATC | 277 | 1..4 | 5..27 |
| Gm278 | TTTAGCATGGCTCATCAATTGCCAGCT | 278 | 1..4 | 5..27 |
| Gm279 | TTTAGCTATATTCAGCAAAGCCCTCCA | 279 | 1..4 | 5..27 |
| Gm280 | TTTGGGTCAAGAAGATGTATGGGTGAA | 280 | 1..4 | 5..27 |
| Gm281 | TTTGGACTTAGCATAAACTGAGGTGAG | 281 | 1..4 | 5..27 |
| Gm282 | TTTGGAGTCTAGTAAGCTTCTAAAGAC | 282 | 1..4 | 5..27 |
| Gm283 | TTTGGGAGGTCACGAAGATGCTTATAA | 283 | 1..4 | 5..27 |
| Gm284 | TTTAGGGAGCTGGGGTGTTGCATTTAA | 284 | 1..4 | 5..27 |
| Gm285 | TTTAGGTATACTAATGAGTATGCTCCA | 285 | 1..4 | 5..27 |
| Gm286 | TTTCGATGCAGATCTGGACTTGGGTGA | 286 | 1..4 | 5..27 |
| Gm287 | TTTGGGCCAGCGAAATAAATAGTTAAT | 287 | 1..4 | 5..27 |
| Gm288 | TTTGGGCACAGCACCAACCCTAGTGAC | 288 | 1..4 | 5..27 |
| Gm289 | TTTGGGTACACCCAGTAGCGTGGTCCT | 289 | 1..4 | 5..27 |
| Gm290 | TTTGGCTACAAAGGCACCCAGTCCACA | 290 | 1..4 | 5..27 |
| Gm291 | TTTAGTTTAATCCTCTAGTACTTCTCC | 291 | 1..4 | 5..27 |
| Gm292 | TTTAGCTCTTCTGTCTTGGAGGATACC | 292 | 1..4 | 5..27 |
| Gm293 | TTTGGAGTAAGCAATGTGCAGGTCAAG | 293 | 1..4 | 5..27 |
| Gm294 | TTTAGATGGGCTATCAGCACAGCAGCT | 294 | 1..4 | 5..27 |
| Gm295 | TTTGGAACCAAGTGTACGTGATCTATA | 295 | 1..4 | 5..27 |
| Gm296 | TTTGGGCCTAATGGGCCTCAATTTACA | 296 | 1..4 | 5..27 |
| Gm297 | TTTGGACACTTGCTATATTCGCTCCCT | 297 | 1..4 | 5..27 |
| Gm298 | TTTGGAGTTCTTGCTCTCGGGGAAGCT | 298 | 1..4 | 5..27 |
| Gm299 | TTTAGGGTAGCACAACCTTAACTCATT | 299 | 1..4 | 5..27 |
| Gm300 | TTTGGTTGACACCATAACCCTATTCGC | 300 | 1..4 | 5..27 |
| Gm301 | TTTAACGCAAACGCTTAGTGATTTAAC | 301 | 1..4 | 5..27 |
| Gm302 | TTTAATGCAAAGAATAAGGCTTGGCAC | 302 | 1..4 | 5..27 |
| Gm303 | TTTGCATGGATATATAGCTTCTGCAGC | 303 | 1..4 | 5..27 |
| Gm304 | TTTACGGAAAGGGCCGTGTGGACTGGG | 304 | 1..4 | 5..27 |
| Gm305 | TTTAGATTTCTGTATGTGGGACAATTC | 305 | 1..4 | 5..27 |
| Gm306 | TTTAGGTCAAATTTGGTTGACACCATA | 306 | 1..4 | 5..27 |
| Gm307 | TTTGGAGGAACAATTGTGCATTTAGCC | 307 | 1..4 | 5..27 |
| Gm308 | TTTGGGCCGGGTGGAAACAATGGAGCC | 308 | 1..4 | 5..27 |
| Gm309 | TTTGGGACCAAGCCACCTACTAGGCTA | 309 | 1..4 | 5..27 |
| Gm310 | TTTGGAGACAACTAAACATGAAGCGTG | 310 | 1..4 | 5..27 |
| Gm311 | TTTAGAAACAAGTTCGACTTCCGGATG | 311 | 1..4 | 5..27 |
| Gm312 | TTTAGGATGAGGAGAACTCTTAAAGTT | 312 | 1..4 | 5..27 |
| Gm313 | TTTGGTTGTGGGGTTGCAAATGTAAAC | 313 | 1..4 | 5..27 |
| Gm314 | TTTGGAACGGACTACACATGACTTAAG | 314 | 1..4 | 5..27 |
| Gm315 | TTTGGCGTTTGCAAATACTCTTCTAAT | 315 | 1..4 | 5..27 |
| Gm316 | TTTCGTCCAATTTAAACAAGCCCTCAG | 316 | 1..4 | 5..27 |
| Gm317 | TTTAGTGAAATAACTTGTCGAATATCG | 317 | 1..4 | 5..27 |
| Gm318 | TTTAGGTACAAAGTATTAAGCTGCATC | 318 | 1..4 | 5..27 |
| Gm319 | TTTACATTGAATGAGATTGGCTAGGAC | 319 | 1..4 | 5..27 |
| Gm320 | TTTAGGGTTGTTGACGATATGAATCAC | 320 | 1..4 | 5..27 |
| Gm321 | TTTGGTAATAGCACACCACAATGTGTT | 321 | 1..4 | 5..27 |
| Gm322 | TTTAGGCTGAATAAGCAGAAAGGAATC | 322 | 1..4 | 5..27 |
| Gm323 | TTTGGAAGTATCACATCCTGTTTATTC | 323 | 1..4 | 5..27 |
| Gm324 | TTTGGCGTGGAGGCATCATCACATCAC | 324 | 1..4 | 5..27 |
| Gm325 | TTTAGCTCCTTACAGCAGAGGTAGCCC | 325 | 1..4 | 5..27 |
| Gm326 | TTTAGGGTGGTAGCCTGCACTAGTCCC | 326 | 1..4 | 5..27 |
| Gm327 | TTTAGATGTAACTACATTCGTAACTGC | 327 | 1..4 | 5..27 |
| Gm328 | TTTGGGATTATCCATGTATGATGTTGC | 328 | 1..4 | 5..27 |
| Gm329 | TTTGGGCCGAATTTGAGCTGCAATCTG | 329 | 1..4 | 5..27 |
| Gm330 | TTTGGTGGGTTGGGGTTGGGGACCGAT | 330 | 1..4 | 5..27 |
| Gm331 | TTTGGTTTGACATAGTAGTTGTAAAGC | 331 | 1..4 | 5..27 |
| Gm332 | TTTGGCGGAGTCGAGTCTGAGTACATG | 332 | 1..4 | 5..27 |
| Gm333 | TTTGGATCCAACAGGACAGATCAAGAA | 333 | 1..4 | 5..27 |
| Gm334 | TTTCGTAGATCTCCACTCAACGTATAC | 334 | 1..4 | 5..27 |
| Gm335 | TTTCGTTCATAACAGTGAGGGAACGGC | 335 | 1..4 | 5..27 |
| Gm336 | TTTCGTTGATGGCGTCACAAGCAAGCC | 336 | 1..4 | 5..27 |
| Gm337 | TTTAGGAACAAGATTGGGAATGGGTCA | 337 | 1..4 | 5..27 |
| Gm338 | TTTGGTTGCATTTGCATGGATAAACCT | 338 | 1..4 | 5..27 |
| Gm339 | TTTACCAAGAGTTTATAACAGGATGAC | 339 | 1..4 | 5..27 |
| Gm340 | TTTAGAAATAGAAGGTTCCCTCTTGTG | 340 | 1..4 | 5..27 |
| Gm341 | TTTAGCCTATATGATGGACACATGTAT | 341 | 1..4 | 5..27 |
| Gm342 | TTTGGGCATACAGTATCTAATAACTTA | 342 | 1..4 | 5..27 |
| Gm343 | TTTAGTCTCAGTAATGAAGTTTGAACT | 343 | 1..4 | 5..27 |
| Gm344 | TTTGGCTTCAGCTAAGAAGGGACGAGA | 344 | 1..4 | 5..27 |
| Gm345 | TTTGGGCCTTTGGGCCTACCATAATGG | 345 | 1..4 | 5..27 |
| Gm346 | TTTAGTGTAGATAAGGTATCAGGTCAC | 346 | 1..4 | 5..27 |
| Gm347 | TTTGCATACACGCGTACAATTGAAATC | 347 | 1..4 | 5..27 |
| Gm348 | TTTGGACAAGTACTACACTGAACATCT | 348 | 1..4 | 5..27 |
| Gm349 | TTTAGATGCGCATAGATACAGAATTGC | 349 | 1..4 | 5..27 |
| Gm350 | TTTGGGCCTACCATAATGGAAATTATT | 350 | 1..4 | 5..27 |
| Gm351 | TTTAGGCTAAATAGACATCAACACTTG | 351 | 1..4 | 5..27 |
| Gm352 | TTTGGATAAATTACATCTACTGAGCAT | 352 | 1..4 | 5..27 |
| Gm353 | TTTAGCATGATAAATTTGTGAGAACTA | 353 | 1..4 | 5..27 |
| Gm354 | TTTAGTTGAAATTAATGCTCAGTAGAT | 354 | 1..4 | 5..27 |
| Gm355 | TTTAGTGCGCATTCGCTACGTACTCGC | 355 | 1..4 | 5..27 |
| Gm356 | TTTGGAGCATGGGCTGTGTAAGACTTG | 356 | 1..4 | 5..27 |
| Gm357 | TTTGGGTGTATACATGCCACACATGAT | 357 | 1..4 | 5..27 |
| Gm358 | TTTGGTACACTGAACATGCAATCCACC | 358 | 1..4 | 5..27 |
| Gm359 | TTTAAGAGAAGGGGTCAGTATAATCCC | 359 | 1..4 | 5..27 |
| Gm360 | TTTGGTGTACCAGTGGAAGGACAAGAG | 360 | 1..4 | 5..27 |
| Gm361 | TTTAGTGAGATGTCATGTAAGATAAGT | 361 | 1..4 | 5..27 |
| Gm362 | TTTACACATATCCAGTAGAGATTTATC | 362 | 1..4 | 5..27 |
| Gm363 | TTTGGATTTAGTGTCCAAACTAATACA | 363 | 1..4 | 5..27 |
| Gm364 | TTTGGGAATTTGATATCCCATTTAGTC | 364 | 1..4 | 5..27 |
| Gm365 | TTTGGGCTGAACTCGAGAGAATAACCC | 365 | 1..4 | 5..27 |
| Gm366 | TTTAGACCAACACGACGTCGTGAATAT | 366 | 1..4 | 5..27 |
| Gm367 | TTTGGCACCTAACATAAACTCAGCTGT | 367 | 1..4 | 5..27 |
| Gm368 | TTTAGTGGCCTGGGCAGTAAATAAGCA | 368 | 1..4 | 5..27 |
| Gm369 | TTTGGGTTTATATGAGGGTAATTAACT | 369 | 1..4 | 5..27 |
| Gm370 | TTTCGTGCTGGTCGTATGACACGAGAT | 370 | 1..4 | 5..27 |
| Gm371 | TTTCAGCCCATAGCAAGTTTAGTGGCC | 371 | 1..4 | 5..27 |
| Gm372 | TTTAGAATAACCAACAATTAGTGGTTA | 372 | 1..4 | 5..27 |
| Gm373 | TTTAGGTATTTATCCTAAAGAGACATT | 373 | 1..4 | 5..27 |
| Gm374 | TTTGGTCAATTGTACGATATGTCTCAT | 374 | 1..4 | 5..27 |
| Gm375 | TTTGGTCTCTCTTCTAATATCACTAAT | 375 | 1..4 | 5..27 |
| Gm376 | TTTCGTGTTGAACAAACACAGGTACAC | 376 | 1..4 | 5..27 |
| Gm377 | TTTGCACTGACGCAAACAAGAATTCTC | 377 | 1..4 | 5..27 |
| Gm378 | TTTAGGGGAACTCCATTTGAATTTCCT | 378 | 1..4 | 5..27 |
| Gm379 | TTTAGCTAGTTTGGTCTTGCGGTGTAG | 379 | 1..4 | 5..27 |
| Gm380 | TTTGGGACATCGAACAGCTAATAAATT | 380 | 1..4 | 5..27 |
| Gm381 | TTTGGTCTTATCTACGGCTCTTGATTC | 381 | 1 .. | 5..27 |
| Gm382 | TTTCGCCTCAATGAACCAAGTAAGGAA | 382 | 1..4 | 5..27 |
| Gm383 | TTTGGGAACTCCATGTGAATTTCCATG | 383 | 1..4 | 5..27 |
| Gm384 | TTTCGTATGAAGACGCCAATTATCAAT | 384 | 1..4 | 5..27 |
| Gm385 | TTTGGTGACACTTAATTAGTACAAGGA | 385 | 1..4 | 5..27 |
| Gm386 | TTTGGATCAATTTGATCTGGAAGAGAC | 386 | 1..4 | 5..27 |
| Gm387 | TTTAGCGAGGCGGGTTAGGATTTCAAC | 387 | 1..4 | 5..27 |
| Gm388 | TTTAGCCTCCAACCATGTCAAGACTTG | 388 | 1..4 | 5..27 |
| Gm389 | TTTAGAAAGTGAGCTGGTATTATTTCC | 389 | 1..4 | 5..27 |
| Gm390 | TTTAGAAGCGTGAGACTTGACGTGATG | 390 | 1..4 | 5..27 |
| Gm391 | TTTAGATCAAGATGAGTACAGAAGAAA | 391 | 1..4 | 5..27 |
| Gm392 | TTTGGCATATCAATAAATAACATGGCT | 392 | 1..4 | 5..27 |
| Gm393 | TTTAGCCCAGTCCAATTAAGTCGTAGG | 393 | 1..4 | 5..27 |
| Gm394 | TTTGGAAATATTAAGGGATTGAGGATA | 394 | 1..4 | 5..27 |
| Gm395 | TTTGGAATAACTAAAGTGATCTTCGCA | 395 | 1..4 | 5..27 |
| Gm396 | TTTGGTTCCCTTCACTCTCACAATGCG | 396 | 1..4 | 5..27 |
| Gm397 | TTTAGTTGATAACACACTCGGACCAGA | 397 | 1..4 | 5..27 |
| Gm398 | TTTGATGTGAACTGCATACACCGCCGC | 398 | 1..4 | 5..27 |
| Gm399 | TTTGGATTGTGAAGCTGGTAACCCGCT | 399 | 1..4 | 5..27 |
| Gm400 | TTTAACACCATTACTAGTGTGGCTGCC | 400 | 1..4 | 5..27 |
| Gm401 | TTTGGAAATTAGGAGTGCAGGGTACGT | 401 | 1..4 | 5..27 |
| Gm402 | TTTGGATAAACGGTAATGTCTCTTTAT | 402 | 1..4 | 5..27 |
| Gm403 | TTTAGGACCAAATTTGTACAAACTTAG | 403 | 1..4 | 5..27 |
| Gm404 | TTTAGGGAACTTGTAACAATTAGAGCC | 404 | 1..4 | 5..27 |
| Gm405 | TTTAAGGTGAGAATACACAAATAAGTC | 405 | 1..4 | 5..27 |
| Gm406 | TTTCCAAGCTGTCTCCGGCGCCTAATC | 406 | 1..4 | 5..27 |
| Gm407 | TTTGCTGTAATTTGCTAGTCTCGTTGC | 407 | 1..4 | 5..27 |
| Gm408 | TTTAGATAACGGGGCTATAGTCCATTG | 408 | 1..4 | 5..27 |
| Gm409 | TTTGGGAAATATGAGTTGAATGCCTTT | 409 | 1..4 | 5..27 |
| Gm410 | TTTCGGCCTCGATGATGAATATATAAC | 410 | 1..4 | 5..27 |
| Gm411 | TTTGGTAAATGGGAAATAGAGATGAGA | 411 | 1..4 | 5..27 |
| Gm412 | TTTGGTTGCTATACGTGTCCGCATAAA | 412 | 1..4 | 5..27 |
| Gm413 | TTTGTAACCACCTTGAGCAACTCGAGC | 413 | 1..4 | 5..27 |
| Gm414 | TTTACTGCTAGCAGCCATTCTCTCAAC | 414 | 1..4 | 5..27 |
| Gm415 | TTTAGGACTAGCCAACATCATACCATA | 415 | 1..4 | 5..27 |
| Gm416 | TTTGGCGCGTGTCTTCTTCACGCTCAA | 416 | 1..4 | 5..27 |
| Gm417 | TTTGGGTTTATGCTATAGTAGTTATCT | 417 | 1..4 | 5..27 |
| Gm418 | TTTGGTAGTTTACTCTGTACTATTACC | 418 | 1..4 | 5..27 |
| Gm419 | TTTGGCGCGACTTCAATGATGCCGCTC | 419 | 1..4 | 5..27 |
| Gm420 | TTTAGCTCAAGTCTTTGGTGTACGTGC | 420 | 1..4 | 5..27 |
| Gm421 | TTTGGGGTCAGCTTATCACGTAAAGAC | 421 | 1..4 | 5..27 |
| Gm422 | TTTAGGGTACGCACCCTAATCTGTCTC | 422 | 1..4 | 5..27 |
| Gm423 | TTTCGTAGAAATAACACGTGCCGATTC | 423 | 1..4 | 5..27 |
| Gm424 | TTTGGTATGATATTGTAACCACGTCAC | 424 | 1..4 | 5..27 |
| Gm425 | TTTGGACACCCTCCGCAGGATCTAGCG | 425 | 1..4 | 5..27 |
| Gm426 | TTTGGACACCTTTGAAGAGGATCCAGC | 426 | 1..4 | 5..27 |
| Gm427 | TTTGGGAGGTTGTACAAAGGATTTCCC | 427 | 1..4 | 5..27 |
| Gm428 | TTTCGTCATAACACAGAGTAGTTATGC | 428 | 1..4 | 5..27 |
| Gm429 | TTTGGTGTACGTGCCCATCCGAAATAG | 429 | 1..4 | 5..27 |
| Gm430 | TTTAGCAGGTTTCGCTAGTCAAACAGG | 430 | 1..4 | 5..27 |
| Gm431 | TTTCGCTAGTCAAACAGGTCTGTCACG | 431 | 1..4 | 5..27 |
| Gm432 | TTTAGGAATGTGCAATGAATGCGTAAC | 432 | 1..4 | 5..27 |
| Gm433 | TTTCGGATGGGCACGTACACCAAAGAC | 433 | 1..4 | 5..27 |
| Gm434 | TTTGGTGATATAAATGTGGACTCTCAT | 434 | 1..4 | 5..27 |
| Gm435 | TTTAACATTATGATGATCGGACTCACC | 435 | 1..4 | 5..27 |
| Gm436 | TTTAGACATAGTTACCACACTACTTCT | 436 | 1..4 | 5..27 |
| Gm437 | TTTAGTAATCGGGATCAATCTCAAACT | 437 | 1..4 | 5..27 |
| Gm438 | TTTAGTCTTTACGTGATAAGCTGACCC | 438 | 1..4 | 5..27 |
| Gm439 | TTTAGTGGGAATTAGATGTACAGAGAT | 439 | 1..4 | 5..27 |
| Gm440 | TTTAGTTGCTCCAACTTGTATATGACC | 440 | 1..4 | 5..27 |
| Gm441 | TTTAAACATACATTGTAGGGTGAGTCC | 441 | 1..4 | 5..27 |
| Gm442 | TTTGCAACCTCCTATTGCTAGCCAACC | 442 | 1..4 | 5..27 |
| Gm443 | TTTGCTGTTATGGTAGGTTGGAAAGGC | 443 | 1..4 | 5..27 |
| Gm444 | TTTAGAGAGATGGTTAAGTTATCATAG | 444 | 1..4 | 5..27 |
| Gm445 | TTTGGAGTTTGGACACCTTTGAAGAGG | 445 | 1..4 | 5..27 |
| Gm446 | TTTGGATAGGTGTATGTGTGATCCAAG | 446 | 1..4 | 5..27 |
| Gm447 | TTTAGTCACTTTATGTTGGACTGCCCT | 447 | 1..4 | 5..27 |
| Gm448 | TTTAGTCTTCCATCTTCATCTATATCC | 448 | 1..4 | 5..27 |
| Gm449 | TTTAGTGCAATATTCGTACTTCCAGGA | 449 | 1..4 | 5..27 |
| Gm450 | TTTAGTGTCAGTGTCACAAGCGGACAT | 450 | 1..4 | 5..27 |
| Gm451 | TTTAGAAGAACTAGCTAGCCTAATCCG | 451 | 1..4 | 5..27 |
| Gm452 | TTTCGGTGTGCCGCCGACGTCGATTCC | 452 | 1..4 | 5..27 |
| Gm453 | TTTGGGTTGCAGTGCATGTGCAGTGGC | 453 | 1..4 | 5..27 |
| Gm454 | TTTAGAGTGTATGATTGTGTACCTACC | 454 | 1..4 | 5..27 |
| Gm455 | TTTAGCAGTTAGAGGGCAGTCGTAGCA | 455 | 1..4 | 5..27 |
| Gm456 | TTTAGGGATATATACGTGAGTCAGAGG | 456 | 1..4 | 5..27 |
| Gm457 | TTTGGACTATGTTGTGTTTCCTCGTAC | 457 | 1..4 | 5..27 |
| Gm458 | TTTGGCCACAAATTTCGATAGTTCAAA | 458 | 1..4 | 5..27 |
| Gm459 | TTTGGGTTTATTATGCTCTCTTGGTCC | 459 | 1..4 | 5..27 |
| Gm460 | TTTAGTTTAATCCCTTAGACGATAAAT | 460 | 1..4 | 5..27 |
| Gm461 | TTTCCGGCAACAACATTATGCTTTCAC | 461 | 1..4 | 5..27 |
| Gm462 | TTTAGAAACTCCAACGAATTATTACTG | 462 | 1..4 | 5..27 |
| Gm463 | TTTAGCAAATTAACCGGCTGGCTTTGA | 463 | 1..4 | 5..27 |
| Gm464 | TTTAGTATCAGTATGAGTCTGAGATAG | 464 | 1..4 | 5..27 |
| Gm465 | TTTAGTCATGTCCGCTTGTGACACTGA | 465 | 1..4 | 5..27 |
| Gm466 | TTTGGGGTGGCAAAGGAATGGAGACTC | 466 | 1..4 | 5..27 |
| Gm467 | TTTAGAGAGACAAGAACTGTGCCCAAA | 467 | 1..4 | 5..27 |
| Gm468 | TTTGGTGCGGCAGAAACAGAGGAAGAA | 468 | 1..4 | 5..27 |
| Gm469 | TTTAGAGGTATCTAAGTAGAGAAGTCA | 469 | 1..4 | 5..27 |
| Gm470 | TTTGGGCACAGTTCTTGTCTCTCTAAA | 470 | 1..4 | 5..27 |
| Gm471 | TTTAATCCAAATTTGCACAAGATAATC | 471 | 1..4 | 5..27 |
| Gm472 | TTTGGATTAAACTTTATGGGTGCACGT | 472 | 1..4 | 5..27 |
| Gm473 | TTTAGCATAAATTATATACAATTTAAC | 473 | 1..4 | 5..27 |
| Gm474 | TTTGGGTCTATATATATGACAACTGAA | 474 | 1..4 | 5..27 |
| Gm475 | TTTGGTGCTAATTAGATCAGATTGGAT | 475 | 1..4 | 5..27 |
| Gm476 | TTTAGGCAATGGGGTTCCGAAGAAGAT | 476 | 1..4 | 5..27 |
| Gm477 | TTTAGCTAGAAGAACACCCAAGATGCA | 477 | 1..4 | 5..27 |
| Gm478 | TTTGGGTCTTCGTGGCAGAAGGTGTTA | 478 | 1..4 | 5..27 |
| Gm479 | TTTACCGGTTTAGGCATGTGTACCCAC | 479 | 1..4 | 5..27 |
| Gm480 | TTTGCTGCAAACTCCTTTAATAGAAAC | 480 | 1..4 | 5..27 |
| Gm481 | TTTGGAAAGTAATTATGTAGCCAAATG | 481 | 1..4 | 5..27 |
| Gm482 | TTTCGAATGATGAACTGAAACCATAAA | 482 | 1..4 | 5..27 |
| Gm483 | TTTGGTGCAAACTAACTTGCTACAGAA | 483 | 1..4 | 5..27 |
| Gm484 | TTTAGAATATTGGTGTCAAGACCCGGC | 484 | 1..4 | 5..27 |
| Gm485 | TTTGCTGTGAACACGAATACGGGTTAC | 485 | 1..4 | 5..27 |
| Gm486 | TTTAGGGATAATGTTCTCGATGACGAA | 486 | 1..4 | 5..27 |
| Gm487 | TTTAGGTACTAACAACTCATGGAGACA | 487 | 1..4 | 5..27 |
| Gm488 | TTTAGGTGACACGCACGAATTGGTCAA | 488 | 1..4 | 5..27 |
| Gm489 | TTTGGTGAGTATGAAAGGAAGAAGCAG | 489 | 1..4 | 5..27 |
| Gm490 | TTTAGACCTCAATTAGAGGAGCTTAAG | 490 | 1..4 | 5..27 |
| Gm491 | TTTAGTACCTAATTATGACGGAACAAA | 491 | 1..4 | 5..27 |
| Gm492 | TTTGGTCTCTCGAATTTAATAACTCAT | 492 | 1..4 | 5..27 |
| Gm493 | TTTGCACTGAAGTGTAGCTCGCACGGC | 493 | 1..4 | 5..27 |
| Gm494 | TTTAGATTGAAGGTATAGATAATTGGC | 494 | 1..4 | 5..27 |
| Gm495 | TTTAGGGTTTGAACTCAGCGCGGTCCG | 495 | 1..4 | 5..27 |
| Gm496 | TTTGCTATAAACACCAACAATACTATC | 496 | 1..4 | 5..27 |
| Gm497 | TTTGGAAACTCACGAGGATCAGGATAG | 497 | 1..4 | 5..27 |
| Gm498 | TTTAGGTGTGGAAGTTGTGGCAGTAGA | 498 | 1..4 | 5..27 |
| Gm499 | TTTAGTACTGGTTATAGCTGCAGCCCT | 499 | 1..4 | 5..27 |
| Gm500 | TTTGAACGCAAATCTTATCGTAACAAC | 500 | 1..4 | 5..27 |
| Gm501 | TTTGCGTTCAAATTACAAGATGTGACC | 501 | 1..4 | 5..27 |
| Gm502 | TTTGGGATCATGTACTGTGTCTTATAA | 502 | 1..4 | 5..27 |
| Gm503 | TTTAGGTACACTGGAGGTGCAAGACAC | 503 | 1..4 | 5..27 |
| Gm504 | TTTGGGACAATAATATGACTGAGCTCC | 504 | 1..4 | 5..27 |
| Gm505 | TTTACAATGACAGATATGTAGGCCATC | 505 | 1..4 | 5..27 |
| Gm506 | TTTGGATCCGTAGCTACTCCGATCTCT | 506 | 1..4 | 5..27 |
| Gm507 | TTTGGCAGCTAAGCAATGGAGCTGCTG | 507 | 1..4 | 5..27 |
| Gm508 | TTTAGTACCACACTCCTGCATAAATAT | 508 | 1..4 | 5..27 |
| Gm509 | TTTCCTCAAACGACGTACCGGATTTCC | 509 | 1..4 | 5..27 |
| Gm510 | TTTAGAATTCTCCCGAAGTTACATCAC | 510 | 1..4 | 5..27 |
| Gm511 | TTTGAGTGTATTGGCCACAAACTTTCC | 511 | 1..4 | 5..27 |
| Gm512 | TTTAGAAGACAGACACATACATGGACA | 512 | 1..4 | 5..27 |
| Gm513 | TTTGGACTCATCGGAACGTGATCACAC | 513 | 1..4 | 5..27 |
| Gm514 | TTTCGCCGAAATCCGGTGAAACGGGCT | 514 | 1..4 | 5..27 |
| Gm515 | TTTCGGGGTGGCGGAGACAGGAGAACC | 515 | 1..4 | 5..27 |
| Gm516 | TTTCGTTGTAGTAGCAATGCTATTGAC | 516 | 1..4 | 5..27 |
| Gm517 | TTTAGAGTCACCACCTTTCAAATTACA | 517 | 1..4 | 5..27 |
| Gm518 | TTTAGTCTTACCCATCTCATTAACACT | 518 | 1..4 | 5..27 |
| Gm519 | TTTAGAAGTGATAAGACAGGAGATAAA | 519 | 1..4 | 5..27 |
| Gm520 | TTTCGACTCATACCATACATCATACTA | 520 | 1..4 | 5..27 |
| Gm521 | TTTGGAGCATTTGGGGACGTATTTATG | 521 | 1..4 | 5..27 |
| Gm522 | TTTGGGAAATGTCTATGGTGGGAGGTT | 522 | 1..4 | 5..27 |
| Gm523 | TTTAGTACGTGCATGATTGGGAAGGAA | 523 | 1..4 | 5..27 |
| Gm524 | TTTGGTGCAATAGCGAGCAGCGATGGC | 524 | 1..4 | 5..27 |
| Gm525 | TTTAGACGCATGATAGCATTCCTCATC | 525 | 1..4 | 5..27 |
| Gm526 | TTTCGGGCCATCGCTGCTCGCTATTGC | 526 | 1..4 | 5..27 |
| Gm527 | TTTCGGGCCATTCGCCAAGTACCTTTC | 527 | 1..4 | 5..27 |
| Gm528 | TTTGGGTCAATAGGTGGCGTGAATGTG | 528 | 1..4 | 5..27 |
| Gm529 | TTTGGCTCCACGACAGAGTTTGAGGAG | 529 | 1..4 | 5..27 |
| Gm530 | TTTACTGTCAATCTCTCGTTTGGCTCC | 530 | 1..4 | 5..27 |
| Gm531 | TTTGGAGAAGAAAGAGGGTAGGGTCCA | 531 | 1..4 | 5..27 |
| Gm532 | TTTCAAATCACATAGAAGAGGGCCGGC | 532 | 1..4 | 5..27 |
| Gm533 | TTTCGAACCAAATGCTTAGGAGCCAAT | 533 | 1..4 | 5..27 |
| Gm534 | TTTGGAACTCTAACATGAGGCATATAT | 534 | 1..4 | 5..27 |
| Gm535 | TTTGGCGAGAGTAGTATGTACTCAGTT | 535 | 1..4 | 5..27 |
| Gm536 | TTTAGCTAGGGACAGATTCAGGATTAT | 536 | 1..4 | 5..27 |
| Gm537 | TTTAGGAAGGTGCAACGAATGTCGTTG | 537 | 1..4 | 5..27 |
| Gm538 | TTTACGTACCTCGACGTCCGTATAATC | 538 | 1..4 | 5..27 |
| Gm539 | TTTAGCCATTGATGAAGGGTTGATGAG | 539 | 1..4 | 5..27 |
| Gm540 | TTTGGGAATACAGAAAGTTATGTAAGA | 540 | 1..4 | 5..27 |
| Gm541 | TTTGGGTGCAATTAATTAGGCTAACGA | 541 | 1..4 | 5..27 |
| Gm542 | TTTGGACCAAACCTACCCTTTGAATTC | 542 | 1..4 | 5..27 |
| Gm543 | TTTGGGATAAATTATATCACCAAAGGC | 543 | 1..4 | 5..27 |
| Gm544 | TTTAGTATATCACAAGGCCACGTCAAT | 544 | 1..4 | 5..27 |
| Gm545 | TTTGCTCTTATCATCTCCACGAAACAC | 545 | 1..4 | 5..27 |
| Gm546 | TTTAATAACAGTCGTATGGTCTAACGC | 546 | 1..4 | 5..27 |
| Gm547 | TTTGATACTACAAAGCCACGAGATGAC | 547 | 1..4 | 5..27 |
| Gm548 | TTTAGGGAGAATGTTCACATGTAGGCA | 548 | 1..4 | 5..27 |
| Gm549 | TTTCGTGACCTTTGGGTGGCAGTGAAC | 549 | 1..4 | 5..27 |
| Gm550 | TTTGGACTATGTGTTTCAGTTAGAAAC | 550 | 1..4 | 5..27 |
| Gm551 | TTTGGAGTGGTGTCAACGTGAATACGA | 551 | 1..4 | 5..27 |
| Gm552 | TTTAGCATTTGTTATGAACATACGGAC | 552 | 1..4 | 5..27 |
| Gm553 | TTTGGTATGAGAGTTTATGAATATTCG | 553 | 1..4 | 5..27 |
| Gm554 | TTTGGAGCTAGTAAGAGATAAATTTCC | 554 | 1..4 | 5..27 |
| Gm555 | TTTGGAACTTACCTGGCATAAATACTC | 555 | 1..4 | 5..27 |
| Gm556 | TTTAGCTGTAATCAATTGAAATACCAG | 556 | 1..4 | 5..27 |
| Gm557 | TTTGGTTTCATATCTCACTCCCAATTT | 557 | 1..4 | 5..27 |
| Gm558 | TTTGGAAATTTATCTCTTACTAGCTCC | 558 | 1..4 | 5..27 |
| Gm559 | TTTCGATGCAAGAAAGGAAGTAGTGTG | 559 | 1..4 | 5..27 |
| Gm560 | TTTGGGGACACATGTCTATGTGAGTTG | 560 | 1..4 | 5..27 |
| Gm561 | TTTCGTTCATGTGGCACGTCGGTCATG | 561 | 1..4 | 5..27 |
| Gm562 | TTTAAGTGGATTCACGGTGGAGGGAAC | 562 | 1..4 | 5..27 |
| Gm563 | TTTGGAACAACTTAATAGGGGTATCCT | 563 | 1..4 | 5..27 |
| Gm564 | TTTAGAATTTGTGCAAGTAATCCCTGC | 564 | 1..4 | 5..27 |
| Gm565 | TTTCGACCAGATGCAGGGATTACTTGC | 565 | 1..4 | 5..27 |
| Gm566 | TTTAGTAGCTGACCAAGTTACCCAACA | 566 | 1..4 | 5..27 |
| Gm567 | TTTAATAATATGTGTCAATCTCATACC | 567 | 1..4 | 5..27 |
| Gm568 | TTTAGTTCTCTTCCTAACCTCACGCGA | 568 | 1..4 | 5..27 |
| Gm569 | TTTGGTCTGATACCACTACTCAGCTGC | 569 | 1..4 | 5..27 |
| Gm570 | TTTAGTAGGAGGGACTTGACCTCGAAC | 570 | 1..4 | 5..27 |
| Gm571 | TTTGGACCTACAACATGCATTCTACTC | 571 | 1..4 | 5..27 |
| Gm572 | TTTAGAAGAGGTTGTGGTTGGGTGGAC | 572 | 1..4 | 5..27 |
| Gm573 | TTTAGAAAGGAAGTTGTCGCTGAAACC | 573 | 1..4 | 5..27 |
| Gm574 | TTTGGACATTTGGTCTAGTGGCACGGG | 574 | 1..4 | 5..27 |
| Gm575 | TTTGGCCATAAACATCGATCTGTGTCG | 575 | 1..4 | 5..27 |
| Gm576 | TTTAGGCGTGAGTATAACCCGGTTGGT | 576 | 1..4 | 5..27 |
| Gm577 | TTTGGTGGGGTTGGGACGTGCGGTGGT | 577 | 1..4 | 5..27 |
| Gm578 | TTTACGTTTACATCTGGAGGTGGAAAC | 578 | 1..4 | 5..27 |
| Gm579 | TTTAGTAGCATCTCACCTTGTGGAGAT | 579 | 1..4 | 5..27 |
| Gm580 | TTTGGTCTAGTGGCACGGGTCTTGCTT | 580 | 1..4 | 5..27 |
| Gm581 | TTTGGATGCCCATCTTCCTTGTGAGGT | 581 | 1..4 | 5..27 |
| Gm582 | TTTGGCGACATTATAATTAGAAAGACA | 582 | 1..4 | 5..27 |
| Gm583 | TTTGGGAATAGAACACTTTAGTAAAGA | 583 | 1..4 | 5..27 |
| Gm584 | TTTGGGGTAGGAAAGAAAGAACAAGGG | 584 | 1..4 | 5..27 |
| Gm585 | TTTAGGTCATTAATTGCAGAGCGAAGA | 585 | 1..4 | 5..27 |
| Gm586 | TTTAGTACGAGTTAAAGATGTATCGAT | 586 | 1..4 | 5..27 |
| Gm587 | TTTCGTCCATCCCACCATGCTCAATAA | 587 | 1..4 | 5..27 |
| Gm588 | TTTCGATAAAGTGAAAGAACATCAACC | 588 | 1..4 | 5..27 |
| Gm589 | TTTGGCACAGCTTGGGATTCGACGTCT | 589 | 1..4 | 5..27 |
| Gm590 | TTTAGTGTCTCAAAGCAGAATATGCGT | 590 | 1..4 | 5..27 |
| Gm591 | TTTGGGATTAACATCTTTCGATAAAGT | 591 | 1..4 | 5..27 |
| Gm592 | TTTGCGCACACGTACCAGCAAAGAACC | 592 | 1..4 | 5..27 |
| Gm593 | TTTGGGTCGGAGAGAATAGTCACAAGC | 593 | 1 .. | 5..27 |
| Gm594 | TTTACACAAATAACACAAAGCAATCAC | 594 | 1..4 | 5..27 |
| Gm595 | TTTAGTGTCATTGCTGACACGATTCAT | 595 | 1..4 | 5..27 |
| Gm596 | TTTGCTGGTACGTGTGCGCAAATTTAC | 596 | 1..4 | 5..27 |
| Gm597 | TTTGGTGGAAACTACAAGCTTAGTCAC | 597 | 1..4 | 5..27 |
| Gm598 | TTTGGCAGCAAGTATGATATGGGCGTG | 598 | 1..4 | 5..27 |
| Gm599 | TTTCGTTCCATATGGAAAGGTGTTGGC | 599 | 1..4 | 5..27 |
| Gm600 | TTTACACTGATAGTGACAGGACTTAAC | 600 | 1..4 | 5..27 |
| Gm601 | TTTAGTCAGCCACAGTCAACTAATGCG | 601 | 1..4 | 5..27 |
| Gm602 | TTTACTGCCTCCGTGCCACGTGTGATC | 602 | 1..4 | 5..27 |
| Gm603 | TTTAGCTAGATCAGTACGCAATTTAAA | 603 | 1..4 | 5..27 |
| Gm604 | TTTAGTAACTCATGGAGGATTGAATTC | 604 | 1..4 | 5..27 |
| Gm605 | TTTAGTCTTCATCATATGTCCGCATTC | 605 | 1..4 | 5..27 |
| Gm606 | TTTGGTGCATCGCAAGAAATGTATATA | 606 | 1..4 | 5..27 |
| Gm607 | TTTAGTTATATACGCATCAGCATCTAT | 607 | 1..4 | 5..27 |
| Gm608 | TTTCGAACAATGTTGCCAGAGCGTGAT | 608 | 1..4 | 5..27 |
| Gm609 | TTTCGATTATGCATGCACTGACAAGGC | 609 | 1..4 | 5..27 |
| Gm610 | TTTGGCAAATTGGCATAGTTGGGGTAG | 610 | 1..4 | 5..27 |
| Gm611 | TTTCGAAGAAGATAAAGTGATGAACTC | 611 | 1..4 | 5..27 |
| Gm612 | TTTCGCAACTGCCATCGTTGGCCATTC | 612 | 1..4 | 5..27 |
| Gm613 | TTTGACACGACCAAACACAATCCAAAC | 613 | 1..4 | 5..27 |
| Gm614 | TTTGTCCTAACCATGAAATGGGCTCCC | 614 | 1..4 | 5..27 |
| Gm615 | TTTAGACGCTTGATTCAGAAACAGGAT | 615 | 1..4 | 5..27 |
| Gm616 | TTTGGTGCTAGAATAGAAAGCATTAGA | 616 | 1..4 | 5..27 |
| Gm617 | TTTGTTCTCAGTGCTGCCAACCTAAGC | 617 | 1..4 | 5..27 |
| Gm618 | TTTGGACCAATCAGAGTGGAACTTATC | 618 | 1..4 | 5..27 |
| Gm619 | TTTCGTCAAATCCGATTCAAACTAAAC | 619 | 1..4 | 5..27 |
| Gm620 | TTTGGACGGTTAACGGCAATATAATCC | 620 | 1..4 | 5..27 |
| Gm621 | TTTGGAGCTACGTTGGTCAGCTCTTTG | 621 | 1..4 | 5..27 |
| Gm622 | TTTAGGAATTGCTATGGGCACGCACGT | 622 | 1..4 | 5..27 |
| Gm623 | TTTGGCAGTGTTGGTCGTTTGAGACGT | 623 | 1..4 | 5..27 |
| Gm624 | TTTAGTACAAGTATTGTGTTGTTGGCC | 624 | 1..4 | 5..27 |
| Gm625 | TTTGAGTATAAATCATGGGAATGTCCC | 625 | 1..4 | 5..27 |
| Gm626 | TTTGCCAGTAGGATCCTTACATCTTGC | 626 | 1..4 | 5..27 |
| Gm627 | TTTGGACTGACCATATTATATAAGGAG | 627 | 1..4 | 5..27 |
| Gm628 | TTTAGCTAGTATAAGCAACTATCAAGG | 628 | 1..4 | 5..27 |
| Gm629 | TTTAGGCATATTATGAATCACCGATGA | 629 | 1..4 | 5..27 |
| Gm630 | TTTGGTCTCTGTTACCTAGGTCCTAAG | 630 | 1..4 | 5..27 |
| Gm631 | TTTCGTGCTAGTTCCTTGCTTCACATC | 631 | 1..4 | 5..27 |
| Gm632 | TTTAGTGGCATGTTGATAGTTGCAGAG | 632 | 1..4 | 5..27 |
| Gm633 | TTTAGAAATACTCAATCTCATATAGCC | 633 | 1..4 | 5..27 |
| Gm634 | TTTAGACAGATATTAACCTAAACATAC | 634 | 1..4 | 5..27 |
| Gm635 | TTTAGCACCACCATCCAAGATTAAGTC | 635 | 1..4 | 5..27 |
| Gm636 | TTTAGCACCATGTATTGAGAGAGAGTC | 636 | 1..4 | 5..27 |
| Gm637 | TTTGGAAGAAACACTTATGTCAGTCAC | 637 | 1..4 | 5..27 |
| Gm638 | TTTAGCGAGTTGGAACGACCCATGTCC | 638 | 1..4 | 5..27 |
| Gm639 | TTTGGTGCTTAAGACAACTGGGCTTGC | 639 | 1..4 | 5..27 |
| Gm640 | TTTGGAAGGTCTAAACTCCTCCAAGAC | 640 | 1..4 | 5..27 |
| Gm641 | TTTGGACTATGTGAGCAAGGAATGATC | 641 | 1..4 | 5..27 |
| Gm642 | TTTGGCACGATATGGCGATATGAACTA | 642 | 1..4 | 5..27 |
| Gm643 | TTTAGCCTGTGGTTTAGAGGAGAACCA | 643 | 1..4 | 5..27 |
| Gm644 | TTTCCGGGTAAGATCGAGGGTTCAAAC | 644 | 1..4 | 5..27 |
| Gm645 | TTTGGCTTTATTCAGAACCAGAAGCCT | 645 | 1..4 | 5..27 |
| Gm646 | TTTAGGGTAAGCACGAACAGGAAAGTT | 646 | 1..4 | 5..27 |
| Gm647 | TTTAAAGAGAGGTAGAATCGTAGAAGC | 647 | 1..4 | 5..27 |
| Gm648 | TTTACAAACATACCATAAATAGTTCCC | 648 | 1..4 | 5..27 |
| Gm649 | TTTAGAATAACATCACACTGCAAGAAG | 649 | 1..4 | 5..27 |
| Gm650 | TTTAGAATCCACCAACACCAAGACTGT | 650 | 1..4 | 5..27 |
| Gm651 | TTTGGACCTTTCTGGGTACGTATTGGG | 651 | 1..4 | 5..27 |
| Gm652 | TTTGGATATAAAGTCCTCTCTTTCAAT | 652 | 1..4 | 5..27 |
| Gm653 | TTTCGCATGGAGTGAAAGGCTTCTTGC | 653 | 1..4 | 5..27 |
| Gm654 | TTTAGCTTTAAAGAGAGGTAGAATCGT | 654 | 1..4 | 5..27 |
| Gm655 | TTTAGGGAGATGGATACATTCAGAGAG | 655 | 1..4 | 5..27 |
| Gm656 | TTTAGTGTAACGTGGATTACTGAACTT | 656 | 1..4 | 5..27 |
| Gm657 | TTTGGTTAGTCCAGTACTCCTGTTGAC | 657 | 1..4 | 5..27 |
| Gm658 | TTTAGAAATATTGAAGTCATTGCTTGC | 658 | 1..4 | 5..27 |
| Gm659 | TTTAGACCTTCCAAAGGGATTTGTAAG | 659 | 1..4 | 5..27 |
| Gm660 | TTTGGAGGGTTGATGCTAAACATGGTG | 660 | 1..4 | 5..27 |
| Gm661 | TTTAGATGATGAGATGACAGGTTGGGT | 661 | 1..4 | 5..27 |
| Gm662 | TTTGGATGTTTAGCTCAAACGAACTTT | 662 | 1..4 | 5..27 |
| Gm663 | TTTCGCTCAACACCACGACATAGTTAT | 663 | 1..4 | 5..27 |
| Gm664 | TTTGGGAGTGCTTTGACTCGAAGACAT | 664 | 1..4 | 5..27 |
| Gm665 | TTTAGGTACATATTCGGAACTTGGAAA | 665 | 1..4 | 5..27 |
| Gm666 | TTTAGTAGATGGGGTGGAGTAAATTGT | 666 | 1..4 | 5..27 |
| Gm667 | TTTGGTTTGTAGACCGAAGTCACCGAA | 667 | 1..4 | 5..27 |
| Gm668 | TTTGTCAACAGGAGTACTGGACTAACC | 668 | 1..4 | 5..27 |
| Gm669 | TTTAGTACGATTGTACATGTAATTAAC | 669 | 1..4 | 5..27 |
| Gm670 | TTTAGATTGGCCCAGCGAAGGAGGCAT | 670 | 1..4 | 5..27 |
| Gm671 | TTTGGCGAATGCTATGAGTGTGACTTC | 671 | 1..4 | 5..27 |
| Gm672 | TTTAGGTAAGACCGGTGCACATGAACG | 672 | 1..4 | 5..27 |
| Gm673 | TTTGAGATAATTAGGATCAAGATCTCC | 673 | 1..4 | 5..27 |
| Gm674 | TTTACGTCTATTTCAAGATTAATCAAC | 674 | 1..4 | 5..27 |
| Gm675 | TTTGGCGTATATGCTGTTTGTGTTGGT | 675 | 1..4 | 5..27 |
| Gm676 | TTTGGCTAACGGTAAAGACAAGAATGT | 676 | 1..4 | 5..27 |
| Gm677 | TTTAGCTAACTCCATCATAGTGCTGTC | 677 | 1..4 | 5..27 |
| Gm678 | TTTGGATGTTTGCGCACCATGCATATG | 678 | 1..4 | 5..27 |
| Gm679 | TTTAGATTGTGTTATCAAGGGTCAACT | 679 | 1..4 | 5..27 |
| Gm680 | TTTGGCATCAGACATAAGCAACAAGCC | 680 | 1..4 | 5..27 |
| Gm681 | TTTAGATATGCTGCTCACGTGGGCAGC | 681 | 1..4 | 5..27 |
| Gm682 | TTTAGCATGGTCTACGCGCGGAACTTC | 682 | 1..4 | 5..27 |
| Gm683 | TTTAGTTTAATGATATAGAACCACTAC | 683 | 1..4 | 5..27 |
| Gm684 | TTTGGAGGCCAACATAGGTAGCTACCT | 684 | 1..4 | 5..27 |
| Gm685 | TTTAGGACCTAGGACCACGGTACTTAA | 685 | 1..4 | 5..27 |
| Gm686 | TTTGGCACACGTGTGCTGAATGTGACG | 686 | 1..4 | 5..27 |
| Gm687 | TTTGGTCTCAGGACAAGGTTGCTTTGG | 687 | 1..4 | 5..27 |
| Gm688 | TTTAGAATATGGTAAATGGTTCAATTC | 688 | 1..4 | 5..27 |
| Gm689 | TTTGGGTCTGCCAATGTCATCCTGATG | 689 | 1..4 | 5..27 |
| Gm690 | TTTGGTCGATATAGACCGCATTATATC | 690 | 1..4 | 5..27 |
| Gm691 | TTTGGTGCATCTGAATGATGAGCAGAG | 691 | 1..4 | 5..27 |
| Gm692 | TTTGGCAGGTTTACTGTGTTCTCACTG | 692 | 1..4 | 5..27 |
| Gm693 | TTTAGGTGAAGTTTATAAGCCTTGAAT | 693 | 1..4 | 5..27 |
| Gm694 | TTTAGTGGGACGGGTCAAACCCATTCC | 694 | 1..4 | 5..27 |
| Gm695 | TTTAGTCCGAATGGTGAACGGGACTCT | 695 | 1..4 | 5..27 |
| Gm696 | TTTAGGGGATTGCAGGGTGTCTACCGG | 696 | 1..4 | 5..27 |
| Gm697 | TTTAGTTTCACCAGCCTCTCGTTATTC | 697 | 1..4 | 5..27 |
| Gm698 | TTTAGAAACAACGGTAAAGCATATTAA | 698 | 1..4 | 5..27 |
| Gm699 | TTTAACAGAATATTTCAGCCGGTAGAC | 699 | 1..4 | 5..27 |
| Gm700 | TTTAGTACATCTACAACAAAGCAAAGG | 700 | 1..4 | 5..27 |
| Gm701 | TTTGGTCGGAGCCTGTTACCGGAACAG | 701 | 1..4 | 5..27 |
| Gm702 | TTTAGTCATAAGTAACAATGGTAGCAT | 702 | 1..4 | 5..27 |
| Gm703 | TTTGATCATACAAGCGTTTATAAACCC | 703 | 1..4 | 5..27 |
| Gm704 | TTTGGTTACCATGGAAGCCATGTATCA | 704 | 1..4 | 5..27 |
| Gm705 | TTTCGCTCGCTATATATATGTGTCACA | 705 | 1..4 | 5..27 |
| Gm706 | TTTGCAAACATCACATGGCCGGATAAG | 706 | 1..4 | 5..27 |
| Gm707 | TTTACATGCTGTCATCATCTGTTCACT | 707 | 1..4 | 5..27 |
| Gm708 | TTTACCTTGATTTGGATTAACCCGCCA | 708 | 1..4 | 5..27 |
| Gm709 | TTTACTTCCTGAGGAAATTTGGTTTCA | 709 | 1..4 | 5..27 |

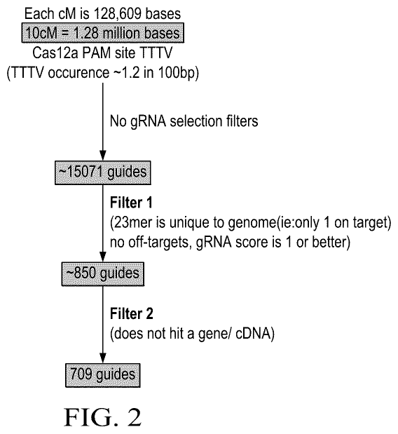

The −10 cM genomic region surrounding the soybean Gm_CSM63714 event on Chromosome 13 comprises 1.29 million bases. The TTTV Cas12a PAM sequence is expected to occur randomly once every about 100 or so bases. Thus, there are 15071 guide RNA target sites in the 10 cM region. This necessitates the need for robust filters to select for optimal guide RNA target sites. The filters described in FIG. 2 narrowed down the testable guides to 709.

### Example 2. Nuclease Cutting Rates of Soybean Genomic Target Sites

The Cas12a gRNA target sites identified herein are further evaluated for nuclease cutting rates. Cas12a gRNAs that can hybridize to each target site are designed and cloned into a plant expression vector along with Cas12a nuclease and delivered to the soy plant. Delivery methods of DNA-based expression vectors include but are not limited to (1) polyethylene-glycol (PEG) mediated protoplast transformation, (2) Agrobacterium-mediated transformation, (3) particle bombardment, and (4) carbon nanoparticle delivery and (5) viral delivery. The editing components can also be delivered as ribonucleo-protein (RNP) complexes that are assembled in vitro, prior to transformation.

Agrobacterium T-DNA vectors are designed to evaluate gRNA and Cas12a nuclease mediated cleavage at each of the selected target sites. Each T-DNA vector comprises 3 cassettes. The first cassette comprises a plant codon-optimized LbCas12a flanked by nuclear localization signal sequences operably linked to promoter and a 3′UTR sequence. The second cassette comprises a unique gRNA cassette targeting one or more of the 709 targeting sites described in Table 1 (SEQ ID NO: 1-709). The cassette comprises a Polymerase III promoter that is functional in soy cells operably linked to a Cas12a gRNA unit. Each gRNA unit comprises a single G leader followed by a crRNA scaffold (also called a direct repeat) sequence (AATTTCTACTAAGTGTAGAT (SEQ ID NO:710) or TAATTTCTACTAAGTGTAGAT (SEQ ID NO:711)) compatible with LbCas12a, at least one 23 bp unique gRNA hybridization sequence (also called a spacer sequence) that when transcribed results in a sequence that is complementary to and hybridizes with a unique genomic target site listed in Table 1, a 20-21 bp downstream mature crRNA scaffold and a 7 bp poly T sequence. In addition, gRNA arrays can be designed for multiplexed editing of multiple target sites. For example, the gRNA unit could comprise two or more unique spacer sequences separated by crRNA scaffolds. The third cassette is a selectable marker cassette, for example, an EPSPS expression cassette encoding a 5-enolpyruvylshikimate-3-phosphate synthase selectable marker for selecting transformants in the presence of glyphosate.

Soy embryo explants are transformed with the vectors described above by Agrobacterium-mediated transformation. Transformed plants are selected on glyphosate. Leaf samples from regenerated plantlets are harvested and genomic DNA is extracted. PCR-based assays are performed using a pair of PCR primers flanking the intended target region. PCR products are sequenced and analyzed to identify edits in the target site. Editing rates for each target site are calculated by the number of plants containing an edit/number of plants returning data and multiplying by 100. Editing rates are used to identify gRNAs and corresponding target sites with top editing rates.

Fragment Length Analysis (FLA) is another assay that can be used to identify edits. FLA is a PCR-based molecular assay that can be used to identify indel (insertion or deletion) mutations introduced at the target site by NHEJ-mediated (Non-Homologous End Joining) DNA repair following dsDNA cleavage by the LbCas12a-guide complex. Genomic DNA is subjected to a PCR reaction with primers flanking each target site to generate amplicons. The amplicon fragment lengths are subsequently analyzed using capillary electrophoresis, and compared to a wild-type amplicon to identify mutants that had larger or smaller amplicons due to the presence of indels. Editing rates for each target site are calculated by the number of plants containing an edit/number of plants returning data multiplied by 100. Editing rates are used to identify gRNAs and corresponding target sites with top editing rates.

The target sites are also evaluated for site directed integration of the T-DNA. In a subset of cells, the T-DNA can serve as a donor DNA and integrate into the chromosomal target site subsequent to LbCas12a- and gRNA-mediated cleavage. To identify plants with site directed integration of the T-DNA at a soy target site, flanking PCR assays similar to those described in PCT Patent Application No. WO 2019/084148 using PCR primers flanking the intended target site are performed. For flank PCR, primers are designed to identify SDI events with either forward or reverse orientation T-DNA insertions. Thus, four assays are designed for each construct/target site combination. Genomic DNA from positive flanking PCR results indicating putative SDI events are sequenced to confirm T-DNA insertion.

Having described the present disclosure in detail, it will be apparent that modifications, variations, and equivalent embodiments are possible without departing from the spirit and scope of the present disclosure as described herein and in the appended claims. Furthermore, it should be appreciated that all examples in the present disclosure are provided as non-limiting examples.

1. A recombinant DNA molecule comprising a DNA sequence having at least 85% sequence identity, at least 90% sequence identity, or at least 95% sequence identity to a nucleic acid molecule selected from the group consisting of nucleotides 5-27 of SEQ ID NOs:1-709.

2. The recombinant DNA molecule of claim 1, comprising a nucleic acid molecule selected from the group consisting of nucleotides 5-27 of SEQ ID NOs:1-709.

3. The recombinant DNA molecule of claim 1, wherein said DNA sequence is operably linked to a heterologous promoter sequence.

4. The recombinant DNA molecule of claim 3, further comprising SEQ ID NO:710 or SEQ ID NO:711.

5. A recombinant RNA molecule comprising an RNA sequence that is at least 85% complementary, at least 90% complementary, or at least 95% complementary to a nucleic acid molecule selected from the group consisting of nucleotides 5-27 of SEQ ID NOs:1-709.

6. The recombinant RNA molecule of claim 5, wherein the RNA sequence is 100% complementary to a nucleic acid molecule selected from the group consisting of nucleotides 5-27 of SEQ ID NOs:1-709.

7. A soy plant, plant seed, plant part, plant cell or progeny plant comprising a recombinant nucleic acid molecule, said recombinant nucleic acid molecule comprising a target soy genomic nucleic acid sequence having at least 85% sequence identity, at least 90% sequence identity, or at least 95% sequence identity to a nucleic acid molecule selected from the group consisting of SEQ ID NOs:1-709; and a DNA sequence of interest, wherein the DNA sequence of interest is inserted into said target soy genomic nucleic acid sequence.

8. The soy plant, seed, plant part, plant cell or progeny plant of claim 7, comprising a recombinant nucleic acid molecule, said recombinant nucleic acid molecule comprising a target soy genomic nucleic acid sequence having a sequence selected from the group consisting of SEQ ID NOs:1-709.

9. The soy plant, seed, plant part, plant cell or progeny plant of claim 7, wherein the DNA sequence of interest comprises a gene of agronomic interest.

10. The soy plant, seed, plant part, plant cell or progeny plant of claim 9, wherein the gene of agronomic interest confers herbicide tolerance in plants.

11. The soy plant, seed, plant part, plant cell or progeny plant of claim 7, wherein the target soy genomic nucleic acid sequence is at least 1 kb from a soy event Gm_CSM63714 insertion site.

12. The soy plant, seed, plant part, plant cell or progeny plant of claim 7, wherein the target soy genomic nucleic acid sequence is at least 1 cM from a soy event Gm_CSM63714 insertion site.

13. The soy plant, seed, plant part, plant cell or progeny plant of claim 11, wherein the target soy genomic nucleic acid sequence maps to within 5 cM of the soy event Gm_CSM63714 insertion site.

14. A method of generating a recombinant soy plant cell comprising the following steps:
a. obtaining a soy plant, seed, or cell, wherein said plant, seed, or cell comprises a target soy genomic nucleic acid molecule having at least 85% sequence identity, at least 90% sequence identity, or at least 95% sequence identity to a nucleic acid molecule selected from the group consisting of SEQ ID NOs:1-709;
b. introducing into the soy plant, seed, or cell a site-specific nuclease that can specifically bind to and cleave the target soy genomic nucleic acid molecule;
c. introducing a DNA sequence of interest into the soy plant, seed, or cell;
d. inserting the DNA sequence of interest into the target soy genomic nucleic acid molecule; and
e. selecting recombinant soy plants, seeds or cells comprising the DNA sequence of interest inserted in the target soy genomic nucleic acid molecule.

15. The method of claim 14, wherein the site-specific nuclease is selected from the group consisting of an RNA-guided nuclease, a zinc finger nuclease and a TALEN.

16. The method of claim 15, where the RNA-guided nuclease is Cas12a.

17. The method of claim 16 further comprising introducing into the soy plant, seed, or cell a guide polynucleotide comprising a nucleic acid sequence that is substantially complementary to the target soy genomic nucleic acid, wherein the guide polynucleotide and the RNA-guided nuclease form a complex that can bind to and cleave the soy genomic nucleic acid molecule.

18. The method of claim 17, wherein the guide polynucleotide comprises a nucleotide sequence having at least at least 85% sequence identity, at least 90% sequence identity, or at least 95% sequence identity to a nucleic acid molecule selected from the group consisting of nucleotides 5-27 of SEQ ID NOs:1-709.

19. The method of claim 16, wherein the guide polynucleotide further comprises SEQ ID NO:710 or SEQ ID NO:711.

20. The method of claim 14, wherein the target soy genomic nucleic acid molecule is at least 1 kb from a soy event Gm_CSM63714 insertion site.

21. The method of claim 14, wherein the target soy genomic nucleic acid molecule is at least 1 cM from a soy event Gm_CSM63714 insertion site.

22. The method of claim 20, wherein the target soy genomic nucleic acid sequence maps to within 5 cM of the soy event Gm_CSM63714 insertion site.
